(19) United States
(12) Patent Application Publication
(10) Pub. No.: US 2024/0366661 A1
(43) Pub. Date: Nov. 7, 2024

(54) COMPOSITIONS FOR AND METHODS OF TREATING A SUBJECT HAVING INFLAMMATION

(71) Applicant: The George Washington University, Washington, DC (US)

(72) Inventors: Brett SHOOK, Washington, DC (US); Alejandro VILLAGRA, Washington, DC (US); Satish Kumar R. NOONEPALLE, Washington, DC (US); Maria HERNANDEZ, Washington, DC (US)

(21) Appl. No.: 18/774,580

(22) Filed: Jul. 16, 2024

Related U.S. Application Data
(63) Continuation of application No. PCT/US2023/011454, filed on Jan. 24, 2023.
(60) Provisional application No. 63/267,258, filed on Jan. 28, 2022.

(51) Int. Cl. A61K 35/15; A61K 45/06; C12N 5/0786

(52) U.S. Cl. CPC A61K 35/15; A61K 45/06; C12N 5/0645

(57) ABSTRACT

Disclosed herein are compositions for use in and methods of producing HDAC11-activated macrophages. Also disclosed herein are methods of treating inflammation and providing adaptive cell therapy comprising HDAC11-activated M2 macrophages.

### CROSS-REFERENCE TO RELATED APPLICATIONS

This application is a continuation of PCT Application No. PCT/US2023/011454, filed Jan. 24, 2023, which claims priority to U.S. Provisional Application No. 63/267,258, filed Jan. 28, 2022, each of which is incorporated herein in its entirety.

### BACKGROUND

Macrophages are critical immune cells involved in both adaptive and innate immune responses. Tissue-resident macrophages are primarily derived from two main sources: (i) embryonic yolk sac-derived myeloid progenitor cells, and (ii) tissue infiltrated circulating monocytes. Macrophages can be classified into pro-inflammatory M1 and anti-inflammatory M2 macrophages based on cytokine profiles and inflammatory status. Tissue-resident macrophages play an essential role in regulating local inflammatory responses to insults resulting in an injury and subsequent healing process. This requires a series of coordinated events, which begins with inflammation mediated by M1 macrophages and ends with healing response, including the processes of regeneration, angiogenesis, and extracellular matrix remodeling mediated by M2 macrophages, indicating that macrophages are intricately involved in healing processes. Therefore, controlling the inflammatory status of the macrophages can have a profound influence on the wound healing process, and defective wound healing is often attributed to the pro-inflammatory nature of M1 macrophages.

Thus, there remains an urgent need to diminish, decrease, and/or control the inflammation associated with many diseases, disorders, and conditions. Consequently, the present disclosure provides compositions for and methods of diminishing and/or controlling inflammation with HDAC11-activated M2 macrophages.

### BRIEF DESCRIPTION OF THE FIGURES

FIG. 1A-FIG. 1C shows that HDAC11 regulated IL10 gene expression. FIG. 1A shows the luciferase reporter assay with distal IL10 gene promoter in RAW264.7 macrophages transfected with GFP or HDAC11 overexpression plasmids. FIG. 1B shows chromatin immunoprecipitation assay confirming HDAC11 binding to the proximal promoter region (top) and distal promoter region (bottom) of IL10 gene in RAW264.7 macrophages infected with adenovirus expressing GFP or HDAC11. FIG. 1C shows elevenostat dose dependent expression of IL-10 (top) and TGFβ (bottom) cytokines by PEMs measured by ELISA.

FIG. 2A-FIG. 2C show that HDAC11 inhibition augmented M2 phenotype. FIG. 2A shows the QRT-PCR analysis of Fizz1, FIG. 2B shows the QRT-PCR analysis of Arg1, and FIG. 2C shows the QRT-PCR analysis of Mrc1 expression in murine bone marrow derived macrophages (BMDMs) after polarization to M2 phenotype. Macrophages were pre-treated with the HDAC11 inhibitor Garcinol (5 μM), SIS17 (5 μM) and FT895 (5 μM) prior to M2 polarization.

### BRIEF SUMMARY

Disclosed herein are HDAC11-activated macrophages. Disclosed herein are HDAC11-activated M2 macrophages. Disclosed herein are HDAC11-activated macrophages made by a method disclosed herein. Disclosed herein are HDAC11-activated M2 macrophages made by a method disclosed herein. Disclosed herein are HDAC11-activated macrophages made by a method comprising contacting a population of naïve macrophages with one or more HDAC11 inhibitors. Disclosed herein are HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein are HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein are HDAC11-activated macrophages made by a method comprising contacting a population of polarized macrophages with one or more HDAC11 inhibitors. Disclosed herein are HDAC11-activated macrophages made by a method comprising contacting a population of polarized macrophages with one or more HDAC11 inhibitors, wherein the macrophages are polarized towards an anti-inflammatory phenotype. Disclosed herein are HDAC11-activated macrophages made by a method comprising contacting a population of polarized macrophages with one or more HDAC11 inhibitors, wherein the macrophages are polarized towards a M2 phenotype.

Disclosed herein are cryopreserved HDAC11-activated macrophages. Disclosed herein are cryopreserved HDAC11-activated M2 macrophages. Disclosed herein are cryopreserved HDAC11-activated macrophages made by a method disclosed herein. Disclosed herein are cryopreserved HDAC11-activated M2 macrophages made by a method disclosed herein. Disclosed herein are cryopreserved HDAC11-activated macrophages made by a method comprising contacting a population of naïve macrophages with one or more HDAC11 inhibitors. Disclosed herein are cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein are cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein are cryopreserved HDAC11-activated macrophages made by a method comprising contacting a population of polarized macrophages with one or more HDAC11 inhibitors. Disclosed herein are cryopreserved HDAC11-activated macrophages made by a method comprising contacting a population of polarized macrophages with one or more HDAC11 inhibitors, wherein the macrophages are polarized towards an anti-inflammatory phenotype. Disclosed herein are cryopreserved HDAC11-activated macrophages made by a method comprising contacting a population of polarized macrophages with one or more HDAC11 inhibitors, wherein the macrophages are polarized towards a M2 phenotype.

Disclosed herein is a pharmaceutical formulation comprising one or more disclosed HDAC11-activated M2 macrophages, and one or more pharmaceutically acceptable carrier. Disclosed herein is a pharmaceutical formulation comprising one or more disclosed composition comprising HDAC11-activated macrophages, and one or more pharmaceutically acceptable carrier. Disclosed herein is a pharmaceutical formulation comprising one or more disclosed cryopreserved HDAC11-activated M2 macrophages, and one or more pharmaceutically acceptable carrier. Disclosed herein is a pharmaceutical formulation comprising one or more disclosed composition comprising cryopreserved HDAC11-activated macrophages, and one or more pharmaceutically acceptable carrier.

Disclosed herein is method of producing HDAC11-activated macrophages, the method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein is a method of producing HDAC11-activated macrophages, the method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein is a method of producing HDAC11-activated macrophages, the method comprising contacting a population of polarized macrophages with one or more HDAC11 inhibitors. Disclosed herein is a method of producing HDAC11-activated macrophages, the method comprising contacting a population of polarized macrophages with one or more HDAC11 inhibitors, wherein the macrophages are polarized towards an anti-inflammatory phenotype. Disclosed herein is a method of producing HDAC11-activated macrophages, the method comprising contacting a population of polarized macrophages with one or more HDAC11 inhibitors, wherein the macrophages are polarized towards a M2 phenotype.

Disclosed herein is a method of treating a subject, the method comprising administering to a subject in need thereof a therapeutically effective amount of HDAC11-activated M2 macrophages or a composition comprising HDAC11-activated M2 macrophages. Disclosed herein is a method of treating a subject, the method comprising administering to a subject in need thereof a therapeutically effective amount of HDAC11-activated M2 macrophages or a composition comprising HDAC11-activated M2 macrophages, wherein following the administering step, inflammation in one or more of the subject's body systems is decreased and/or diminished. Disclosed herein is a method of treating a subject, the method comprising administering to a subject in need thereof a therapeutically effective amount of HDAC11-activated M2 macrophages or a composition comprising HDAC11-activated M2 macrophages, thereby decreasing and/or diminishing inflammation in one or more of the subject's body systems. Disclosed herein is a method of treating a subject, the method comprising administering to a subject in need thereof a therapeutically effective amount of HDAC11-activated M2 macrophages or a composition comprising HDAC11-activated M2 macrophages, and decreasing and/or diminishing inflammation in one or more of the subject's body systems.

Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages. Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, wherein following the administering step, inflammation in one or more of the subject's body systems is decreased and/or diminished.

Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, thereby decreasing and/or diminishing inflammation in one or more of the subject's body systems. Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, and decreasing and/or diminishing inflammation in one or more of the subject's body systems.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5 and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration of disclosed HDAC11-activated macrophages and/or a disclosed pharmaceutical formulation and/or a disclosed compositive. In an aspect, a subject can be a human subject. In an aspect, a subject can have a disease or disorder characterized by excessive and/or uncontrollable inflammation in one or more body systems. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered.

As used herein, the term “diagnosed” means having been subjected to an examination by a person of skill, for example, a physician, and found to have a condition (such as an inflammatory disease and/or disorder) that can be diagnosed or treated by one or more of the disclosed compositions (e.g., HDAC11-activated M2 macrophage, a composition thereof, or a pharmaceutical formulation thereof) or by one or more of the disclosed methods. For example, “diagnosed with a disease or disorder characterized by excessive and/or uncontrollable inflammation” means having been subjected to an examination by a person of skill, for example, a physician, and found to have a condition (e.g., inflammatory disorder and/or disorder) that can be treated by one or more of the disclosed compositions or by one or more of the disclosed methods. For example, “suspected of having a disease or disorder characterized by excessive and/or uncontrollable inflammation” can mean having been subjected to an examination by a person of skill, for example, a physician, and found to have a condition (e.g., inflammation) that can likely be treated by one or more of the disclosed compositions or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

A “patient” refers can refer to a subject afflicted with a disease or disorder such as a disease or disorder characterized by excessive and/or uncontrollable inflammation. In an aspect, a patient can refer to a subject that has been diagnosed with or is suspected of having a disease or disorder characterized by excessive and/or uncontrollable inflammation. In an aspect, a patient can refer to a subject that has been diagnosed with or is suspected of a disease or disorder characterized by excessive and/or uncontrollable inflammation and is seeking treatment or receiving treatment for the inflammation. In an aspect, a “patient” can refer to a subject afflicted with a disease or disorder characterized by excessive and/or uncontrollable inflammation. In an aspect, a patient can refer to a subject that has been diagnosed with or is suspected of having a disease or disorder characterized by excessive and/or uncontrollable inflammation. In an aspect, a patient can refer to a subject that has been diagnosed with or is suspected of having a disease or disorder characterized by excessive and/or uncontrollable inflammation and is seeking treatment or receiving treatment for an inflammatory disease or disorder.

As used herein, the phrase “identified to be in need of treatment,” or the like, refers to selection of a subject based upon need for treatment of a disease or disorder characterized by a disease or disorder characterized by excessive and/or uncontrollable inflammation. For example, a subject can be identified as having a need for treatment based upon an earlier diagnosis by a person of skill and thereafter subjected to treatment for a disease or disorder characterized by excessive and/or uncontrollable inflammation. In an aspect, the identification can be performed by a person different from the person making the diagnosis. In an aspect, the administration can be performed by one who performed the diagnosis.

The term “ex vivo” refers generally to activities that take place outside an organism such as experimentation, modification, differentiation, manipulation, and/or measurement done in or on living tissue in an artificial environment outside the organism. In an aspect, ex vivo experimentation, ex vivo modification, ex vivo differentiation, ex vivo manipulation, and/or ex vivo measurement can occur with a minimum alteration of the natural conditions. In an aspect, “ex vivo” can comprise living cells or tissues taken from an organism and cultured in a laboratory apparatus, usually under sterile conditions, and typically for a limited duration of time (e.g., a few hours or up to about 24 hours, up to about 48 hours, up to about 72 hours, up to about 96 hours, up to about 120 hours, up to about 144 hours, up to about 168 hours, or more depending on the circumstances and/or the desired characteristics. In an aspect, such tissues or cells can be collected, frozen, and later thawed for ex vivo treatment.

The term “in vivo” refers generally to activities that take place inside an organism.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of symptoms, complications, issues due to a subject's disease or disorder characterized by excessive and/or uncontrollable inflammation. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder characterized by excessive and/or uncontrollable inflammation) or to the level prior to the onset of a disease or disorder characterized by excessive and/or uncontrollable inflammation. Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the onset of inflammation. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the onset of inflammation. In an aspect, the inhibition or reduction can be 0-25%, 25-50%, 50-75%, or 75-100% as compared to native or control levels or to the subject's level prior to the onset of inflammation.

The words “treat” or “treating” or “treatment” include palliative treatment, that is, treatment designed for the relief of symptoms rather than the curing of a disease or disorder characterized by excessive and/or uncontrollable inflammation; preventative treatment, that is, treatment directed to minimizing or partially or completely inhibiting the development of a disease or disorder characterized by excessive and/or uncontrollable inflammation; and supportive treatment, that is, treatment employed to supplement another specific therapy directed toward the improvement of a disease or disorder characterized by excessive and/or uncontrollable inflammation. In an aspect, the terms cover any treatment of a subject, including a mammal (e.g., a human), and includes: (i) preventing the undesired physiological change and/or pathological condition from occurring in a subject that can be predisposed to a disease or disorder characterized by excessive and/or uncontrollable inflammation but has not yet been diagnosed as having it; (ii) inhibiting the physiological change and/or pathological condition (a disease or disorder characterized by excessive and/or uncontrollable inflammation); or (iii) relieving the physiological change and/or pathological condition, i.e., causing regression of a disease or disorder characterized by excessive and/or uncontrollable inflammation. For example, in an aspect, treating a disease or disorder can reduce the severity of an established a disease or disorder in a subject by 1%-100% as compared to a control (such as, for example, an individual not having a disease or disorder characterized by excessive and/or uncontrollable inflammation). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as a disease or disorder characterized by excessive and/or uncontrollable inflammation). For example, treating a disease or a disorder can reduce one or more symptoms of a disease or disorder in a subject by 1%-100% as compared to a control (such as, for example, an individual not having a disease or disorder characterized by excessive and/or uncontrollable inflammation). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., a disease or disorder characterized by excessive and/or uncontrollable inflammation). It is understood that treatment does not necessarily refer to a cure or complete ablation or eradication of a disease or disorder characterized by excessive and/or uncontrollable inflammation). However, in an aspect, treatment can refer to a cure or complete ablation or eradication of a disease or a disease or disorder characterized by excessive and/or uncontrollable inflammation).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing and/or treating and/or controlling inflammation or excessive inflammation is intended. The words “prevent” and “preventing” and “prevention” also refer to prophylactic or preventative measures for protecting or precluding a subject (e.g., an individual) not having inflammation related complication from progressing to that complication.

As used herein, the terms “administering” and “administration” refer to any method of providing one or more of the disclosed compositions (such as, for example, disclosed HDAC11-activated macrophages or disclosed HDAC11-activated M2 macrophages or a disclosed composition comprising HDAC11-activated macrophages or disclosed HDAC11-activated M2 macrophages). Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can be continuous or intermittent. Administration can comprise a combination of one or more route. In an aspect, disclosed HDAC11-activated macrophages or disclosed HDAC11-activated M2 macrophages or a disclosed composition comprising HDAC11-activated macrophages or disclosed HDAC11-activated M2 macrophages or any combination thereof can be concurrently and/or serially administered to a subject via multiple routes of administration. Various combinations of administration are known to the art.

By “determining the amount” is meant both an absolute quantification of a particular analyte (e.g., expression of a marker of M2 phenotype) or a determination of the relative abundance of a particular analyte (e.g., expression of a marker of M2 phenotype). The phrase includes both direct or indirect measurements of abundance or both. In an aspect, determining the amount can refer to measuring the expression of a marker of M2 phenotype.

As used herein, “modifying the method” can comprise modifying or changing one or more features or aspects of one or more steps of a disclosed method. In an aspect, a method can be altered by changing the amount of one or more of the disclosed compositions (e.g., a disclosed HDAC11-activated M2 macrophages, or a composition thereof, or a pharmaceutical formulation thereof) used in a disclosed method, or by changing the frequency of administration of one or more disclosed compositions (e.g., a disclosed HDAC11-activated M2 macrophages, or a composition thereof, or a pharmaceutical formulation thereof) in a disclosed method, by changing the duration of time that one or more disclosed compositions (e.g., a disclosed HDAC11-activated M2 macrophages, or a composition thereof, or a pharmaceutical formulation thereof) is administered in a disclosed method, or by substituting for one or more of the disclosed components and/or reagents with a similar or equivalent component and/or reagent.

As used herein, “concurrently” means (1) simultaneously in time, or (2) at different times during a common treatment schedule.

The term “contacting” as used herein refers to bringing one or more of the disclosed compositions (e.g., a disclosed HDAC11 inhibitor) together with a target area or intended target area (e.g., a population of macrophages) in such a manner that the disclosed compositions can exert an effect on the intended target or targeted area either directly or indirectly. A target area or intended target area can be one or more cells (e.g., M0 or naïve macrophages) and/or one or more tissues having inflammation, or any combination thereof. In an aspect, a target area or intended target area can be any cell or any organ infected by a disease or disorder (such as inflammation). In an aspect, a target area or intended target area can be any organ, tissue, or cells that are affected by a disease or disorder characterized by inflammation.

As used herein, “determining” can refer to measuring or ascertaining the presence and severity of a disease or disorder, such as, for example, characterized by excessive and/or uncontrollable inflammation. “Determining” can refer to measuring or ascertaining an expression level of a protein or gene of interest. “Determining” can refer to measuring or ascertaining the reprogramming of M0 or naïve macrophages. “Determining” can refer to ascertaining or measuring some type of neurologic, physiologic, and/or metabolic function and/or response.

Methods and techniques used to determine the presence and/or severity of a disease or disorder characterized by excessive and/or uncontrollable inflammation are typically known to the medical arts. For example, the art is familiar with the ways to identify and/or diagnose the presence, severity, or both of a disease or disorder characterized by excessive and/or uncontrollable inflammation. Methods can be based on objective and/or subjective means.

As used herein, “effective amount” and “amount effective” can refer to an amount that is sufficient to achieve the desired result such as, for example, the treatment and/or prevention of a disease or disorder characterized by excessive and/or uncontrollable inflammation. As used herein, the terms “effective amount” and “amount effective” can refer to an amount that is sufficient to achieve the desired an effect on an undesired condition (e.g., a disease or disorder characterized by excessive and/or uncontrollable inflammation). For example, a “therapeutically effective amount” refers to an amount that is sufficient to achieve the desired therapeutic result or to have an effect on undesired symptoms, but is generally insufficient to cause adverse side effects.

In an aspect, “therapeutically effective amount” means an amount of the disclosed composition that (i) treats a disease or disorder characterized by excessive and/or uncontrollable inflammation, (ii) attenuates, ameliorates, or eliminates one or more symptoms associated with a disease or disorder characterized by excessive and/or uncontrollable inflammation, or (iii) delays the onset of one or more symptoms of a disease or disorder characterized by excessive and/or uncontrollable inflammation. The specific therapeutically effective dose level for any particular patient will depend upon a variety of factors including the disease or disorder characterized by excessive and/or uncontrollable inflammation being treated; the disclosed compositions employed; the disclosed methods employed; the age, body weight, general health, sex and diet of the patient; the time of administration; the route of administration; the rate of excretion of the disclosed compositions employed; the duration of the treatment; drugs used in combination or coincidental with the disclosed compositions employed, and other like factors well known in the medical arts. For example, it is well within the skill of the art to start doses of the disclosed compositions at levels lower than those required to achieve the desired therapeutic effect and to gradually increase the dosage until the desired effect is achieved. If desired, then the effective daily dose can be divided into multiple doses for purposes of administration. Consequently, a single dose of the disclosed HDAC11-activated M2 macrophages, the disclosed compositions, disclosed pharmaceutical formulations, disclosed therapeutic agents, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a sign or symptom associated with a disease or disorder characterized by excessive and/or uncontrollable inflammation.

Disclosed are the components to be used to prepare the disclosed compositions, disclosed viral vectors, disclosed pharmaceutical formulations, disclosed therapeutic agents, or a combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Histone Deacetylases

The removal of acetyl groups from e-lysine residues in proteins connected to condensed chromatin structures that inhibit gene transcription is catalyzed by a class of enzymes called histone deacetylases (HDACs). Mammals currently contain 18 HDACs that are classified into two families: (i) the Zn2+-dependent or classical HDACs, and (ii) the nicotinamide adenine dinucleotide (NAD+)-dependent HDACs or sirtuins (SIRT). According to the homology of their catalytic domains, classical HDACs are further split into three classes: Class I, Class II, and Class IV HDACs. Class I HDACs include HDAC1, HDAC2, HDAC3, and HDAC8, whereas Class II HDACs include HDAC4, HDAC5, HDAC6, HDAC8, HDAC9, and HDAC10, and Class IV HDACs include HDAC11.

HDAC11, the solitary member of Class IV HDAC, contains an open reading frame encoding a 347-residue protein and shares sequence homology with both class I and class II HDAC proteins in the catalytic core regions. HDAC11 is highly conserved, even in invertebrates and plants, and combines with other HDACs to form functional complexes. Although HDAC11 structure has still not been discovered, it has been effectively modeled from HDAC8 structure. HDAC11 can be degraded by the proteasome system and has an unstable half-life at around four hours. While most class I-III HDACs are involved in deacetylating their substrate, HDAC11 has defattyacylase activity in addition to its deacetylase activity. In fact, as the only HDAC member that has a clear predilection for the removal of long-acyl instead of acetyl groups, HDAC11 is the family's most effective fatty deacetylase. It has been reported that the efficiency of HDAC11 defattyacylase activity is greater than 10,000 times its deacetylase activity. The activation of HDAC11 can be triggered by physiologic levels of free fatty acids and their metabolites.

HDAC11 can also be identified as HGNC No. 19086, NCBI Entrez Gene No. 79885, Ensembl No. ENSG00000163517, OMIM No. 607226, and UniProtKB/Swiss-Prot. No. Q96DB2.

### C. Macrophages

Macrophages constitute a heterogeneous cell population representing innate immunity. Macrophages have been identified in all tissues and their chief competences are phagocytic activity and antigen presentation. Macrophages continuously monitor their microenvironments for the presence of pathogens, unfit cells, debris, and toxic metabolites, and release a variety of active substances including growth factors and cytokines. Human macrophages express a number of markers including CD14, CD16, CD68, CD163, CD11b, CD86, and CD206.

By contrast with many other cell types, macrophages cannot be traced to a single origin. The modern concept of macrophage origin includes three developmental sources of these cells, which correspond to the three generations of hematopoietic stem cells. The first generation develops from the extraembryonic yolk sac posterior plate mesoderm in the blood islands. These cells apparently give rise to the microglia of the central nervous system. The second wave of hematopoietic progenitors, which develops from the yolk sac hematogenic endothelium, is called crythro-myeloid precursors. After the onset of blood circulation, these cells colonize the embryonic liver. Erythro-myeloid precursors give rise to granulocyte, monocyte, and macrophage lineages. The third generation of hematopoietic cells is derived from endothelium in the aorto-gonado-mesonephral zone; these cells colonize the fetal liver where they establish hematopoiesis and the red bone marrow where they produce the bone marrow hematopoietic stem cell lineages. During embryogenesis, macrophages in organs are predominantly represented by cells of the second and third generations, while postnatal development is marked by increasing percentage of macrophages derived from the third generation.

The most-discussed current classification of macrophages is based on the M1/M2 paradigm, which is related to their pro- and anti-inflammatory properties. The M1/M2 paradigm states that macrophages can switch their phenotypes from the pro-inflammatory M1 to the anti-inflammatory M2 and vice versa, depending on the needs of the microenvironment, or maintain the naïve state M0 in the absence of external signals. The classical activation of macrophages is promoted by lipopolysaccharide (LPS), interferon gamma (IFN-γ), and granulocyte-macrophage colony-stimulating factor (GM-CSF). The resulting classical (M1) phenotype is characterized by expression of TLR-2, TLR-4, CD80, and CD86. M1 macrophages secrete pro-inflammatory cytokines (interleukins IL-1β, IL-12, IL-18 and IL-23, and tumor necrosis factor alpha TNF-α) that modulate the Th1-mediated antigen-specific inflammatory reactions. M1 macrophages have also been demonstrated to enhance the expression of inducible nitric oxide synthase (NOS2 or iNOS) to facilitate the production of NO from L-arginine.

TABLE 1
Human Macrophage Phenotype Markers.

| | M1 | M2 |
|---|---|---|
| CD163 | ↓ | ↑ |
| Mannose receptor (CD206) | ↓ | ↑ |
| AMACI (CCL-18) | ↓ | ↑ |
| IL-10 | ↓ | ↑ |
| TNFα | ↑ | ↓ |
| iNOS | ↑ | ↓ |
| IL-12 | ↑ | ↓ |
| IL-6 | ↑ | ↓ |
| MCP-1 (CCL2) | ↑ | ↓ |

Activation of macrophages towards M2 phenotypes can be induced by antigen-antibody complexes, invading helminths, complement system components, apoptotic cells, interleukins (IL-4, IL-13, and IL-10), and transforming growth factor beta (TGF-β). Activation with these inducers drives macrophages towards the increased secretion of IL-10 and reduced secretion of IL-12 typical of the M2 phenotypes. M2 macrophages show diverse gene expression signatures, and distinct M2a, M2b, M2c, and M2d macrophage subpopulations have been identified by transcriptome analysis. The corresponding M2 phenotypes are generally characterized by high levels of mannose receptor CD206 and scavenger receptor CD163. Arginase 1, which converts arginine into ornithine—an important building block for collagen synthesis, is a relevant marker of M2 macrophage polarization in rats and mice.

To date, the most common macrophage sources are bone marrow, spleen, and peritoneal cavity. Compared to bone marrow-derived macrophages (BMDMs) and splenic macrophages (SPMs), peritoneal macrophages (PMs) appear to be more mature with higher expression of inducible cytokines and are more stable in their functionality and phenotype. Therefore, PMs isolated from the peritoneal cavity are the common source of macrophages for various in vitro assays, including stimulation with Toll-like receptor (TLR) ligands, cell signaling assay, phagocytosis, cytokine production, chemokine production, and toxicology study.

PMs are the major cell type of peritoneal cells (more than 30%). PMs can be classified into classically activated macrophages (M1) and alternatively activated macrophages (M2) following stimulation. This classification method is mainly based on cell phenotype and function. Notably, M1-polarized PMs have long been identified to play an important role in host defense, which express Th1 cytokines and inflammatory cytokines, including tumor necrosis factor-α (TNF-α), interleukin-2 (IL-2), and interferon-γ (IFN-γ). M2-polarized PMs predominantly express a large amount of Th2 cytokines and anti-inflammatory cytokines, including IL-4, IL-13, IL-10, and transforming growth factor-β (TGF-β), thereby downregulating inflammatory processes.

Accumulating studies have demonstrated that PMs in the peritoneal cavity strongly express CD206 mRNA, which is the characteristic phenotype of M2-polarized macrophages. Therefore, M2-polarized PMs are the major composition of PMs. Additionally, PMs can be classified into another two subsets based on morphology: large PMs and small PMs. These two macrophage subsets exhibit distinct origin and morphology. On the one hand, large PMs have been characterized as fetal-originated tissue resident macrophages with a high level of F4/80 and a low level of major histocompatibility complex II (MHC-II). Under steady condition, large PMs compose the major population of PMs and are characterized with high expression of transcription factor GATA6. It has been proven that the GATA6 expressed in large PMs selectively regulates the level of aspartoacylase and therefore control the survival, differentiation, and metabolism of resident PMs. On the other hand, small PMs appear to be generated from embryogenic precursors with a low level of F4/80 and a high level of MHC-II. It has been reported that the PMs have a cross-talk between T lymphocytes, which are enriched in IL-17 receptor A and express a proangiogenic gene profile, and therefore directly promote ovarian cancer cell proliferation.

### D. Immunity

The human immune system is a complex and powerful defense mechanism. The primary function of the immune system is to defend the body from pathogens, which are disease-causing organisms such as viruses and bacteria. Tissues, cells, and proteins in the immune system work together to achieve this function.

To fight infections, the immune system must be able to identify pathogens. Pathogens have molecules called antigens on their surface. Antigens provide a unique signature for the pathogen that enables immune system cells to recognize different pathogens and distinguish pathogens from the body's own cells and tissues. When a pathogen gets into the body, the immune system reacts in two ways.

The innate immune response is a rapid reaction. Innate immune cells recognize certain molecules found on many pathogens. These cells also react to signaling molecules released by the body in response to infection. Through these actions, innate immune cells quickly begin fighting an infection. This response results in inflammation. The cells involved in this reaction can kill pathogens and can also help activate cells involved in adaptive immunity.

The adaptive immune response is slower than the innate response but is better able to target specific pathogens. There are two main cell types involved in this response: T cells and B cells. Some T cells kill pathogens and infected cells. Other T cells help control the adaptive immune response. The main function of B cells is to make antibodies against specific antigens. Antibodies, also known as immunoglobulins, are proteins that attach themselves to pathogens. This signals immune cells to destroy the pathogen.

It takes time for T and B cells to respond to the new antigens when a pathogen causes an infection. Once exposed to the pathogen, these cells develop a memory for the pathogen so that they are ready for the next infection. As part of the adaptive immune response, some T and B cells change into memory cells. Memory cells mostly stay in the lymph nodes and the spleen and “remember” particular antigens. If a person becomes infected with the same pathogen again, these cells are able to quickly and vigorously begin fighting the infection.

### E. Adaptive Cell Therapy

As used herein, adaptive cell therapy can involve the introduction of cells into a subject in need of treatment for a specific disease, disorder, or condition (such as an inflammatory disease (e.g., AD or AIF), or a chronic wound, or some other condition having inflammation). Specifically, as described herein, naïve or M0 macrophages can be obtained from a subject, activated using one or more disclosed HDAC11 inhibitors, polarized into an anti-inflammatory M2 phenotype, and then returned to the same subject via local or system administration.

### F. Autoimmune Diseases and Disorders

Autoimmune disease (AD) originates in the adaptive immune system. AD occurs when adaptive immune cells lose their ability to maintain self-tolerance of human cells. As a result, the immune system begins attacking healthy tissues as though they were infectious agents. Some ADs are organ-specific, whereas others can affect multiple parts of the body. Recurrent fever and chronic inflammation can be attributed to autoimmune disease. However, many possible symptoms are unique to AD, including hair loss, dry mouth, temperature sensitivity, muscle weakness, reproductive issues, and more.

A variety of conditions including environment, hormones, injury, infection, and genes can all play a role in initiating autoimmunity. AD is thought to be triggered by a combination of multiple factors not limited to genetics. Although the exact causes remain unknown, researchers believe that a variety of conditions including environment, hormones, injury, infection, and genes could all play a role in initiating autoimmunity. Autoimmune conditions are quite common. Current data estimates that ADs affect at least 14 million people in the U.S. alone. There are 80-100 known autoimmune and autoimmune-related conditions, including Hashimoto's thyroiditis, Type 1 diabetes, Sjögren's, and celiac disease. Gender prevalence in AD is well documented. In fact, around 80% of those diagnosed are women. While ADs can affect anyone at any age, the majority tend to appear during adulthood.

### G. Autoinflammatory Diseases and Disorder

Most distinctly, autoinflammatory (AIF) diseases only involve the innate immune system. AIFs occur when innate immune cells are activated without an infection or injury being present. This mishap kickstarts the release of cytokines and other immune responses, causing fever and inflammation. Recurring episodes of high fever are the primary symptom of AIF. Similar to some autoimmune diseases, autoinflammatory symptoms can be widespread, affecting the joints, gastrointestinal tract, skin, eyes, and internal organs.

Autoinflammatory diseases are caused by mutations in the genes that control and regulate innate immune cells. AIFs include but are not limited to Familial Mediterranean Fever (FMF), hyper IgD syndrome (HIDS), TNF receptor-associated periodic syndrome (TRAPS), cryopyrin associated periodic syndromes (CAPS), and Blau syndrome and pyogenic sterile arthritis pyoderma gangrenosum and acne syndrome (PAPA). Some AIFs are monogenic because they are caused by a singular known genetic defect. Because these diseases originate in the genes, they can run in families. Autoinflammatory syndromes are very rare. Presently, there is no known gender prevalence. Unlike autoimmune disease, the majority of AIFs develop during childhood. Due to the rarity of these diseases and their close appearance to other illnesses, diagnosis is often delayed. Several AIFs are known for their higher frequencies within certain ethnicities. As such, age of onset and familial history, in addition to clinical observations, can be helpful in diagnosing these rare conditions. In some cases, testing for gene mutations is used to confirm a diagnosis of AIF.

### H. Inflammatory Diseases and/or Disorders

Inflammation can refer to a biological response to stimuli interpreted by the body to have a potentially harmful effect. Inflammation is a normal, healthy response to injury, infections, or certain other medical conditions. An inflammatory disorder, however, is where the immune system causes inflammation by mistakenly attacking your body's own cells or tissues. There are a number of ways that the immune system can go wrong and cause inflammation.

A disclosed inflammatory disease and/or disorder can be Encephalitis, Myelitis, Meningitis, Arachnoiditis, Neuritis, Dacryoadenitis, Scleritis, Episcleritis, Keratitis, Retinitis, Chorioretinitis, Blepharitis, Conjunctivitis, Uveitis, Otitis externa, Otitis media, Labyrinthitis, Mastoiditis, Carditis, Endocarditis, Myocarditis, Pericarditis, Vasculitis, Arteritis, Phlebitis, Capillaritis, Sinusitis, Rhinitis, Pharyngitis, Laryngitis Tracheitis, Bronchitis, Bronchiolitis, Pneumonitis, Pleuritis, Mediastinitis, Stomatitis, Gingivitis, Gingivostomatitis, Glossitis, Tonsillitis, Sialadenitis/Parotitis, Cheilitis, Pulpitis, Gnathitis, Esophagitis, Gastritis, Gastroenteritis, Enteritis, Colitis, Enterocolitis, Duodenitis, Ileitis, Caccitis, Appendicitis, Proctitis, Hepatitis, Ascending cholangitis, Cholecystitis, Pancreatitis, Peritonitis, Dermatitis, Folliculitis, Cellulitis, Hidradenitis, Arthritis, Dermatomyositis, Myositis, Synovitis/Tenosynovitis, Bursitis, Enthesitis, Fasciitis, Capsulitis, Epicondylitis, Tendinitis, Panniculitis, Osteochondritis, Spondylitis, Periostitis, Chondritis, Nephritis, Glomerulonephritis, Pyelonephritis, Ureteritis, Cystitis, Urethritis, Oophoritis, Salpingitis, Endometritis, Parametritis, Cervicitis, Vaginitis, Vulvitis, Mastitis, Orchitis, Epididymitis, Prostatitis, Seminal vesiculitis, Balanitis, Posthitis, Balanoposthitis, Chorioamnionitis, Funisitis, Omphalitis, Insulitis, Hypophysitis, Thyroiditis, Parathyroiditis, Adrenalitis, Lymphangitis, Lymphadenitis, or any combination thereof.

Chronic wounds are wounds that fail to restore intact anatomic and physiological function through the normal repair process, and mainly include diabetic foot ulcers, venous leg ulcers, and pressure sores. With the aging of the population, the number of chronic wound patients increases year by year at present. Due to the long healing period of chronic wounds, patients often need to be hospitalized for a long time, which consumes a large number of medical resources and puts a great strain on medical systems and patient families. Meanwhile, the healing of chronic wounds is difficult, and doctors need to adjust therapy in time according to the state of the wounds in the treatment and nursing process so as to shorten the healing time. In wound assessment, changes in physical parameters (area) directly reflect the healing process of the wound, and accurate measurement of wound physical parameters is the most critical task in wound care. However, at present, in clinical practice, contact measurement is mainly performed on patients by using a ruler or a transparent mark, and the result is subjectively influenced by a measurer, and meanwhile, pain is brought to the patients and the infection risk is increased. The existing non-contact measuring equipment is often expensive and poor in usability. Therefore, the efficient and accurate automatic chronic wound measuring equipment can effectively solve the problem of clinical wound assessment and improve the efficiency of chronic wound care and treatment. Chronic wounds, typically diabetic ulcers, preceded 85% of amputations. Some chronic wounds can take decades to heal, thus contributing to secondary conditions such as depression, and can ultimately lead to isolation and family distress. The five-year mortality rate after developing a diabetic ulcer is approximately 40%; therefore, proper diagnosis and treatment of wounds and management of comorbidities are imperative. Arterial Ulcer Arterial ulcers are typically located on the distal extremities and may be deep, with tendon or bone exposed. As with venous ulcers, the initial workup should include ankle-brachial index (ABI) measurement and palpation of pulses. An ABI less than 0.8 may be a sign of arterial disease, and an ABI greater than 1.2 is consistent with noncompressible vessels. Both require further vascular assessment, which may include arterial Doppler/duplex ultrasonography, segmental limb pressures, pulse volume recording, skin perfusion pressure, or transcutaneous oximetry. Referral to a specialist should be considered if vascular test results suggest poor perfusion. Venous Ulcer Venous ulcers are the most common type of chronic wound. They are typically shallow and located on the medial supramalleolar aspect of the lower extremity. There may be classic signs of venous hypertension, including edema, hemosiderin staining, and lipodermatosclerosis. Workup should include assessment of arterial status with the ankle-brachial index (ABI) and palpation of pulses to rule out mixed arterial and venous disease and to ensure adequate perfusion before compression. If necessary, further workup with venous duplex ultrasonography with reflux and/or arterial duplex ultrasonography can help with the diagnosis. Diabetic Ulcers Diabetic ulcers are the most common cause of lower extremity amputation. Early intervention and management are essential given the high mortality rate after amputation. Diabetic foot ulcers are caused by a combination of underlying neuropathy, peripheral arterial disease, and structural deformities that cause increased pressure on affected areas of the foot. Diabetes mellitus affects sensory, motor, and autonomic nerve function. The combined effects result in structural deformities of the foot; dry, poorly hydrated integument; and an inability to detect pain and repetitive injury. Diabetes also causes higher rates of atherosclerotic disease. Diabetic ulcers are typically located on the toes or the plantar aspect of the metatarsal heads. They may have the characteristic crater-like appearance, be covered in eschar or necrotic tissue at the wound bed and have exposed deep structures including tendon and bone. These ulcers can be shallow or deep and are usually surrounded by a thick ring of callus. Workup should include palpation of pedal pulses, ABI measurement, and assessment for neuropathy with Semmes-Weinstein monofilament. Imaging is often required to rule out deeper infection such as osteomyelitis. Patients with diabetes often have calcified, noncompressible arteries that result in an abnormally high ABI. A more reliable test in these patients is the toe-brachial index because the toc arteries rarely calcify.

### I. Compositions for Use in the Disclosed Methods

### 1. Activated Macrophages

Disclosed herein are HDAC11-activated macrophages. Disclosed herein are HDAC11-activated M2 macrophages. Disclosed herein are HDAC11-activated macrophages made by a method disclosed herein. Disclosed herein are HDAC11-activated M2 macrophages made by a method disclosed herein. Disclosed herein are HDAC11-activated macrophages made by a method comprising contacting a population of naïve macrophages with one or more HDAC11 inhibitors. Disclosed herein are HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein are HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein are HDAC11-activated macrophages made by a method comprising contacting a population of polarized macrophages with one or more HDAC11 inhibitors. Disclosed herein are HDAC11-activated macrophages made by a method comprising contacting a population of polarized macrophages with one or more HDAC11 inhibitors, wherein the macrophages are polarized towards an anti-inflammatory phenotype. Disclosed herein are HDAC11-activated macrophages made by a method comprising contacting a population of polarized macrophages with one or more HDAC11 inhibitors, wherein the macrophages are polarized towards a M2 phenotype.

Disclosed herein are cryopreserved HDAC11-activated macrophages. Disclosed herein are cryopreserved HDAC11-activated M2 macrophages. Disclosed herein are cryopreserved HDAC11-activated macrophages made by a method disclosed herein. Disclosed herein are cryopreserved HDAC11-activated M2 macrophages made by a method disclosed herein. Disclosed herein are cryopreserved HDAC11-activated macrophages made by a method comprising contacting a population of naïve macrophages with one or more HDAC11 inhibitors. Disclosed herein are cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein are cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein are cryopreserved HDAC11-activated macrophages made by a method comprising contacting a population of polarized macrophages with one or more HDAC11 inhibitors. Disclosed herein are cryopreserved HDAC11-activated macrophages made by a method comprising contacting a population of polarized macrophages with one or more HDAC11 inhibitors, wherein the macrophages are polarized towards an anti-inflammatory phenotype. Disclosed herein are cryopreserved HDAC11-activated macrophages made by a method comprising contacting a population of polarized macrophages with one or more HDAC11 inhibitors, wherein the macrophages are polarized towards a M2 phenotype.

In an aspect, disclosed HDAC11-activated macrophages can enhance and/or improve the M2 phenotype of the macrophages upon polarization. In an aspect, disclosed HDAC11-activated macrophages can enhance and/or improve the anti-inflammatory phenotype of the polarized M2 macrophages. In an aspect, HDAC11-activated macrophages can be cryopreserved prior to polarization or can be cryopreserved after polarization to an M2 phenotype. In an aspect, disclosed HDAC11-activated macrophages or disclosed HDAC11-activated M2 macrophages can be used to treat a subject can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, disclosed HDAC11-activated macrophages or disclosed HDAC11-activated M2 macrophages can be used to treat a subject having one or more foot ulcers. In an aspect, disclosed HDAC11-activated macrophages or disclosed HDAC11-activated M2 macrophages can be used to treat subject having diabetes. In an aspect, disclosed HDAC11-activated macrophages or disclosed HDAC11-activated M2 macrophages can be used to treat a subject that is the recipient of one or more solid organ transplants. In an aspect, disclosed HDAC11-activated macrophages or disclosed HDAC11-activated M2 macrophages can be used to treat a subject that has irritable bowel disease (IBD).

In an aspect, disclosed HDAC11-activated macrophages or disclosed HDAC11-activated M2 macrophages can be used to treat a subject having one or more inflammatory diseases. In an aspect, disclosed HDAC11-activated macrophages or disclosed HDAC11-activated M2 macrophages can be used to treat a subject having one or more skin inflammatory diseases. In an aspect, disclosed HDAC11-activated macrophages or disclosed HDAC11-activated M2 macrophages can be used to treat a subject having suffered one or more burns.

In an aspect, disclosed HDAC11-activated macrophages or disclosed HDAC11-activated M2 macrophages can be used to treat a subject having incurred an ischemic injury. In an aspect, a disclosed ischemic injury can affect the subject's brain and/or the subject's heart. For example, in an aspect, a disclosed subject can have suffered one or more myocardial infarctions, or a disclosed subject can have suffered one or more transient ischemic attacks. In an aspect, disclosed HDAC11-activated macrophages or disclosed HDAC11-activated M2 macrophages can be used to treat a subject having an autoinflammatory disease (AIF). AIFs are discussed infra. In an aspect, disclosed HDAC11-activated macrophages or disclosed HDAC11-activated M2 macrophages can be used to treat a subject having an autoimmune disease (AD). AIDs are discussed infra.

In an aspect, disclosed HDAC11-activated macrophages or disclosed HDAC11-activated M2 macrophages can be used to treat an adult, a teenager, an adolescent, a child, a toddler, a baby, or an infant. In an aspect, a subject can have received treatment for one or more disclosed AIFs or ADs. In an aspect, a subject can be treatment-naïve.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, about 50 nM to about 25 UM of a disclosed HDAC11 inhibitor can polarize resting or M0 macrophages into a M2 phenotype or an anti-inflammatory phenotype.

In an aspect, disclosed HDAC11-activated macrophages or disclosed HDAC11-activated M2 macrophages can be administered to a subject using intravenous administration. In an aspect, disclosed HDAC11-activated macrophages or disclosed HDAC11-activated M2 macrophages can be administered to a subject using systemic administration or local administration.

In an aspect, disclosed HDAC11-activated macrophages or disclosed HDAC11-activated M2 macrophages can be administered to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect, disclosed HDAC11-activated macrophages or disclosed HDAC11-activated M2 macrophages can be administered to directly to a target site within a subject. For example, disclosed HDAC11-activated macrophages or disclosed HDAC11-activated M2 macrophages can be locally delivered by direct injection into an affected body system (such as one, for example, having inflammation). Local delivery or local administration can also include topical applications or localized injection techniques such as intramuscular, subcutaneous, and/or intradermal injection of disclosed HDAC11-activated macrophages or disclosed HDAC11-activated M2 macrophages.

In an aspect, local administration of disclosed HDAC11-activated macrophages or disclosed HDAC11-activated M2 macrophages can comprise delivery to one or more body systems in need thereof. In an aspect, local administration or local delivery of disclosed HDAC11-activated macrophages or disclosed HDAC11-activated M2 macrophages can comprise a transdermal administration, administration via implant, administration via suppository, implantation via a biodegradable polymer or matrix, implantation via a biodegradable hydrogel, or any combination thereof. In an aspect, when administered, the disclosed HDAC11-activated macrophages or disclosed HDAC11-activated M2 macrophages can diminish and/or decrease inflammation in one or more of the subject's body systems. In an aspect, the diminishment and/or decrease in inflammation in one or more of the subject's body systems can be measured objectively and/or subjectively.

In an aspect, when administered, the disclosed HDAC11-activated macrophages or disclosed HDAC11-activated M2 macrophages can diminish and/or decrease the pathology of the subject's disease or disorder. In an aspect, when administered, the disclosed HDAC11-activated macrophages or disclosed HDAC11-activated M2 macrophages can diminish and/or decrease the subject's symptoms. In an aspect, when administered, the disclosed HDAC11-activated macrophages or disclosed HDAC11-activated M2 macrophages can improve and/or enhance the subject's quality of life. In an aspect, the subject's quality of life can be measured objectivity and/or subjectively. In an aspect, when administered, the disclosed HDAC11-activated macrophages or disclosed HDAC11-activated M2 macrophages can prevent the rejection of one or more transplanted organs. In an aspect, when administered, the disclosed HDAC11-activated macrophages or disclosed HDAC11-activated M2 macrophages can spare one or more transplanted organs from rejection. In an aspect, when administered, the disclosed HDAC11-activated macrophages or disclosed HDAC11-activated M2 macrophages can improve and/or enhance the healing of the subject's one or more chronic wounds.

In an aspect, when administered, the disclosed HDAC11-activated macrophages or disclosed HDAC11-activated M2 macrophages can improve and/or stimulate angiogenesis, innervation, cellular migration, or any combination thereof. In an aspect, when administered, the disclosed HDAC11-activated macrophages or disclosed HDAC11-activated M2 macrophages can produce signs of cellular regeneration and/or cellular repair in one or more body systems (such as, for example, the cardiovascular system or the central nervous system and/or peripheral nervous system). In an aspect, when administered, the disclosed HDAC11-activated macrophages or disclosed HDAC11-activated M2 macrophages can produce signs of cellular regeneration and/or cellular repair in one or more body systems (such as, the integumentary system or the digestive system). In an aspect, when administered, the disclosed HDAC11-activated macrophages or disclosed HDAC11-activated M2 macrophages can diminish and/or decrease inflammation in one or more of the subject's body systems. Body systems are disclosed infra. In an aspect, a decrease in inflammation in one or more of the subject's body systems can be measured objectively and/or subjectively. In an aspect, when administered, the disclosed HDAC11-activated macrophages or disclosed HDAC11-activated M2 macrophages can further diminish and/or decrease the pathology of the subject's disease or disorder, can further diminish and/or decrease the subject's symptoms, can further improve and/or enhance the subject's quality of life, or any combination thereof. In an aspect, quality of life can be measured objectivity and/or subjectively.

In an aspect, when administered, the disclosed HDAC11-activated macrophages or disclosed HDAC11-activated M2 macrophages can prevent the rejection of one or more transplanted organs in the subject, can minimize the risk of rejection of one or more transplanted organs in the subject, can improve and/or enhance the healing of one or more chronic wounds in the subject, can improve and/or enhance healing of one or more chronic wounds, can improve and/or enhance angiogenesis, innervation, cellular migration, or any combination thereof in and/or around one or more chronic wounds in the subject, can improve and/or enhance cellular regeneration and/or cellular repair or one or more of the subject's affected body systems, can improve and/or enhance cellular regeneration and/or cellular repair in the subject's central nervous system and/or peripheral nervous system, can improve and/or enhance cellular regeneration and/or cellular repair in the subject's integumentary system, can improve and/or enhance cellular regeneration and/or cellular repair in the subject's digestive system, or any combination thereof. In an aspect, disclosed HDAC11-activated macrophages or disclosed HDAC11-activated M2 macrophages can be formulated as a composition. In an aspect, disclosed HDAC11-activated macrophages or disclosed HDAC11-activated M2 macrophages can be formulated as a composition with one or more therapeutic agents and/or active agents. In an aspect, disclosed therapeutic agents and/or active agents can comprise (i) one or more biologically active agents, (ii) one or more pharmaceutically active agents, (iii) one or more immune-based therapeutic agents, (iv) one or more clinically approved agents, (v) one or more proteasome inhibitors, (vi) one or more immunosuppressives or immunosuppressive agents, or (vii) any combination thereof. In an aspect, an immunosuppressive agent can be anti-thymocyte globulin (ATG), cyclosporine (CSP), mycophenolate mofetil (MMF), or a combination thereof.

In an aspect, disclosed HDAC11-activated macrophages or disclosed HDAC11-activated M2 macrophages can be formulated as a composition for systemic administration or for local administration. In an aspect, disclosed HDAC11-activated macrophages or disclosed HDAC11-activated M2 macrophages can be formulated as a composition with one or more therapeutic agents and/or active agents for systemic administration or for local administration. In an aspect, disclosed HDAC11-activated macrophages or disclosed HDAC11-activated M2 macrophages can be formulated as a composition. In an aspect, disclosed HDAC11-activated macrophages or disclosed HDAC11-activated M2 macrophages can be formulated as a composition with one or more therapeutic agents and/or active agents. In an aspect, a disclosed composition comprising disclosed HDAC11-activated macrophages or disclosed HDAC11-activated M2 macrophages can further comprise one or more disclosed excipients (discussed infra) and/or one or more disclosed pharmaceutically acceptable carriers (discussed infra).

### 2. Formulations

Disclosed herein is a pharmaceutical formulation comprising one or more disclosed HDAC11-activated M2 macrophages, and one or more pharmaceutically acceptable carrier. Disclosed herein is a pharmaceutical formulation comprising one or more disclosed composition comprising HDAC11-activated macrophages, and one or more pharmaceutically acceptable carrier. Disclosed herein is a pharmaceutical formulation comprising one or more disclosed cryopreserved HDAC11-activated M2 macrophages, and one or more pharmaceutically acceptable carrier. Disclosed herein is a pharmaceutical formulation comprising one or more disclosed composition comprising cryopreserved HDAC11-activated macrophages, and one or more pharmaceutically acceptable carrier.

In an aspect, a disclosed pharmaceutical formulation can further comprise one or more excipients. In an aspect, a disclosed excipient can refer to an inert substance that is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, a disclosed pharmaceutically acceptable carrier can refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their case of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can enhance and/or improve the M2 phenotype of the macrophages upon polarization. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can enhance and/or improve the anti-inflammatory phenotype of the polarized M2 macrophages. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be cryopreserved prior to polarization or can be cryopreserved after polarization to an M2 phenotype. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having one or more foot ulcers. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat subject having diabetes. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject that is the recipient of one or more solid organ transplants. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject that has irritable bowel disease (IBD).

In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having one or more inflammatory diseases. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having one or more skin inflammatory diseases. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having suffered one or more burns. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having incurred an ischemic injury. In an aspect, a disclosed ischemic injury can affect the subject's brain and/or the subject's heart. For example, in an aspect, a disclosed subject can have suffered one or more myocardial infarctions, or a disclosed subject can have suffered one or more transient ischemic attacks. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having an autoinflammatory disease (AIF). AIFs are discussed infra. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat a subject having an autoimmune disease (AD). AIDs are discussed infra. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be used to treat an adult, a teenager, an adolescent, a child, a toddler, a baby, or an infant. In an aspect, a subject can have received treatment for one or more disclosed AIFs or ADs. In an aspect, a subject can be treatment-naïve.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, about 50 nM to about 25 μM of a disclosed HDAC11 inhibitor can polarize resting or M0 macrophages into a M2 phenotype or an anti-inflammatory phenotype.

In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be administered to a subject using intravenous administration. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be administered to a subject using systemic administration or local administration. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be administered to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be administered to directly to a target site within a subject. For example, disclosed HDAC11-activated macrophages or disclosed HDAC11-activated M2 macrophages can be locally delivered by direct injection into an affected body system (such as one, for example, having inflammation). Local delivery or local administration can also include topical applications or localized injection techniques such as intramuscular, subcutaneous, and/or intradermal injection of disclosed HDAC11-activated macrophages or disclosed HDAC11-activated M2 macrophages. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can comprise delivery to one or more body systems in need thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can comprise a transdermal administration, administration via implant, administration via suppository, implantation via a biodegradable polymer or matrix, implantation via a biodegradable hydrogel, or any combination thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can diminish and/or decrease inflammation in one or more of the subject's body systems. In an aspect, the diminishment and/or decrease in inflammation in one or more of the subject's body systems can be measured objectively and/or subjectively. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can diminish and/or decrease the pathology of the subject's disease or disorder. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can diminish and/or decrease the subject's symptoms. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can improve and/or enhance the subject's quality of life. In an aspect, the subject's quality of life can be measured objectivity and/or subjectively. In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can prevent the rejection of one or more transplanted organs. In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can spare one or more transplanted organs from rejection. In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can improve and/or enhance the healing of the subject's one or more chronic wounds.

In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can improve and/or stimulate angiogenesis, innervation, cellular migration, or any combination thereof. In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can produce signs of cellular regeneration and/or cellular repair in one or more body systems (such as, for example, the cardiovascular system or the central nervous system and/or peripheral nervous system). In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can produce signs of cellular regeneration and/or cellular repair in one or more body systems (such as, the integumentary system or the digestive system). In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can diminish and/or decrease inflammation in one or more of the subject's body systems. Body systems are disclosed infra. In an aspect, a decrease in inflammation in one or more of the subject's body systems can be measured objectively and/or subjectively. In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can further diminish and/or decrease the pathology of the subject's disease or disorder, can further diminish and/or decrease the subject's symptoms, can further improve and/or enhance the subject's quality of life, or any combination thereof. In an aspect, quality of life can be measured objectivity and/or subjectively.

In an aspect, when administered, a disclosed pharmaceutical formulation comprising one or more disclosed HDAC11-activated M2 macrophages or a disclosed pharmaceutical formulation comprising one or more disclosed composition comprising HDAC11-activated M2 macrophages can prevent the rejection of one or more transplanted organs in the subject, can minimize the risk of rejection of one or more transplanted organs in the subject, can improve and/or enhance the healing of one or more chronic wounds in the subject, can improve and/or enhance healing of one or more chronic wounds, can improve and/or enhance angiogenesis, innervation, cellular migration, or any combination thereof in and/or around one or more chronic wounds in the subject, can improve and/or enhance cellular regeneration and/or cellular repair or one or more of the subject's affected body systems, can improve and/or enhance cellular regeneration and/or cellular repair in the subject's central nervous system and/or peripheral nervous system, can improve and/or enhance cellular regeneration and/or cellular repair in the subject's integumentary system, can improve and/or enhance cellular regeneration and/or cellular repair in the subject's digestive system, or any combination thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be formulated with one or more therapeutic agents and/or active agents. In an aspect, disclosed therapeutic agents and/or active agents can comprise (i) one or more biologically active agents, (ii) one or more pharmaceutically active agents, (iii) one or more immune-based therapeutic agents, (iv) one or more clinically approved agents, (v) one or more proteasome inhibitors, (vi) one or more immunosuppressives or immunosuppressive agents, or (vii) any combination thereof. In an aspect, an immunosuppressive agent can be anti-thymocyte globulin (ATG), cyclosporine (CSP), mycophenolate mofetil (MMF), or a combination thereof. In an aspect, a disclosed pharmaceutical formulation comprising one or more disclosed HDAC11-activated M2 macrophages or a disclosed pharmaceutical formulation comprising one or more disclosed composition comprising HDAC11-activated M2 macrophages can be formulated for systemic administration or for local administration.

### 3. Kits

Disclosed herein is a kit comprising one or more disclosed macrophages, one or more disclosed pharmaceutical formulations, one or more disclosed compositions, one or more disclosed therapeutic agents and/or disclosed active agents, or any combination thereof. Disclosed herein is a kit comprising HDAC11-activated macrophages or a composition thereof. Disclosed herein is a kit comprising HDAC11-activated M2 macrophages or a composition thereof. Disclosed herein is a kit comprising HDAC11-activated macrophages or a composition thereof made by a method disclosed herein. Disclosed herein is a kit comprising HDAC11-activated M2 macrophages or a composition thereof made by a method disclosed herein. Disclosed herein is a kit comprising HDAC11-activated macrophages or a composition thereof made by a method comprising contacting a population of naïve macrophages with one or more HDAC11 inhibitors. Disclosed herein is a kit comprising HDAC11-activated macrophages or a composition thereof made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein is a kit comprising HDAC11-activated macrophages or a composition thereof made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein is a kit comprising HDAC11-activated macrophages or a composition thereof made by a method comprising contacting a population of polarized macrophages with one or more HDAC11 inhibitors. Disclosed herein is a kit comprising HDAC11-activated macrophages or a composition thereof made by a method comprising contacting a population of polarized macrophages with one or more HDAC11 inhibitors, wherein the macrophages are polarized towards an anti-inflammatory phenotype. Disclosed herein is a kit comprising HDAC11-activated macrophages or a composition thereof made by a method comprising contacting a population of polarized macrophages with one or more HDAC11 inhibitors, wherein the macrophages are polarized towards a M2 phenotype. Disclosed herein is a kit comprising cryopreserved HDAC11-activated macrophages. Disclosed herein is a kit comprising cryopreserved HDAC11-activated M2 macrophages. Disclosed herein is a kit comprising cryopreserved HDAC11-activated macrophages made by a method disclosed herein. Disclosed herein is a kit comprising cryopreserved HDAC11-activated M2 macrophages made by a method disclosed herein. Disclosed herein is a kit comprising cryopreserved HDAC11-activated macrophages made by a method comprising contacting a population of naïve macrophages with one or more HDAC11 inhibitors. Disclosed herein is a kit comprising cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein is a kit comprising cryopreserved HDAC11-activated macrophages made by a method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein is a kit comprising cryopreserved HDAC11-activated macrophages made by a method comprising contacting a population of polarized macrophages with one or more HDAC11 inhibitors. Disclosed herein is a kit comprising cryopreserved HDAC11-activated macrophages made by a method comprising contacting a population of polarized macrophages with one or more HDAC11 inhibitors, wherein the macrophages are polarized towards an anti-inflammatory phenotype. Disclosed herein is a kit comprising cryopreserved HDAC11-activated macrophages made by a method comprising contacting a population of polarized macrophages with one or more HDAC11 inhibitors, wherein the macrophages are polarized towards a M2 phenotype.

Disclosed herein is a kit comprising a pharmaceutical formulation comprising one or more disclosed HDAC11-activated M2 macrophages, and one or more pharmaceutically acceptable carrier. Disclosed herein is a kit comprising a pharmaceutical formulation comprising one or more disclosed composition comprising HDAC11-activated macrophages, and one or more pharmaceutically acceptable carrier. Disclosed herein is a kit comprising a pharmaceutical formulation comprising one or more disclosed cryopreserved HDAC11-activated M2 macrophages, and one or more pharmaceutically acceptable carrier. Disclosed herein is a kit comprising a pharmaceutical formulation comprising one or more disclosed composition comprising cryopreserved HDAC11-activated macrophages, and one or more pharmaceutically acceptable carrier.

In an aspect, a disclosed kit can comprise at least two components constituting the kit. Together, the components constitute a functional unit for a given purpose (such as, for example, performing any aspect of a disclosed method including preparing the components used in a disclosed method). Individual member components can be physically packaged together or separately. For example, a kit comprising an instruction for using the kit can or cannot physically include the instruction with other individual member components. Instead, the instruction can be supplied as a separate member component, cither in a paper form or an electronic form which can be supplied on computer readable memory device or downloaded from an internet website, or as recorded presentation. In an aspect, a kit for use in a disclosed method can comprise one or more containers holding a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container can be an intravenous solution bag or a vial having a stopper pierceable by a hypodermic injection needle). In an aspect, a disclosed kit can comprise a “package insert”. In an aspect, a package insert can refer to instructions customarily included in commercial packages of therapeutic products, that contain information about the indications, usage, dosage, administration, contraindications and/or warnings concerning the use of such therapeutic products. The label or package insert can indicate that a disclosed population of macrophages, a disclosed population of M2 macrophages, a disclosed population of HDAC11-activated M2 macrophages, or a disclosed composition comprising a disclosed population of macrophages, a disclosed population of M2 macrophages, or a disclosed population of HDAC11-activated M2 macrophages, or a disclosed pharmaceutical formulation comprising a disclosed composition or a disclosed pharmaceutical formulation comprising a disclosed population of macrophages, a disclosed therapeutic agent, a disclosed active agent, or any combination thereof. A kit can comprise additional components necessary for administration such as, for example, other buffers, diluents, filters, needles, and syringes.

In an aspect, a disclosed kit can be used to decrease and/or diminish inflammation in one or more of the subject's body systems, can diminish and/or decrease the pathology of the subject's disease or disorder, can diminish and/or decrease the subject's symptoms, can improve and/or enhance the subject's quality of life, can spare the rejection of one or more transplanted organs can be spared from rejection, can improve and/or enhance the healing of the subject's one or more chronic wounds, can improve and/or enhance one or more chronic wounds, can improve angiogenesis, innervation, cellular migration, or any combination thereof in a subject, can improve and/or elicit signs of cellular regeneration and/or cellular repair (e.g., in the cardiovascular system, in the central nervous system and/or peripheral nervous system, the integumentary system, the digestive system, or any combination thereof), or any combination thereof.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, about 50 nM to about 25 μM of a disclosed HDAC11 inhibitor can polarize resting or M0 macrophages into a M2 phenotype or an anti-inflammatory phenotype.

### J. Methods using Disclosed Compositions

### 1. Methods of Producing HDAC11-Activated Macrophages

Disclosed herein is a method of producing HDAC11-activated macrophages, the method comprising contacting a population of naïve macrophages with one or more HDAC11 inhibitors. Disclosed herein is method of producing HDAC11-activated macrophages, the method comprising contacting a population of naïve macrophages with one or more HDAC11 inhibitors, wherein following the contacting of the naïve macrophages with HDAC11 inhibitor, the HDAC11-activated macrophages are then polarized towards an anti-inflammatory phenotype. Disclosed herein is a method of producing HDAC11-activated macrophages, the method comprising contacting a population of naïve macrophages with one or more HDAC11 inhibitors, wherein following the contacting of the naïve macrophages with HDAC11 inhibitor, the HDAC11-activated macrophages are then polarized towards a M2 phenotype. Disclosed herein is a method of producing HDAC11-activated macrophages, the method comprising contacting a population of polarized macrophages with one or more HDAC11 inhibitors. Disclosed herein is a method of producing HDAC11-activated macrophages, the method comprising contacting a population of polarized macrophages with one or more HDAC11 inhibitors, wherein the macrophages are polarized towards an anti-inflammatory phenotype. Disclosed herein is a method of producing HDAC11-activated macrophages, the method comprising contacting a population of polarized macrophages with one or more HDAC11 inhibitors, wherein the macrophages are polarized towards a M2 phenotype.

In an aspect of a disclosed method, contacting a population of M0 or naïve macrophages with one or more HDAC11 inhibitors can enhance and/or improve the M2 phenotype of the macrophages upon polarization. In an aspect of a disclosed method, contacting a population of polarized macrophages with one or more HDAC11 inhibitors can enhance and/or improve the M2 phenotype of the polarized macrophages. In an aspect of a disclosed method, contacting a population of M0 or naïve macrophages with one or more HDAC11 inhibitors can enhance and/or improve the anti-inflammatory phenotype of the macrophages upon polarization. In an aspect of a disclosed method, contacting a population of polarized macrophages with one or more HDAC11 inhibitors can enhance and/or improve the anti-inflammatory phenotype of the polarized macrophages.

In an aspect, HDAC11-activated macrophages or HDAC11 polarized macrophage can refer to a M0 or naïve macrophage that has been treated ex vivo with an HDAC11 inhibitor (such as, for example a disclosed HDAC11 inhibitor or disclosed selective HDAC11 inhibitor).

In an aspect, disclosed HDAC11-activated macrophages are first treated ex vivo with a disclosed HDAC11 inhibitor and then treated ex vivo with a disclosed macrophage polarizing agent. In an aspect, disclosed HDAC11 polarized macrophages are first treated ex vivo with a macrophage polarizing agent and then treated ex vivo with a disclosed HDAC11 inhibitor.

In an aspect of a disclosed method, wherein contacting a population of naïve macrophages with an HDAC11 inhibitor comprises a pre-determined amount of time. In an aspect of a disclosed method, contacting a population of naïve macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of naïve macrophages with an HDAC11 inhibitor can comprise more than 24 hours.

In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect of a disclosed method, contacting a population of polarized macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of polarized macrophages with an HDAC11 inhibitor can comprise more than 24 hours. In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, naïve macrophages can be contacted with an HDAC inhibitor one or more times, and then polarized to M2 macrophages. In an aspect, polarized macrophages can be contacted with an HDAC inhibitor one or more times. In an aspect of a disclosed method of producing HDAC11-activated macrophages, a disclosed subject can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, a disclosed subject can have one or more foot ulcers. In an aspect, a disclosed subject can have diabetes. In an aspect, a disclosed subject can be the recipient of one or more solid organ transplants. In an aspect, a disclosed subject can have irritable bowel disease (IBD). In an aspect, a disclosed subject can have one or more inflammatory diseases. In an aspect, a disclosed subject can have one or more skin inflammatory diseases. In an aspect, a disclosed subject can have suffered one or more burns. In an aspect, a disclosed subject can have incurred an ischemic injury. In an aspect, a disclosed ischemic injury can affect the subject's brain and/or the subject's heart. In an aspect, a disclosed subject can have suffered one or more myocardial infarctions. In an aspect, a disclosed subject can have suffered one or more transient ischemic attacks. In an aspect, a disclosed subject can have an autoinflammatory disease (AIF). In an aspect, a disclosed autoinflammatory disease can comprise familial Mediterranean fever (FMF), hyper IgD syndrome (HIDS), tumor necrosis factor receptor associated autoinflammatory syndrome (TRAPS), cryopyrin associated periodic syndromes (CAPS), or Blau syndrome and pyogenic sterile arthritis pyoderma gangrenosum and acne syndrome (PAPA).

In an aspect, a disclosed subject can have an autoimmune disease (AD). In an aspect, a disclosed autoimmune disease or disorder can comprise *Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, *Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), *Autoimmune hyperlipidemia, *Autoimmune hypophysitis, *Autoimmune inner ear disease (AIED), *Autoimmune lymphoproliferative syndrome (ALPS), *Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, *Autoimmune retinopathy (AIR), *Autoimmune sudden sensorineural hearing loss (SNHL), Balo disease, Behçet's disease, *Birdshot chorioretinopathy/birdshot uveitis, Bullous pemphigoid, *Castleman disease, Celiac disease, *Chagas disease, Chronic inflammatory demyelinating polyneuropathy (CIDP), *Chronic urticaria (CU), Churg-Strauss syndrome/cosinophilic granulomatosis with polyangiitis (EGPA), Cogan's syndrome, Cold agglutinin disease, CREST syndrome|limited cutaneous systemic sclerosis, Crohn's disease (CD), *Cronkhite-Canada syndrome (CSS), *Cryptogenic organizing pneumonia (COP), Dermatitis herpetiformis, Dermatomyositis, Devic's disease/neuromyelitis optica (NMO), Diabetes, type 1, Discoid lupus, *Dressler's syndrome/postmyocardial infarction/postpericardiotomy syndrome, *Eczema/Atopic Dermatitis, *Endometriosis, *Eosinophilic esophagitis, Eosinophilic fasciitis, *Erythema nodosum, *Essential mixed cryoglobulinemia, Evans syndrome, *Fibrosing alveolitis/Idiopathic pulmonary fibrosis (IPF), *Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, *Hidradenitis suppurativa, Hurst's disease/acute hemorrhagic leukoencephalitis (AHLE), *Hypogammaglobulinemia, IgA nephropathy/Berger's disease, Immune-mediated necrotizing myopathy (IMNM), Immune thrombocytopenia (ITP)/autoimmune thrombocytopenia purpura/autoimmune thrombocytopenia, *Inclusion body myositis, *IgG4-related sclerosing disease (ISD), *Interstitial cystitis, Juvenile idiopathic arthritis/Adult-onset Still's disease, Juvenile polymyositis|Juvenile dermatomyositis|juvenile myositis, *Kawasaki disease, Lambert-Eaton myasthenic syndrome (LEMS), *Leukocytoclastic vasculitis, *Lichen planus, *Lichen sclerosus, *Ligncous conjunctivitis, Linear IgA disease (LAD)|linear IgA bullous dermatosis (LABD), Lupus nephritis, *Lyme disease/chronic Lyme disease/post-treatment Lyme disease syndrome (PTLDS), *Lymphocytic colitis/microscopic colitis, Ménière's disease, *Microscopic polyangiitis (MPA)/ANCA-associated vasculitis, Mixed connective tissue disease (MCTD), *Mooren's ulcer, *Mucha-Habermann disease, *Multifocal motor neuropathy, Multiple sclerosis (MS), *Myalgic encephalomyelitis (ME)/Chronic fatigue syndrome (CFS), Myasthenia gravis (MG), *Narcolepsy, Ocular cicatricial pemphigoid, *Opsoclonus-myoclonus syndrome (OMS), Palindromic rheumatism, Paraneoplastic cerebellar degeneration, Parancoplastic pemphigus, *Parry-Romberg syndrome (PRS)/Hemifacial atrophy (HFA)/Progressive facial hemiatrophy, Paroxysmal nocturnal hemoglobinuria (PNH), *Peripheral uveitis/pars planitis, *PANS/PANDAS, Parsonage-Turner syndrome, Pemphigus gestationis/herpes gestationis, Pomphigus foliaceus, Pemphigus vulgaris, *Pernicious anemia, POEMS syndrome, Polyarteritis nodosa, Polymyalgia rheumatica, Polymyositis, *Postural orthostatic tachycardia syndrome (POTS), Primary biliary cirrhosis (PBC)/primary biliary cholangitis, *Primary sclerosing cholangitis (PSC), Psoriasis, Palmoplantar Pustulosis, Psoriatic arthritis, *Pulmonary fibrosis, idiopathic (IPF), Pure red cell aplasia (PRCA), *Pyoderma gangrenosum, Raynaud's syndromc/phenomenon, Reactive arthritis/Reiter's syndrome, *Reflex sympathetic dystrophy syndrome (RSD)/Complex regional pain syndrome (CRPS), Relapsing polychondritis, *Restless leg syndrome (RLS)/Willis-Ekbom disease, Rheumatic fever, Rheumatoid arthritis, Sarcoidosis, Schmidt syndrome/autoimmune polyendocrine syndrome type II, *Scleritis, Scleroderma, *Serpiginous choroidopathy, Sjögren's syndrome, *Stiff person syndrome (SPS), Small fiber sensory neuropathy, Systemic lupus erythematosus (SLE), *Subacute bacterial endocarditis (SBE), *Susac syndrome, *Sydenham's chorea, *Sympathetic ophthalmia, *Takayasu's arteritis (vasculitis), Testicular autoimmunity (vasculitis, orchitis), *Tolosa-Hunt syndrome, *Transverse myelitis (TM), *Tubulointerstitial nephritis uveitis syndrome (TINU), Ulcerative colitis (UC), Undifferentiated connective tissue disease (UCTD), *Uveitis|anterior/intermediate/posterior, *Vasculitis, Vitiligo, *Vogt-Koyanagi-Harada syndrome (VKH), or any combination thereof.

In an aspect, a subject can be male or female. In an aspect, a subject can be an adult, a teenager, an adolescent, a child, a toddler, a baby, or an infant. In an aspect, a subject can have received treatment for one or more disclosed AIFs or ADs. In an aspect, a subject can be treatment-naïve.

In an aspect, a disclosed method of producing HDAC11-activated macrophages can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample can be collected from a subject prior to treatment, during treatment, after treatment, or any combination thereof. In an aspect, a blood sample can be collected from a subject at any time deemed medically and/or clinically appropriate by the skilled clinician. In an aspect, a disclosed method of producing HDAC11-activated macrophages can further comprise isolating monocytes from peripheral blood monocular cells in the subject's blood sample. In an aspect, a disclosed method of producing HDAC11-activated macrophages can further comprise isolating bone marrow derived monocytes from the subject's blood sample. In an aspect, a disclosed method of producing HDAC11-activated macrophages can further comprise isolating monocytes from the subject's blood sample. In an aspect, a disclosed method of producing HDAC11-activated macrophages can further comprise isolating naïve macrophages (M0) from the subject's blood sample. In an aspect, a disclosed method of producing HDAC11-activated macrophages can further comprise subjecting a disclosed blood sample to centrifugation. In an aspect, a disclosed method can further comprise separating the blood sample into its component parts using, for example, centrifugation, apheresis, or any technique to the skilled person. In an aspect, a disclosed separating step can comprise generating a layer of clear fluid, a layer of red fluid, and a thin layer in between the clear fluid layer and the red fluid layer. In an aspect, a disclosed red layer can comprise red blood cells. In an aspect, a disclosed clear layer can comprise plasma. In an aspect, a disclosed thin layer in between the red layer and the clear layer can comprise the buffy coat. In an aspect, a disclosed buffy coat can comprise white blood cells and platelets. In an aspect, a disclosed method can further comprise isolating peripheral blood mononuclear cells (PMBCs) from the buffy coat. In an aspect, PMBCs can comprise lymphocytes and/or monocytes. In an aspect, macrophages can be derived from monocytes. In an aspect, isolating monocytes can be done by any method and/or technique known to the skilled person. For example, in an aspect, a conical blood filter and the buffy coat having approximately 10 mL and 70 mL respectively, can be used. In an aspect, a disclosed sample can be collected in a tube, e.g., a 50 mL tube. In an aspect, if using a conical blood filter, then dilute the sample by adding PBS, for example, at room temperature. In an aspect, a disclosed method can add 10 mL of Ficoll-Hypaque-1077 (or Lymphoprep) to a disclosed centrifuge tube at room temperature. In an aspect, a disclosed method can comprise carefully layering the blood (e.g., 3 mL) onto the Ficoll-Hypaque-1077. In an aspect, blood can stay in the upper layer and the Ficoll-Hypaque 1077 in the lowest one. In an aspect, a disclosed method can comprise centrifuging at 400×g for about 30 min at room temperature. In an aspect of a disclosed method, the centrifuge can be set for 0 (zero) acceleration and 0 (zero) break. In an aspect, after centrifugation, 3 layers can be visible. In an aspect, an upper layer can comprise plasma and the Ficoll-Hypaque. In an aspect, an opaque interface can comprise the mononuclear cells. In an aspect, a lower lever can comprise the red blood cells. In an aspect of a disclosed method, a Pasteur pipette can be used to aspirate the upper layer until close to the opaque phase comprising the mononuclear cells. In an aspect, the upper layer can be discarded. In an aspect of a disclosed method, the disclosed opaque interface can be transferred to a tube (e.g., 50 mL). In an aspect, a tube can be filled with cold PBS. In an aspect of a disclosed method can comprise centrifuged at 250×g for about 10 min at 4° C. In an aspect, a disclosed centrifuge can be at full acceleration and break 5. In an aspect of the disclosed method, at the end of the first washing, the supernatant can appear turbid due to presence of platelets. In an aspect of a disclosed method, the supernatant can be aspirated and the pellet can be resuspended with cold PBS. In an aspect of a disclosed method, centrifuging can be at 250×g for about 10 min at 4° C. In an aspect of a disclosed method, washing steps can be repeated 3-4 times or can be repeated until a clear supernatant is obtained. In an aspect of a disclosed method, monocytes can be isolated by negative selection and/or cell adhesion. Both of these techniques are known to the skilled person in the art. In an aspect, a disclosed method of producing HDAC11-activated macrophages can further comprise isolating resting or M0 macrophages from the buffy coat. In an aspect, a disclosed method of producing HDAC11-activated macrophages can further comprise differentiating monocytes into resting or M0 macrophages. In an aspect of a disclosed method of producing HDAC11-activated macrophages, disclosed macrophages can be resting or M0 macrophages. In an aspect, a disclosed method of producing HDAC11-activated macrophages can further comprise polarizing the resting or M0 macrophages into a M2 phenotype or anti-inflammatory phenotype. In an aspect, a disclosed method of producing HDAC11-activated macrophages can further comprise polarizing the resting or M0 macrophages into an alternatively activated macrophage phenotype. In an aspect, a disclosed method of producing HDAC11-activated macrophages can further comprise polarizing the resting or M0 macrophages into a M2 phenotype or anti-inflammatory phenotype comprises contacting the resting or M0 macrophages with IL-4, IL-10, IL-13, TGFβ-1, PGE2, or any combination thereof. In an aspect of a disclosed method of producing HDAC11-activated macrophages, resting or M0 macrophages can be polarized into a M2 phenotype or anti-inflammatory phenotype. In an aspect of a disclosed method of producing HDAC11-activated macrophages, resting or M0 macrophages can be polarized into an alternatively activated macrophage phenotype. In an aspect of a disclosed method of producing HDAC11-activated macrophages, resting or M0 macrophages can be polarized into a M2 phenotype or anti-inflammatory phenotype comprises contacting the resting or M0 macrophages with IL-4, IL-10, IL-13, TGFβ-1, PGE2, or any combination thereof. In an aspect of a disclosed method of producing HDAC11-activated macrophages, polarizing resting or M0 macrophages into a M2 phenotype or an anti-inflammatory phenotype can comprise about a 1-day to about a 10-day incubation. In an aspect of a disclosed method of producing HDAC11-activated macrophages, polarizing resting or M0 macrophages into a M2 phenotype or an anti-inflammatory phenotype can comprise about a 3-day to about a 7-day incubation. In an aspect of a disclosed method of producing HDAC11-activated macrophages, polarizing the resting or M0 macrophages into a M2 phenotype or an anti-inflammatory phenotype can comprise about a 1-day incubation, about a 2-day incubation, about a 3-day incubation, about a 4-day incubation, about a 5-day incubation, about a 6-day incubation, about a 7-day incubation, about a 8-day incubation, about a 9-day incubation, about a 10-day incubation, or more than a 10-day incubation. In an aspect of a disclosed method of producing HDAC11-activated macrophages, wherein following the contacting of the resting or M0 macrophages with a disclosed HDAC11 inhibitor, polarizing macrophages can be incubated from about 1 day to about 10 days.

In an aspect of a disclosed method of producing HDAC11-activated macrophages, wherein following the contacting of the resting or M0 macrophages with a disclosed HDAC11 inhibitor, polarizing macrophages can be incubated from about 3 days to about 7 days. In an aspect of a disclosed method of producing HDAC11-activated macrophages, wherein following the contacting of the resting or M0 macrophages with a disclosed HDAC11 inhibitor, polarizing macrophages can be incubated for about 1 day, about 2 days, about 3 days, about 4 days, about 5 days, about 6 days, about 7 days, about 8 days, about 9 days, about 10 days, or more than 10 days.

In an aspect, one or more steps of a disclosed method of producing HDAC11-activated macrophages can be performed ex vivo.

In an aspect of a disclosed method of producing HDAC11-activated macrophages, a disclosed HDAC11 inhibitor can comprise elevenostat (JB3-22), garcinol, SIS7, SIS17, FT895, romidepsin, quisinostat, or any combination thereof. In an aspect of a disclosed method of producing HDAC11-activated macrophages, a disclosed HDAC11 inhibitors can comprise any commercially available HDAC11 inhibitor or a combination of commercially available HDAC11 inhibitors. In an aspect, an HDAC11 inhibitor can comprise a selective HDAC11 inhibitor or HDAC11 selective inhibitor and can refer to a compound that preferentially inhibits histone deacetylase 11 over one or more other histone deacetylase isoforms, e.g., HDAC1, HDAC2, HDAC3, HDAC4, HDAC5, HDAC6, HDAC7, HDAC8, HDAC9, and/or HDAC10 in a cell-based in vitro assay. For example, a compound having a HDAC11 IC_{SO}=5 nM and a HDAC1 IC_{SO} of 500 nM is a selective HDAC11 inhibitor that is 100-fold more selective over HDAC1; a compound having a HDAC11 IC_{SO}=5 nM, a HDAC1 IC_{SO}=500 nM, and a HDAC3 IC_{SO}=50 nM is a selective HDAC11 inhibitor that is 100-fold more selective over HDAC1 and 10-fold more selective over HDAC3; and so on. In one aspect, a selective HDAC11 inhibitor can preferentially inhibit HDAC11 over HDAC1, HDAC2, HDAC3, HDAC4, HDAC5, HDAC6, HDAC7, HDAC8, HDAC9, and/or HDAC10. In an aspect, the selective HDAC11 inhibitor can preferentially inhibit HDAC11 over one or more other HDAC isoforms. In an aspect, the selective HDAC11 inhibitor can be at least about 5-fold more selective over one or more other HDAC isoforms. In an aspect, the selective HDAC11 inhibitor can be at least about 10-fold more selective over one or more other HDAC isoforms. In an aspect, the selective HDAC11 inhibitor can be at least about 15-fold more selective over one or more other HDAC isoforms. In aspect, the selective HDAC11 inhibitor can be at least about 20-fold more selective over one or more other HDAC isoforms. In an aspect, the selective HDAC11 inhibitor can be at least about 30-fold more selective over one or more other HDAC isoforms. In an aspect, the selective HDAC11 inhibitor can be at least about 40-fold more selective over one or more other HDAC isoforms. In an aspect, the selective HDAC11 inhibitor can be at least about 50-fold more selective over one or more other HDAC isoforms. In an aspect, the selective HDAC6 inhibitor can be at least about 100-fold more selective over one or more other HDAC isoforms. In another aspect, the selective HDAC11 inhibitor can be at least about 150-fold more selective over one or more other HDAC isoforms. In an aspect, the selective HDAC11 inhibitor can be at least about 200-fold more selective over one or more other HDAC isoforms. In an aspect, the selective HDAC11 inhibitor can be at least about 250-fold more selective over one or more other HDAC isoforms. In an aspect, the selective HDAC11 inhibitor can be at least about 500-fold more selective over one or more other HDAC isoforms. In an aspect, the selective HDAC11 inhibitor can be at least about 750-fold more selective over one or more other HDAC isoforms. In an aspect, the selective HDAC11 inhibitor can be at least about 1000-fold more selective over one or more other HDAC isoforms. In an aspect, the selective HDAC11 inhibitor can be at least about 2000-fold more selective over one or more other HDAC isoforms. In an aspect, the selective HDAC11 inhibitor can be at least about 3000-fold more selective over one or more other HDAC isoforms. HDAC11 selectivity over the other HDAC isoforms in cell-based assays can be determined using methods known in the art. In another aspect, the selective HDAC11 inhibitor can be at about 10-fold to about 3000-fold more selective over one or more other HDAC isoforms. In an aspect, the selective HDAC11 inhibitor can be at about 20-fold to about 3000-fold more selective over one or more other HDAC isoforms. In an aspect, the selective HDAC11 inhibitor can be at about 50-fold to about 3000-fold more selective over one or more other HDAC isoforms. In an aspect, the selective HDAC11 inhibitor can be at about 100-fold to about 3000-fold more selective over one or more other HDAC isoforms. In an aspect, the selective HDAC11 inhibitor can be at about 500-fold to about 3000-fold more selective over one or more other HDAC isoforms.

In an aspect of a disclosed method, about 50 nM to about 25 μM of a disclosed HDAC11 inhibitor can polarize resting or M0 macrophages into a M2 phenotype or an anti-inflammatory phenotype.

In an aspect of a disclosed method of producing HDAC11-activated macrophages, contacting the resting or M0 macrophages with one or more HDAC11 inhibitors can occur prior to polarizing the resting or M0 macrophages into the M2 phenotype or anti-inflammatory phenotype. In an aspect of a disclosed method of producing HDAC11-activated macrophages, contacting the resting or M0 macrophages with one or more HDAC11 inhibitors can occur after polarizing the resting or M0 macrophages into the M2 phenotype or anti-inflammatory phenotype. In an aspect of a disclosed method of producing HDAC11-activated macrophages, contacting the resting or M0 macrophages with one or more HDAC11 inhibitors can occur during the polarizing the resting or M0 macrophages into the M2 phenotype or anti-inflammatory phenotype. In an aspect, a disclosed method of producing HDAC11-activated macrophages can further comprise confirming the resulting macrophages demonstrate the M2 phenotype or anti-inflammatory phenotype. In an aspect, confirming the M2 phenotype or anti-inflammatory phenotype can comprise detecting a change in the expression level of one or more relevant genes and/or relevant proteins. In an aspect of a disclosed method of producing HDAC11-activated macrophages, detecting a change in the expression level can comprise detecting an increase in the expression level of one or more relevant genes and/or relevant proteins. In an aspect, detecting a change in expression level can comprise detecting a decrease in the expression level of one or more relevant genes and/or relevant proteins. In an aspect of a disclosed method of producing HDAC11-activated macrophages, one or more disclosed relevant genes and/or relevant proteins can comprise IL-10, TGF-β, Fizz1, Arg1, Mrc1 (CD206), or any combination thereof. In an aspect, detecting a change in the expression level can comprise detecting an increase in the expression level of IL-10, TGF-β, Fizz1, Arg1, Mrc1 (CD206), or any combination thereof. In an aspect, a disclosed increase can comprise a 10%, 20%, 30%, 40%, 50%, 60%, 70%, 80%, 90%, 100%, or any amount of an increase when compared to a control subject (such as, for example, a subject that has not received HDAC11-activated macrophages). In an aspect, a disclosed increase can comprise a 10-20%, 20-30%, 30-40%, 40-50%, 50-60%, 60-70%, 70-80%, 80-90%, or 90-100% or any amount of an increase when compared to a control subject (such as a subject that has not received HDAC11-activated macrophages).

In an aspect, a disclosed decrease reduction can comprise a 10%, 20%, 30%, 40%, 50%, 60%, 70%, 80%, 90%, 100%, or any amount of a decrease when compared to a control subject (such as, for example, a subject that has not received HDAC11-activated macrophages)). In an aspect, a disclosed decrease can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated macrophages).

In an aspect of a disclosed method of producing HDAC11-activated macrophages, polarized M2 macrophages can be cryopreserved. In an aspect, cryopreserved polarized M2 macrophages can be thawed. In an aspect, thawing cryopreserved polarized M2 macrophages can be done by any technique known to the skilled person in the art. In an aspect, thawed polarized M2 macrophages can be administered to a subject. In an aspect of a disclosed method of producing HDAC11-activated macrophages, resting or M0 macrophages can be cryopreserved prior to polarization and/or activation. In an aspect, cryopreserved resting or M0 macrophages can be thawed and then can be polarized and/or activated. In an aspect, thawing cryopreserved resting or M0 macrophages can be done by any technique known to the skilled person in the art. In an aspect, thawed resting or M0 macrophages can be polarized and/or activated and can then be administered to a subject. In an aspect, a disclosed method of producing HDAC11-activated macrophages can further comprise administering to the subject the HDAC11-activated M2 macrophages or a composition comprising HDAC11-activated M2 macrophages.

In an aspect, administering can comprise intravenous administration of HDAC11-activated M2 macrophages or a composition comprising HDAC11-activated M2 macrophages. In an aspect, administering can comprise systemic administration or local administration of HDAC11-activated M2 macrophages or a composition comprising HDAC11-activated M2 macrophages. In an aspect, local administration can comprise delivery to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof. In an aspect, local delivery or local administration can refer to delivering or administering HDAC11-activated M2 macrophages or a composition comprising HDAC11-activated M2 macrophages directly to a target site within a subject. For example, HDAC11-activated M2 macrophages or a composition comprising HDAC11-activated M2 macrophages can be locally delivered by direct injection into an affected body system (such as one, for example, having inflammation). Local delivery or local administration can also include topical applications or localized injection techniques such as intramuscular, subcutaneous, and/or intradermal injection of HDAC11-activated M2 macrophages or a composition comprising HDAC11-activated M2 macrophages. In an aspect, local administration of HDAC11-activated M2 macrophages or a composition comprising HDAC11-activated M2 macrophages can comprise delivery to one or more body systems in need thereof. In an aspect, local administration or local delivery HDAC11-activated M2 macrophages or a composition comprising HDAC11-activated M2 macrophages can comprise a transdermal administration, administration via implant, administration via suppository, implantation via a biodegradable polymer or matrix, implantation via a biodegradable hydrogel, or any combination thereof. In an aspect of a disclosed method, administering HDAC11-activated M2 macrophages or a composition comprising HDAC11-activated M2 macrophages can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, HDAC11-activated M2 macrophages or a composition comprising HDAC11-activated M2 macrophages can be administered via multiple routes of administration. In an aspect of a disclosed method of producing HDAC11-activated macrophages, following administration of HDAC11-activated M2 macrophages or a composition comprising HDAC11-activated M2 macrophages to the subject, inflammation in one or more of the subject's body systems can be diminished and/or decreased. In an aspect of a disclosed method of producing HDAC11-activated macrophages, the decrease in inflammation in one or more of the subject's body systems can be measured objectively and/or subjectively.

In an aspect of a disclosed method of producing HDAC11-activated macrophages, following administration of HDAC11-activated M2 macrophages or a composition comprising HDAC11-activated M2 macrophages to the subject, the pathology of the subject's disease or disorder can be diminished and/or decreased. In an aspect of a disclosed method of producing HDAC11-activated macrophages, wherein, following administration of HDAC11-activated M2 macrophages or a composition comprising HDAC11-activated M2 macrophages to the subject, the subject's symptoms can be diminished and/or decreased. In an aspect of a disclosed method of producing HDAC11-activated macrophages, wherein, following administration of HDAC11-activated M2 macrophages or a composition comprising HDAC11-activated M2 macrophages to the subject, the subject's quality of life can be improved and/or enhanced. In an aspect of a disclosed method of producing HDAC11-activated macrophages, wherein quality of life can be measured objectivity and/or subjectively.

In an aspect of a disclosed method of producing HDAC11-activated macrophages, wherein, following administration of HDAC11-activated M2 macrophages or a composition comprising HDAC11-activated M2 macrophages to the subject, the one or more transplanted organs can be not rejected. In an aspect of a disclosed method of producing HDAC11-activated macrophages, wherein, following administration of HDAC11-activated M2 macrophages or a composition comprising HDAC11-activated M2 macrophages to the subject, the one or more transplanted organs can be spared from rejection.

In an aspect of a disclosed method of producing HDAC11-activated macrophages, wherein, following administration of HDAC11-activated M2 macrophages or a composition comprising HDAC11-activated M2 macrophages to the subject, the healing of the subject's one or more chronic wounds can be improved and/or enhanced. In an aspect of a disclosed method of producing HDAC11-activated macrophages, wherein improving and/or enhancing healing of one or more chronic wounds can comprise improving angiogenesis, innervation, cellular migration, or any combination thereof. In an aspect of a disclosed method of producing HDAC11-activated macrophages, wherein following administration of HDAC11-activated M2 macrophages or a composition comprising HDAC11-activated M2 macrophages to the subject, one or more of the subject's body systems can experience and/or show signs of cellular regeneration and/or cellular repair. In an aspect of a disclosed method of producing HDAC11-activated macrophages, wherein the one or more body systems experiencing and/or showing signs of regeneration and/or repair can comprise the cardiovascular system.

In an aspect of a disclosed method of producing HDAC11-activated macrophages, wherein previously ischemic and/or infarcted and/or languishing areas of the cardiovascular system can show signs of regeneration and/or repair. In an aspect of a disclosed method of producing HDAC11-activated macrophages, wherein the one or more body systems experiencing and/or showing signs of regeneration and/or repair can comprise the central nervous system and/or peripheral nervous system. In an aspect of a disclosed method of producing HDAC11-activated macrophages, wherein previously ischemic and/or infarcted/or languishing areas of the central nervous system and/or peripheral nervous system can show signs of regeneration and/or repair. In an aspect of a disclosed method of producing HDAC11-activated macrophages, wherein the one or more body systems experiencing and/or showing signs of regeneration and/or repair can comprise the integumentary system.

In an aspect of a disclosed method of producing HDAC11-activated macrophages, wherein the one or more body systems experiencing and/or showing signs of regeneration and/or repair can comprise the digestive system. In an aspect, a disclosed method of producing HDAC11-activated macrophages can further comprise diminishing and/or decreasing inflammation in one or more of the subject's body systems. Body systems are disclosed supra.

In an aspect, a decrease in inflammation in one or more of the subject's body systems can be measured objectively and/or subjectively. In an aspect, a disclosed method of producing HDAC11-activated macrophages can further comprise diminishing and/or decreasing the pathology of the subject's disease or disorder. In an aspect, a disclosed method of producing HDAC11-activated macrophages can further comprise diminishing and/or decreasing the subject's symptoms. In an aspect, a disclosed method of producing HDAC11-activated macrophages can further comprise improving and/or enhancing the subject's quality of life. In an aspect, quality of life can be measured objectivity and/or subjectively.

In an aspect, a disclosed method of producing HDAC11-activated macrophages can further comprise preventing the rejection of one or more transplanted organs in the subject. In an aspect, a disclosed method of producing HDAC11-activated macrophages can further comprise minimizing the risk of rejection of one or more transplanted organs in the subject. In an aspect, a disclosed method of producing HDAC11-activated macrophages can further comprise improving and/or enhancing the healing of one or more chronic wounds in the subject. In an aspect, a disclosed method of producing HDAC11-activated macrophages can further comprise and/or enhancing healing of one or more chronic wounds. In an aspect, a disclosed method of producing HDAC11-activated macrophages can further comprise improving and/or enhancing angiogenesis, innervation, cellular migration, or any combination thereof in and/or around one or more chronic wounds in the subject.

In an aspect, a disclosed method of producing HDAC11-activated macrophages can further comprise improving and/or enhancing cellular regeneration and/or cellular repair or one or more of the subject's affected body systems. In an aspect, a disclosed method of producing HDAC11-activated macrophages can further comprise improving and/or enhancing cellular regeneration and/or cellular repair in the subject's central nervous system and/or peripheral nervous system. In an aspect, a disclosed method of producing HDAC11-activated macrophages can further comprise improving and/or enhancing cellular regeneration and/or cellular repair in the subject's integumentary system. In an aspect, a disclosed method of producing HDAC11-activated macrophages can further comprise improving and/or enhancing cellular regeneration and/or cellular repair in the subject's digestive system.

In an aspect, a disclosed method of producing HDAC11-activated macrophages can further comprise repeating one or more times one or more steps of a disclosed method. In an aspect, a disclosed obtaining step can be repeated. In an aspect, a disclosed isolating step can be repeated. In an aspect, a disclosed contacting step can be repeated. In an aspect, a disclosed activating step can be repeated. In an aspect, a disclosed polarizing step can be repeated. In an aspect, a disclosed incubating step can be repeated. In an aspect, a disclosed administering step can be repeated. In an aspect, a disclosed method can comprise repeating an obtaining step, repeating an isolating step, repeating a contacting step, repeating an activating step, repeating a polarizing step, repeating an incubating step, repeating an administering step, or repeating one or more time any combination of steps thereof. In an aspect, a disclosed improvement and/or enhancement (such as, for example, in the subject's quality of life and/or movement and/or would healing and/or angiogenesis, etc.) can comprise a 10%, 20%, 30%, 40%, 50%, 60%, 70%, 80%, 90%, 100%, or any amount of improvement and/or enhancement when compared to a control subject (such as, for example, a subject that has not received HDAC11-activated M2 macrophages or a composition comprising HDAC11-activated M2 macrophages). In an aspect, a disclosed improvement and/or enhancement (such as, for example, in the subject's quality of life and/or movement and/or would healing and/or angiogenesis, etc.) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not HDAC11-activated M2 macrophages or a composition comprising HDAC11-activated M2 macrophages).

In an aspect, a disclosed diminishment (such as, for example, in the subject's pain and/or wound size and/or restrictions) can comprise a 10%, 20%, 30%, 40%, 50%, 60%, 70%, 80%, 90%, 100%, or any amount of a decrease when compared to a control subject (such as, for example, a subject that has not received HDAC11-activated M2 macrophages or a composition comprising HDAC11-activated M2 macrophages). In an aspect, a disclosed diminishment (such as, for example, in the subject's pain and/or wound size and/or restrictions) can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated M2 macrophages or a composition comprising HDAC11-activated M2 macrophages). In an aspect, a disclosed method can further comprise administering to a disclosed subject one or more therapeutic agents and/or active agents.

In an aspect, one or more disclosed therapeutic agents and/or active agents can comprise an immune modulator. In an aspect, a disclosed immune modulator can refer to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions.

In an aspect, administering can comprise intravenous administration of one or more therapeutic agents and/or active agents. In an aspect, administering can comprise systemic administration or local administration of one or more therapeutic agents and/or active agents. In an aspect, local delivery or local administration can refer to delivering or administering one or more therapeutic agents and/or active agents directly to a target site within a subject. For example, the one or more therapeutic agents and/or active agents can be locally delivered by direct injection into an affected body system (such as one, for example, having inflammation). Local delivery or local administration can also include topical applications or localized injection techniques such as intramuscular, subcutaneous, and/or intradermal injection of one or more therapeutic agents and/or active agents. In an aspect, local administration of one or more therapeutic agents and/or active agents can comprise delivery to one or more body systems in need thereof. In an aspect, local administration or local delivery of one or more therapeutic agents and/or active agents can comprise a transdermal administration, administration via implant, administration via suppository, implantation via a biodegradable polymer or matrix, implantation via a biodegradable hydrogel, or any combination thereof. A skilled clinician can determine the best route of administration for a subject at a given time.

In an aspect of a disclosed method of producing HDAC11-activated macrophages, HDAC11-activated M2 macrophages or a composition comprising HDAC11-activated M2 macrophages can be administered to the subject prior to, concurrent with, or after administration of one or more therapeutic agents and/or active agents. In an aspect of a disclosed method, HDAC11-activated M2 macrophages or a composition comprising HDAC11-activated M2 macrophages can be administered one or more times and the one or more therapeutic agents and/or active agents can be administered one or more times. In an aspect of a disclosed method, HDAC11-activated M2 macrophages or a composition comprising HDAC11-activated M2 macrophages can be administered one time and the one or more therapeutic agents and/or active agents can be administered one or more times. Dosing schedules can be determined by skilled person in the art. In an aspect of a disclosed method of producing HDAC11-activated macrophages, the subject's physiology and/or functionality can be restored to normal or near normal levels (e.g., with little or no inflammation or little or no signs and/or symptoms of inflammation).

In an aspect of a disclosed method of producing HDAC11-activated macrophages, the disclosed subject's behavior and/or physiology can be modulated.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, a disclosed method of producing HDAC11-activated macrophages can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof. In an aspect, modifying the administering step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof.

In an aspect, a disclosed method of producing HDAC11-activated macrophages can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer HDAC11-activated M2 macrophages or a composition comprising HDAC11-activated M2 macrophages. In an aspect, metabolic and/or physiologic data can inform the clinician when make subsequent treatment decisions.

### 2. Methods of Treating a Subject

Disclosed herein is a method of treating a subject, the method comprising administering to a subject in need thereof a therapeutically effective amount of HDAC11-activated M2 macrophages or a composition comprising HDAC11-activated M2 macrophages. Disclosed herein is a method of treating a subject, the method comprising administering to a subject in need thereof a therapeutically effective amount of HDAC11-activated M2 macrophages or a composition comprising HDAC11-activated M2 macrophages, wherein following the administering step, inflammation in one or more of the subject's body systems is decreased and/or diminished. Disclosed herein is a method of treating a subject, the method comprising administering to a subject in need thereof a therapeutically effective amount of HDAC11-activated M2 macrophages or a composition comprising HDAC11-activated M2 macrophages, thereby decreasing and/or diminishing inflammation in one or more of the subject's body systems. Disclosed herein is a method of treating a subject, the method comprising administering to a subject in need thereof a therapeutically effective amount of HDAC11-activated M2 macrophages or a composition comprising HDAC11-activated M2 macrophages, and decreasing and/or diminishing inflammation in one or more of the subject's body systems.

In an aspect, a disclosed method of treating a subject can further comprise generating HDAC11-activated and M2 polarized macrophages. In an aspect, generating HDAC11-activated and M2 polarized macrophages can be an ex vivo process. In an aspect, HDAC11-activated macrophages can be any disclosed HDAC11-activated and M2 polarized macrophages. In an aspect, a disclosed method of treating a subject can further comprise activating M0 macrophages or naïve macrophages. In an aspect, activating M0 macrophages or naïve macrophages can comprise contacting a population of naïve macrophages with one or more HDAC11 inhibitors.

In an aspect of a disclosed method of treating a subject, following the contacting of the M0 macrophages or naïve macrophages with HDAC11 inhibitor, the HDAC11-activated macrophages are then polarized towards an anti-inflammatory phenotype. In an aspect of a disclosed method of treating a subject, following the contacting of the M0 macrophages or naïve macrophages with HDAC11 inhibitor, the HDAC11-activated macrophages are then polarized towards a M2 phenotype. In an aspect, a disclosed method of treating a subject can further comprise contacting a population of polarized macrophages with an HDAC11 inhibitor. In an aspect of a disclosed method of treating a subject, following contacting a population of polarized macrophages with an HDAC11 inhibitor, the anti-inflammatory phenotype in enhanced and/or improved.

In an aspect of a disclosed method of treating a subject, following contacting a population of polarized macrophages with an HDAC11 inhibitor, wherein the macrophages are polarized towards an enhanced and/or improved M2 phenotype. In an aspect of a disclosed method of treating a subject, contacting a population of M0 or naïve macrophages with one or more HDAC11 inhibitors can enhance and/or improve the M2 phenotype of the macrophages upon polarization. In an aspect of a disclosed method of treating a subject, contacting a population of polarized macrophages with one or more HDAC11 inhibitors can enhance and/or improve the M2 phenotype of the polarized macrophages. In an aspect of a disclosed method of treating a subject, contacting a population of M0 or naïve macrophages with one or more HDAC11 inhibitors can enhance and/or improve the anti-inflammatory phenotype of the macrophages upon polarization. In an aspect of a disclosed method of treating a subject, contacting a population of polarized macrophages with one or more HDAC11 inhibitors can enhance and/or improve the anti-inflammatory phenotype of the polarized macrophages.

In an aspect, HDAC11-activated macrophages or HDAC11 polarized macrophage can refer to a M0 or naïve macrophage that has been treated ex vivo with an HDAC11 inhibitor (such as, for example a disclosed HDAC11 inhibitor or disclosed selective HDAC11 inhibitor). In an aspect, disclosed HDAC11-activated macrophages are first treated ex vivo with a disclosed HDAC11 inhibitor and then treated ex vivo with a disclosed macrophage polarizing agent. In an aspect, disclosed HDAC11 polarized macrophages are first treated ex vivo with a macrophage polarizing agent and then treated ex vivo with a disclosed HDAC11 inhibitor.

In an aspect of a disclosed method of treating a subject, wherein contacting a population of naïve macrophages with an HDAC11 inhibitor comprises a pre-determined amount of time. In an aspect, for example, contacting a population of naïve macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of naïve macrophages with an HDAC11 inhibitor can comprise more than 24 hours.

In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, contacting a population of polarized macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of polarized macrophages with an HDAC11 inhibitor can comprise more than 24 hours.

In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, naïve macrophages can be contacted with an HDAC11 inhibitor one or more times, and then polarized to M2 macrophages. In an aspect, polarized macrophages can be contacted with an HDAC11 inhibitor one or more times.

In an aspect of a disclosed method of treating a subject, a disclosed subject in need thereof can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, a disclosed subject can have one or more foot ulcers. In an aspect, a disclosed subject can have diabetes. In an aspect, a disclosed subject can be the recipient of one or more solid organ transplants. In an aspect, a disclosed subject can have irritable bowel disease (IBD). In an aspect, a disclosed subject can have one or more inflammatory diseases. In an aspect, a disclosed subject can have one or more skin inflammatory diseases. In an aspect, a disclosed subject can have suffered one or more burns. In an aspect, a disclosed subject can have incurred an ischemic injury. In an aspect, a disclosed ischemic injury can affect the subject's brain and/or the subject's heart. In an aspect, a disclosed subject can have suffered one or more myocardial infarctions. In an aspect, a disclosed subject can have suffered one or more transient ischemic attacks.

In an aspect, a disclosed subject can have an autoinflammatory disease (AIF). In an aspect, a disclosed autoinflammatory disease can comprise familial Mediterranean fever (FMF), hyper IgD syndrome (HIDS), tumor necrosis factor receptor associated autoinflammatory syndrome (TRAPS), cryopyrin associated periodic syndromes (CAPS), or Blau syndrome and pyogenic sterile arthritis pyoderma gangrenosum and acne syndrome (PAPA).

In an aspect of a disclosed method of treating a subject, a disclosed subject can have an autoimmune disease (AD). In an aspect, a disclosed autoimmune disease or disorder can comprise *Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, *Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), *Autoimmune hyperlipidemia, *Autoimmune hypophysitis, *Autoimmune inner car disease (AIED), *Autoimmune lymphoproliferative syndrome (ALPS), *Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, *Autoimmune retinopathy (AIR), *Autoimmune sudden sensorineural hearing loss (SNHL), Balo disease, Behçet's disease, *Birdshot chorioretinopathy/birdshot uveitis, Bullous pemphigoid, *Castleman disease, Celiac disease, *Chagas disease, Chronic inflammatory demyelinating polyneuropathy (CIDP), *Chronic urticaria (CU), Churg-Strauss syndrome/cosinophilic granulomatosis with polyangiitis (EGPA), Cogan's syndrome, Cold agglutinin disease, CREST syndrome|limited cutaneous systemic sclerosis, Crohn's disease (CD), *Cronkhite-Canada syndrome (CSS), *Cryptogenic organizing pneumonia (COP), Dermatitis herpetiformis, Dermatomyositis, Devic's disease/neuromyelitis optica (NMO), Diabetes, type 1, Discoid lupus, *Dressler's syndrome/postmyocardial infarction/postpericardiotomy syndrome, *Eczema/Atopic Dermatitis, *Endometriosis, *Eosinophilic csophagitis, Eosinophilic fasciitis, *Erythema nodosum, *Essential mixed cryoglobulinemia, Evans syndrome, *Fibrosing alveolitis/Idiopathic pulmonary fibrosis (IPF), *Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barré syndrome (GBS), Hashimoto's thyroiditis/chronic lymphocytic thyroiditis/autoimmune thyroiditis, Henoch-Schönlein purpura/IgA vasculitis, *Hidradenitis suppurativa, Hurst's disease/acute hemorrhagic leukoencephalitis (AHLE), *Hypogammaglobulinemia, IgA nephropathy/Berger's disease, Immune-mediated necrotizing myopathy (IMNM), Immune thrombocytopenia (ITP)/autoimmune thrombocytopenia purpura/autoimmune thrombocytopenia, *Inclusion body myositis, *IgG4-related sclerosing disease (ISD), *Interstitial cystitis, Juvenile idiopathic arthritis/Adult-onset Still's disease, Juvenile polymyositis|Juvenile dermatomyositis|juvenile myositis, *Kawasaki disease, Lambert-Eaton myasthenic syndrome (LEMS), *Leukocytoclastic vasculitis, *Lichen planus, *Lichen sclerosus, *Ligneous conjunctivitis, Linear IgA disease (LAD)|linear IgA bullous dermatosis (LABD), Lupus nephritis, *Lyme disease/chronic Lyme disease/post-treatment Lyme disease syndrome (PTLDS), *Lymphocytic colitis/microscopic colitis, Ménière's disease, *Microscopic polyangiitis (MPA)/ANCA-associated vasculitis, Mixed connective tissue disease (MCTD), *Mooren's ulcer, *Mucha-Habermann disease, *Multifocal motor neuropathy, Multiple sclerosis (MS), *Myalgic encephalomyelitis (ME)/Chronic fatigue syndrome (CFS), Myasthenia gravis (MG), *Narcolepsy, Ocular cicatricial pemphigoid, *Opsoclonus-myoclonus syndrome (OMS), Palindromic rheumatism, Parancoplastic cerebellar degeneration, Parancoplastic pemphigus, *Parry-Romberg syndrome (PRS)/Hemifacial atrophy (HFA)/Progressive facial hemiatrophy, Paroxysmal nocturnal hemoglobinuria (PNH), *Peripheral uveitis/pars planitis, *PANS/PANDAS, Parsonage-Turner syndrome, Pemphigus gestationis/herpes gestationis, Pemphigus foliaceus, Pemphigus vulgaris, *Pernicious anemia, POEMS syndrome, Polyarteritis nodosa, Polymyalgia rheumatica, Polymyositis, *Postural orthostatic tachycardia syndrome (POTS), Primary biliary cirrhosis (PBC)/primary biliary cholangitis, *Primary sclerosing cholangitis (PSC), Psoriasis, Palmoplantar Pustulosis, Psoriatic arthritis, *Pulmonary fibrosis, idiopathic (IPF), Pure red cell aplasia (PRCA), *Pyoderma gangrenosum, Raynaud's syndrome/phenomenon, Reactive arthritis/Reiter's syndrome, *Reflex sympathetic dystrophy syndrome (RSD)/Complex regional pain syndrome (CRPS), Relapsing polychondritis, *Restless leg syndrome (RLS)/Willis-Ekbom disease, Rheumatic fever, Rheumatoid arthritis, Sarcoidosis, Schmidt syndrome/autoimmune polyendocrine syndrome type II, *Scleritis, Scleroderma, *Serpiginous choroidopathy, Sjögren's syndrome, *Stiff person syndrome (SPS), Small fiber sensory neuropathy, Systemic lupus erythematosus (SLE), *Subacute bacterial endocarditis (SBE), *Susac syndrome, *Sydenham's chorea, *Sympathetic ophthalmia, *Takayasu's arteritis (vasculitis), Testicular autoimmunity (vasculitis, orchitis), *Tolosa-Hunt syndrome, *Transverse myelitis (TM), *Tubulointerstitial nephritis uveitis syndrome (TINU), Ulcerative colitis (UC), Undifferentiated connective tissue disease (UCTD), *Uveitis|anterior/intermediate/posterior, *Vasculitis, Vitiligo, *Vogt-Koyanagi-Harada syndrome (VKH), or any combination thereof.

In an aspect of a disclosed method of treating a subject, a disclosed subject can be male or female. In an aspect, a subject can be an adult, a teenager, an adolescent, a child, a toddler, a baby, or an infant. In an aspect, a subject can have received treatment for one or more disclosed AIFs or ADs.

In an aspect of a disclosed method of treating a subject, a subject can be treatment-naïve.

In an aspect, a disclosed method of treating a subject can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample can be collected from a subject prior to treatment, during treatment, after treatment, or any combination thereof. In an aspect, a blood sample can be collected from a subject at any time deemed medically and/or clinically appropriate by the skilled clinician.

In an aspect, a disclosed method of treating a subject can further comprise isolating monocytes from peripheral blood monocular cells in the subject's blood sample. In an aspect, a disclosed method of treating a subject can further comprise isolating bone marrow derived monocytes from the subject's blood sample. In an aspect, a disclosed method of treating a subject can further comprise isolating monocytes from the subject's blood sample. In an aspect, a disclosed method of treating a subject can further comprise isolating naïve macrophages (M0) from the subject's blood sample. In an aspect, a disclosed method of treating a subject can further comprise subjecting a disclosed blood sample to centrifugation. In an aspect, a disclosed method can further comprise separating the blood sample into its component parts using, for example, centrifugation, apheresis, or any technique to the skilled person. In an aspect, a disclosed separating step can comprise generating a layer of clear fluid, a layer of red fluid, and a thin layer in between the clear fluid layer and the red fluid layer. In an aspect, a disclosed red layer can comprise red blood cells. In an aspect, a disclosed clear layer can comprise plasma. In an aspect, a disclosed thin layer in between the red layer and the clear layer can comprise the buffy coat. In an aspect, a disclosed buffy coat can comprise white blood cells and platelets.

In an aspect, a disclosed method can further comprise isolating peripheral blood mononuclear cells (PMBCs) from the buffy coat. In an aspect, PMBCs can comprise lymphocytes and/or monocytes. In an aspect, macrophages can be derived from monocytes. In an aspect, isolating monocytes can be done by any method and/or technique known to the skilled person.

For example, in an aspect, a conical blood filter and the buffy coat having approximately 10 mL and 70 mL respectively, can be used. In an aspect, a disclosed sample can be collected in a tube, e.g., a 50 mL tube. In an aspect, if using a conical blood filter, then dilute the sample by adding PBS, for example, at room temperature. In an aspect, a disclosed method can add 10 mL of Ficoll-Hypaque-1077 (or Lymphoprep) to a disclosed centrifuge tube at room temperature. In an aspect, a disclosed method can comprise carefully layering the blood (e.g., 3 mL) onto the Ficoll-Hypaque-1077. In an aspect, blood can stay in the upper layer and the Ficoll-Hypaque 1077 in the lowest one. In an aspect, a disclosed method can comprise centrifuging at 400×g for about 30 min at room temperature. In an aspect of a disclosed method, the centrifuge can be set for 0 (zero) acceleration and 0 (zero) break. In an aspect, after centrifugation, 3 layers can be visible. In an aspect, an upper layer can comprise plasma and the Ficoll-Hypaque. In an aspect, an opaque interface can comprise the mononuclear cells. In an aspect, a lower lever can comprise the red blood cells. In an aspect of a disclosed method, a Pasteur pipette can be used to aspirate the upper layer until close to the opaque phase comprising the mononuclear cells. In an aspect, the upper layer can be discarded. In an aspect of a disclosed method, the disclosed opaque interface can be transferred to a tube (e.g., 50 mL). In an aspect, a tube can be filled with cold PBS. In an aspect of a disclosed method can comprise centrifuged at 250×g for about 10 min at 4° C. In an aspect, a disclosed centrifuge can be at full acceleration and break 5. In an aspect of the disclosed method, at the end of the first washing, the supernatant can appear turbid due to presence of platelets. In an aspect of a disclosed method, the supernatant can be aspirated and the pellet can be resuspended with cold PBS. In an aspect of a disclosed method, centrifuging can be at 250×g for about 10 min at 4° C. In an aspect of a disclosed method, washing steps can be repeated 3-4 times or can be repeated until a clear supernatant is obtained. In an aspect of a disclosed method, monocytes can be isolated by negative selection and/or cell adhesion. Both techniques are known to the skilled person in the art.

In an aspect, a disclosed method of treating a subject can further comprise isolating resting or M0 macrophages from the buffy coat.

In an aspect, a disclosed method of treating a subject can further comprise differentiating monocytes into resting or M0 macrophages. In an aspect of a disclosed method of treating a subject, disclosed macrophages can be resting or M0 macrophages. In an aspect, a disclosed method of treating a subject can further comprise polarizing the resting or M0 macrophages into a M2 phenotype or anti-inflammatory phenotype. In an aspect, a disclosed method of treating a subject can further comprise polarizing the resting or M0 macrophages into an alternatively activated macrophage phenotype. In an aspect, a disclosed method of treating a subject can further comprise polarizing the resting or M0 macrophages into a M2 phenotype or anti-inflammatory phenotype comprises contacting the resting or M0 macrophages with IL-4, IL-10, IL-13, TGFβ-1, PGE2, or any combination thereof. In an aspect of a disclosed method of treating a subject, resting or M0 macrophages can be polarized into a classically activated macrophage phenotype.

In an aspect of a disclosed method of treating a subject, resting or M0 macrophages can be polarized into a M2 phenotype or anti-inflammatory phenotype. In an aspect of a disclosed method of treating a subject, resting or M0 macrophages can be polarized into an alternatively activated macrophage phenotype. In an aspect of a disclosed method of treating a subject, resting or M0 macrophages can be polarized into a M2 phenotype or anti-inflammatory phenotype comprises contacting the resting or M0 macrophages with IL-4, IL-10, IL-13, TGFβ-1, PGE2, or any combination thereof.

In an aspect of a disclosed method of treating a subject, polarizing resting or M0 macrophages into a M2 phenotype or an anti-inflammatory phenotype can comprise about a 1-day to about a 10-day incubation. In an aspect of a disclosed method of treating a subject, polarizing resting or M0 macrophages into a M2 phenotype or an anti-inflammatory phenotype can comprise about a 3-day to about a 7-day incubation. In an aspect of a disclosed method of treating a subject, polarizing the resting or M0 macrophages into a M2 phenotype or an anti-inflammatory phenotype can comprise about a 1-day incubation, about a 2-day incubation, about a 3-day incubation, about a 4-day incubation, about a 5-day incubation, about a 6-day incubation, about a 7-day incubation, about a 8-day incubation, about a 9-day incubation, about a 10-day incubation, or more than a 10-day incubation. In an aspect of a disclosed method of treating a subject, wherein following the contacting of the resting or M0 macrophages with a disclosed HDAC11 inhibitor, polarizing macrophages can be incubated from about 1 day to about 10 days, or from about 3 days to about 7 days, or for about 1 day, about 2 days, about 3 days, about 4 days, about 5 days, about 6 days, about 7 days, about 8 days, about 9 days, about 10 days, or more than 10 days. In an aspect, one or more steps of a disclosed method of treating a subject can be performed ex vivo.

In an aspect, a disclosed method of treating a subject can comprise one or more disclosed HDAC11 inhibitors or a composition comprising one or more disclosed HDAC11 inhibitors. In an aspect, the one or more disclosed HDAC11 inhibitors or a composition comprising one or more disclosed HDAC11 inhibitors can be administered one or more times. In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect of a disclosed method of treating a subject, a disclosed HDAC11 inhibitor can comprise elevenostat (JB3-22), garcinol, SIS7, SIS17, FT895, romidepsin, quisinostat, or any combination thereof. In an aspect of a disclosed method of treating a subject, a disclosed HDAC11 inhibitor can comprise any commercially available HDAC11 inhibitor or a combination of commercially available HDAC11 inhibitors. In an aspect, an HDAC11 inhibitor can comprise a selective HDAC11 inhibitor or HDAC11 selective inhibitor and can refer to a compound that preferentially inhibits histone deacetylase 11 over one or more other histone deacetylase isoforms, e.g., HDAC1, HDAC2, HDAC3, HDAC4, HDAC5, HDAC6, HDAC7, HDAC8, HDAC9, and/or HDAC10 in a cell-based in vitro assay. For example, a compound having a HDAC11 IC_{SO}=5 nM and a HDAC1 IC_{SO} of 500 nM is a selective HDAC11 inhibitor that is 100-fold more selective over HDAC1; a compound having a HDAC11 IC_{SO}=5 nM, a HDAC1 IC_{SO}=500 nM, and a HDAC3 IC_{SO}=50 nM is a selective HDAC11 inhibitor that is 100-fold more selective over HDAC1 and 10-fold more selective over HDAC3; and so on. In one aspect, a selective HDAC11 inhibitor can preferentially inhibit HDAC11 over HDAC1, HDAC2, HDAC3, HDAC4, HDAC5, HDAC6, HDAC7, HDAC8, HDAC9, and/or HDAC10. In an aspect, the selective HDAC11 inhibitor can preferentially inhibit HDAC11 over one or more other HDAC isoforms.

In an aspect, the selective HDAC11 inhibitor can be at least about 5-fold more selective over one or more other HDAC isoforms. In an aspect, the selective HDAC11 inhibitor can be at least about 10-fold more selective over one or more other HDAC isoforms. In an aspect, the selective HDAC11 inhibitor can be at least about 15-fold more selective over one or more other HDAC isoforms. In aspect, the selective HDAC11 inhibitor can be at least about 20-fold more selective over one or more other HDAC isoforms. In an aspect, the selective HDAC11 inhibitor can be at least about 30-fold more selective over one or more other HDAC isoforms. In an aspect, the selective HDAC11 inhibitor can be at least about 40-fold more selective over one or more other HDAC isoforms. In an aspect, the selective HDAC11 inhibitor can be at least about 50-fold more selective over one or more other HDAC isoforms. In an aspect, the selective HDAC6 inhibitor can be at least about 100-fold more selective over one or more other HDAC isoforms. In another aspect, the selective HDAC11 inhibitor can be at least about 150-fold more selective over one or more other HDAC isoforms. In an aspect, the selective HDAC11 inhibitor can be at least about 200-fold more selective over one or more other HDAC isoforms. In an aspect, the selective HDAC11 inhibitor can be at least about 250-fold more selective over one or more other HDAC isoforms. In an aspect, the selective HDAC11 inhibitor can be at least about 500-fold more selective over one or more other HDAC isoforms. In an aspect, the selective HDAC11 inhibitor can be at least about 750-fold more selective over one or more other HDAC isoforms. In an aspect, the selective HDAC11 inhibitor can be at least about 1000-fold more selective over one or more other HDAC isoforms. In an aspect, the selective HDAC11 inhibitor can be at least about 2000-fold more selective over one or more other HDAC isoforms. In an aspect, the selective HDAC11 inhibitor can be at least about 3000-fold more selective over one or more other HDAC isoforms.

HDAC11 selectivity over the other HDAC isoforms in cell-based assays can be determined using methods known in the art. In another aspect, the selective HDAC11 inhibitor can be at about 10-fold to about 3000-fold more selective over one or more other HDAC isoforms. In an aspect, the selective HDAC11 inhibitor can be at about 20-fold to about 3000-fold more selective over one or more other HDAC isoforms. In an aspect, the selective HDAC11 inhibitor can be at about 50-fold to about 3000-fold more selective over one or more other HDAC isoforms. In an aspect, the selective HDAC11 inhibitor can be at about 100-fold to about 3000-fold more selective over one or more other HDAC isoforms. In an aspect, the selective HDAC11 inhibitor can be at about 500-fold to about 3000-fold more selective over one or more other HDAC isoforms.

In an aspect of a disclosed method, about 50 nM to about 25 μM of a disclosed HDAC11 inhibitor can polarize resting or M0 macrophages into a M2 phenotype or an anti-inflammatory phenotype.

In an aspect of a disclosed method of treating a subject, contacting the resting or M0 macrophages with one or more HDAC11 inhibitors can occur prior to polarizing the resting or M0 macrophages into the M2 phenotype or anti-inflammatory phenotype. In an aspect of a disclosed method of treating a subject, contacting the resting or M0 macrophages with one or more HDAC11 inhibitors can occur after polarizing the resting or M0 macrophages into the M2 phenotype or anti-inflammatory phenotype. In an aspect of a disclosed method of treating a subject, contacting the resting or M0 macrophages with one or more HDAC11 inhibitors can occur during the polarizing the resting or M0 macrophages into the M2 phenotype or anti-inflammatory phenotype. In an aspect, a disclosed method of treating a subject can further comprise confirming the resulting macrophages demonstrate the M2 phenotype or anti-inflammatory phenotype. In an aspect, confirming the M2 phenotype or anti-inflammatory phenotype can comprise detecting a change in the expression level of one or more relevant genes and/or relevant proteins. In an aspect of a disclosed method of treating a subject, detecting a change in the expression level can comprise detecting an increase in the expression level of one or more relevant genes and/or relevant proteins. In an aspect, detecting a change in expression level can comprise detecting a decrease in the expression level of one or more relevant genes and/or relevant proteins.

In an aspect of a disclosed method of treating a subject, one or more disclosed relevant genes and/or relevant proteins can comprise IL-10, TGF-β, Fizz1, Arg1, Mrc1 (CD206), or any combination thereof. In an aspect, detecting a change in the expression level can comprise detecting an increase in the expression level of IL-10, TGF-β, Fizz1, Arg1, Mrc1 (CD206), or any combination thereof. In an aspect, a disclosed increase can comprise a 10%, 20%, 30%, 40%, 50%, 60%, 70%, 80%, 90%, 100%, or any amount of an increase when compared to a control subject (such as, for example, a subject that has not received HDAC11-activated M2 macrophages or a composition comprising HDAC11-activated M2 macrophages). In an aspect, a disclosed increase can comprise a 10-20%, 20-30%, 30-40%, 40-50%, 50-60%, 60-70%, 70-80%, 80-90%, or 90-100% or any amount of an increase when compared to a control subject (such as a subject that has not received HDAC11-activated M2 macrophages or a composition comprising HDAC11-activated M2 macrophages).

In an aspect, a disclosed decrease reduction can comprise a 10%, 20%, 30%, 40%, 50%, 60%, 70%, 80%, 90%, 100%, or any amount of a decrease when compared to a control subject (such as, for example, a subject that has not received HDAC11-activated M2 macrophages or a composition comprising HDAC11-activated M2 macrophages). In an aspect, a disclosed decrease can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated M2 macrophages or a composition comprising HDAC11-activated M2 macrophages).

In an aspect of a disclosed method of treating a subject, polarized M2 macrophages can be cryopreserved. In an aspect, cryopreserved polarized M2 macrophages can be thawed. In an aspect, thawing cryopreserved polarized M2 macrophages can be done by any technique known to the skilled person in the art. In an aspect, thawed polarized M2 macrophages can be administered to a subject. In an aspect of a disclosed method of treating a subject, resting or M0 macrophages can be cryopreserved prior to polarization and/or activation. In an aspect, cryopreserved resting or M0 macrophages can be thawed and then can be polarized and/or activated. In an aspect, thawing cryopreserved resting or M0 macrophages can be done by any technique known to the skilled person in the art. In an aspect, thawed resting or M0 macrophages can be polarized and/or activated and can then be administered to a subject. In an aspect, administering can comprise intravenous administration of HDAC11-activated M2 macrophages or a composition comprising HDAC11-activated M2 macrophages. In an aspect, administering can comprise systemic administration or local administration of HDAC11-activated M2 macrophages or a composition comprising HDAC11-activated M2 macrophages.

In an aspect, local administration can comprise delivery to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof. In an aspect, local delivery or local administration can refer to delivering or administering HDAC11-activated M2 macrophages or a composition comprising HDAC11-activated M2 macrophages directly to a target site within a subject. For example, HDAC11-activated M2 macrophages or a composition comprising HDAC11-activated M2 macrophages can be locally delivered by direct injection into an affected body system (such as one, for example, having inflammation).

Local delivery or local administration can also include topical applications or localized injection techniques such as intramuscular, subcutaneous, and/or intradermal injection of HDAC11-activated M2 macrophages or a composition comprising HDAC11-activated M2 macrophages. In an aspect, local administration of HDAC11-activated M2 macrophages or a composition comprising HDAC11-activated M2 macrophages can comprise delivery to one or more body systems in need thereof. In an aspect, local administration or local delivery HDAC11-activated M2 macrophages or a composition comprising HDAC11-activated M2 macrophages can comprise a transdermal administration, administration via implant, administration via suppository, implantation via a biodegradable polymer or matrix, implantation via a biodegradable hydrogel, or any combination thereof.

In an aspect of a disclosed method of treating a subject, administering HDAC11-activated M2 macrophages or a composition comprising HDAC11-activated M2 macrophages can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, HDAC11-activated M2 macrophages or a composition comprising HDAC11-activated M2 macrophages can be administered via multiple routes of administration. In an aspect of a disclosed method of treating a subject, following administration of HDAC11-activated M2 macrophages or a composition comprising HDAC11-activated M2 macrophages to the subject, inflammation in one or more of the subject's body systems can be diminished and/or decreased. In an aspect of a disclosed method of treating a subject, the decrease in inflammation in one or more of the subject's body systems can be measured objectively and/or subjectively.

In an aspect of a disclosed method of treating a subject, following administration of HDAC11-activated M2 macrophages or a composition comprising HDAC11-activated M2 macrophages to the subject, the pathology of the subject's disease or disorder can be diminished and/or decreased. In an aspect of a disclosed method of treating a subject, wherein, following administration of HDAC11-activated M2 macrophages or a composition comprising HDAC11-activated M2 macrophages to the subject, the subject's symptoms can be diminished and/or decreased.

In an aspect of a disclosed method of treating a subject, wherein, following administration of HDAC11-activated M2 macrophages or a composition comprising HDAC11-activated M2 macrophages to the subject, the subject's quality of life can be improved and/or enhanced. In an aspect, quality of life can be measured objectivity and/or subjectively. In an aspect of a disclosed method of treating a subject, wherein, following administration of HDAC11-activated M2 macrophages or a composition comprising HDAC11-activated M2 macrophages to the subject, the one or more transplanted organs can be not rejected.

In an aspect of a disclosed method of treating a subject, wherein, following administration of HDAC11-activated M2 macrophages or a composition comprising HDAC11-activated M2 macrophages to the subject, the one or more transplanted organs can be spared from rejection.

In an aspect of a disclosed method of treating a subject, wherein, following administration of HDAC11-activated M2 macrophages or a composition comprising HDAC11-activated M2 macrophages to the subject, the healing of the subject's one or more chronic wounds can be improved and/or enhanced. In an aspect of a disclosed method of treating a subject, wherein improving and/or enhancing healing of one or more chronic wounds can comprise improving angiogenesis, innervation, cellular migration, or any combination thereof. In an aspect of a disclosed method of treating a subject, wherein following administration of HDAC11-activated M2 macrophages or a composition comprising HDAC11-activated M2 macrophages to the subject, one or more of the subject's body systems can experience and/or show signs of cellular regeneration and/or cellular repair.

In an aspect of a disclosed method of treating a subject, wherein the one or more body systems experiencing and/or showing signs of regeneration and/repair can comprise the cardiovascular system. In an aspect of a disclosed method of treating a subject, wherein previously ischemic and/or infarcted and/or languishing areas of the cardiovascular system can show signs of regeneration and/or repair. In an aspect of a disclosed method of treating a subject, the one or more body systems experiencing and/or showing signs of regeneration and/repair can comprise the central nervous system and/or peripheral nervous system. In an aspect of a disclosed method of treating a subject, wherein previously ischemic and/or infarcted/or languishing areas of the central nervous system and/or peripheral nervous system can show signs of regeneration and/or repair. In an aspect of a disclosed method of treating a subject, wherein the one or more body systems experiencing and/or showing signs of regeneration and/repair can comprise the integumentary system. In an aspect of a disclosed method of treating a subject, wherein the one or more body systems experiencing and/or showing signs of regeneration and/repair can comprise the digestive system. In an aspect, a disclosed method of treating a subject can further comprise diminishing and/or decreasing inflammation in one or more of the subject's body systems. Body systems are disclosed supra.

In an aspect, a decrease in inflammation in one or more of the subject's body systems can be measured objectively and/or subjectively. In an aspect, a disclosed method of treating a subject can further comprise diminishing and/or decreasing the pathology of the subject's disease or disorder. In an aspect, a disclosed method of treating a subject can further comprise diminishing and/or decreasing the subject's symptoms. In an aspect, a disclosed method of treating a subject can further comprise improving and/or enhancing the subject's quality of life. In an aspect, quality of life can be measured objectivity and/or subjectively. In an aspect, a disclosed method of treating a subject can further comprise preventing the rejection of one or more transplanted organs in the subject. In an aspect, a disclosed method of treating a subject can further comprise minimizing the risk of rejection of one or more transplanted organs in the subject. In an aspect, a disclosed method of treating a subject can further comprise improving and/or enhancing the healing of one or more chronic wounds in the subject. In an aspect, a disclosed method of treating a subject can further comprise improving and/or enhancing healing of one or more chronic wounds. In an aspect, a disclosed method of treating a subject can further comprise improving and/or enhancing angiogenesis, innervation, cellular migration, or any combination thereof in and/or around one or more chronic wounds in the subject.

In an aspect, a disclosed method of treating a subject can further comprise improving and/or enhancing cellular regeneration and/or cellular repair or one or more of the subject's affected body systems. In an aspect, a disclosed method of treating a subject can further comprise improving and/or enhancing cellular regeneration and/or cellular repair in the subject's central nervous system and/or peripheral nervous system. In an aspect, a disclosed method of treating a subject can further comprise improving and/or enhancing cellular regeneration and/or cellular repair in the subject's integumentary system. In an aspect, a disclosed method of treating a subject can further comprise improving and/or enhancing cellular regeneration and/or cellular repair in the subject's digestive system.

In an aspect, a disclosed method of producing HDAC11-activated macrophages can further comprise repeating one or more times one or more steps of a disclosed method. In an aspect of a disclosed method of treating a subject, a disclosed obtaining step can be repeated, a disclosed isolating step can be repeated, a disclosed contacting step can be repeated, a disclosed activating step can be repeated, a disclosed polarizing step can be repeated, a disclosed incubating step can be repeated, a disclosed administering step can be repeated, or any combination thereof. In an aspect, a disclosed method of treating a subject can further comprise repeating an obtaining step, repeating an isolating step, repeating a contacting step, repeating an activating step, repeating a polarizing step, repeating an incubating step, repeating an administering step, or repeating one or more time any combination of steps thereof.

In an aspect of a disclosed method of treating a subject, a disclosed improvement and/or enhancement (such as, for example, in the subject's quality of life and/or movement and/or would healing and/or angiogenesis, etc.) can comprise a 10%, 20%, 30%, 40%, 50%, 60%, 70%, 80%, 90%, 100%, or any amount of improvement and/or enhancement when compared to a control subject (such as, for example, a subject that has not received HDAC11-activated M2 macrophages or a composition comprising HDAC11-activated M2 macrophages). In an aspect, a disclosed improvement and/or enhancement (such as, for example, in the subject's quality of life and/or movement and/or would healing and/or angiogenesis, etc.) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not HDAC11-activated M2 macrophages or a composition comprising HDAC11-activated M2 macrophages).

In an aspect of a disclosed method of treating a subject, a disclosed diminishment (such as, for example, in the subject's pain and/or wound size and/or restrictions) can comprise a 10%, 20%, 30%, 40%, 50%, 60%, 70%, 80%, 90%, 100%, or any amount of a decrease when compared to a control subject (such as, for example, a subject that has not received HDAC11-activated M2 macrophages or a composition comprising HDAC11-activated M2 macrophages)). In an aspect, a disclosed diminishment (such as, for example, in the subject's pain and/or wound size and/or restrictions) can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated M2 macrophages or a composition comprising HDAC11-activated M2 macrophages).

In an aspect, a disclosed method of treating a subject can further comprise administering to a disclosed subject one or more therapeutic agents and/or active agents. In an aspect, one or more disclosed therapeutic agents and/or active agents can comprise an immune modulator. In an aspect, a disclosed immune modulator can refer to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions.

In an aspect of a disclosed method of treating a subject, administering can comprise intravenous administration of one or more therapeutic agents and/or active agents. In an aspect, administering can comprise systemic administration or local administration of one or more therapeutic agents and/or active agents.

In an aspect of a disclosed method of treating a subject, local delivery or local administration can refer to delivering or administering one or more therapeutic agents and/or active agents directly to a target site within a subject. For example, the one or more therapeutic agents and/or active agents can be locally delivered by direct injection into an affected body system (such as one, for example, having inflammation). Local delivery or local administration can also include topical applications or localized injection techniques such as intramuscular, subcutaneous, and/or intradermal injection of one or more therapeutic agents and/or active agents. In an aspect, local administration of one or more therapeutic agents and/or active agents can comprise delivery to one or more body systems in need thereof. In an aspect, local administration or local delivery of one or more therapeutic agents and/or active agents can comprise a transdermal administration, administration via implant, administration via suppository, implantation via a biodegradable polymer or matrix, implantation via a biodegradable hydrogel, or any combination thereof. A skilled clinician can determine the best route of administration for a subject at a given time.

In an aspect of a disclosed method of treating a subject, HDAC11-activated M2 macrophages or a composition comprising HDAC11-activated M2 macrophages can be administered to the subject prior to, concurrent with, or after administration of one or more therapeutic agents and/or active agents. In an aspect of a disclosed method of treating a subject, HDAC11-activated M2 macrophages or a composition comprising HDAC11-activated M2 macrophages can be administered one or more times and the one or more therapeutic agents and/or active agents can be administered one or more times. In an aspect of a disclosed method of treating a subject, HDAC11-activated M2 macrophages or a composition comprising HDAC11-activated M2 macrophages can be administered one time and the one or more therapeutic agents and/or active agents can be administered one or more times. Dosing schedules can be determined by skilled person in the art. In an aspect of a disclosed method of treating a subject, the subject's physiology and/or functionality can be restored to normal or near normal levels (e.g., with little or no inflammation or little or no signs and/or symptoms of inflammation).

In an aspect of a disclosed method of treating a subject, the disclosed subject's behavior and/or physiology can be modulated.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, a disclosed method of treating a subject can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method of treating a subject can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect of a disclosed method of treating a subject, modifying the treating step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof. In an aspect of a disclosed method of treating a subject, modifying the administering step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof.

In an aspect, a disclosed method of treating a subject can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method of treating a subject, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer HDAC11-activated M2 macrophages or a composition comprising HDAC11-activated M2 macrophages. In an aspect, metabolic and/or physiologic data can inform the clinician when make subsequent treatment decisions.

Disclosed herein is a method of treating a subject, the method comprising isolating M0 or naïve macrophages from a subject in need thereof, contacting the M0 or naïve macrophages ex vivo with a one or more HDAC11 inhibitors to produce HDAC11-activated macrophage, polarizing the HDAC11-activated macrophages to an anti-inflammatory phenotype, and administering to the subject the HDAC11-activated and polarized macrophages.

Disclosed herein is a method of treating a subject, the method comprising isolating M0 or naïve macrophages from a subject in need thereof, polarizing the M0 or naïve macrophages to an anti-inflammatory phenotype ex vivo, contacting the polarized macrophages with a one or more HDAC11 inhibitors to produce HDAC11-activated macrophage, and administering to the subject the HDAC11-activated and polarized macrophages.

### 3. Methods of Treating a Subject

Disclosed herein is a method of treating a subject, the method comprising administering to a subject in need thereof a therapeutically effective amount of one or more HDAC11 inhibitors or a composition comprising a therapeutically effective amount of one or more HDAC11 inhibitors. Disclosed herein is a method of treating a subject, the method comprising administering to a subject in need thereof a therapeutically effective amount of one or more HDAC11 inhibitors or a composition comprising a therapeutically effective amount of one or more HDAC11 inhibitors, wherein following the administering step, inflammation in one or more of the subject's body systems is decreased and/or diminished. Disclosed herein is a method of treating a subject, the method comprising administering to a subject in need thereof a therapeutically effective amount of one or more HDAC11 inhibitors or a composition comprising a therapeutically effective amount of one or more HDAC11 inhibitors, thereby decreasing and/or diminishing inflammation in one or more of the subject's body systems. Disclosed herein is a method of treating a subject, the method comprising administering to a subject in need thereof a therapeutically effective amount of one or more HDAC11 inhibitors or a composition comprising a therapeutically effective amount of one or more HDAC11 inhibitors, and decreasing and/or diminishing inflammation in one or more of the subject's body systems.

In an aspect of a disclosed method of treating a subject, a disclosed HDAC11 inhibitor can comprise elevenostat (JB3-22), garcinol, SIS7, SIS17, FT895, romidepsin, quisinostat, or any combination thereof. In an aspect of a disclosed method of treating a subject, a disclosed HDAC11 inhibitor can comprise any commercially available HDAC11 inhibitor or a combination of commercially available HDAC11 inhibitors. In an aspect, an HDAC11 inhibitor can comprise a selective HDAC11 inhibitor or HDAC11 selective inhibitor and can refer to a compound that preferentially inhibits histone deacetylase 11 over one or more other histone deacetylase isoforms, e.g., HDAC1, HDAC2, HDAC3, HDAC4, HDAC5, HDAC6, HDAC7, HDAC8, HDAC9, and/or HDAC10 in a cell-based in vitro assay. For example, a compound having a HDAC11 IC_{SO}=5 nM and a HDAC1 IC_{SO} of 500 nM is a selective HDAC11 inhibitor that is 100-fold more selective over HDAC1; a compound having a HDAC11 IC_{SO}=5 nM, a HDAC1 IC_{SO}=500 nM, and a HDAC3 IC_{SO}=50 nM is a selective HDAC11 inhibitor that is 100-fold more selective over HDAC1 and 10-fold more selective over HDAC3; and so on. In one aspect, a selective HDAC11 inhibitor can preferentially inhibit HDAC11 over HDAC1, HDAC2, HDAC3, HDAC4, HDAC5, HDAC6, HDAC7, HDAC8, HDAC9, and/or HDAC10. In an aspect, the selective HDAC11 inhibitor can preferentially inhibit HDAC11 over one or more other HDAC isoforms.

In an aspect, the selective HDAC11 inhibitor can be at least about 5-fold more selective over one or more other HDAC isoforms. In an aspect, the selective HDAC11 inhibitor can be at least about 10-fold more selective over one or more other HDAC isoforms. In an aspect, the selective HDAC11 inhibitor can be at least about 15-fold more selective over one or more other HDAC isoforms. In aspect, the selective HDAC11 inhibitor can be at least about 20-fold more selective over one or more other HDAC isoforms. In an aspect, the selective HDAC11 inhibitor can be at least about 30-fold more selective over one or more other HDAC isoforms. In an aspect, the selective HDAC11 inhibitor can be at least about 40-fold more selective over one or more other HDAC isoforms. In an aspect, the selective HDAC11 inhibitor can be at least about 50-fold more selective over one or more other HDAC isoforms. In an aspect, the selective HDAC6 inhibitor can be at least about 100-fold more selective over one or more other HDAC isoforms. In another aspect, the selective HDAC11 inhibitor can be at least about 150-fold more selective over one or more other HDAC isoforms. In an aspect, the selective HDAC11 inhibitor can be at least about 200-fold more selective over one or more other HDAC isoforms. In an aspect, the selective HDAC11 inhibitor can be at least about 250-fold more selective over one or more other HDAC isoforms. In an aspect, the selective HDAC11 inhibitor can be at least about 500-fold more selective over one or more other HDAC isoforms. In an aspect, the selective HDAC11 inhibitor can be at least about 750-fold more selective over one or more other HDAC isoforms. In an aspect, the selective HDAC11 inhibitor can be at least about 1000-fold more selective over one or more other HDAC isoforms. In an aspect, the selective HDAC11 inhibitor can be at least about 2000-fold more selective over one or more other HDAC isoforms. In an aspect, the selective HDAC11 inhibitor can be at least about 3000-fold more selective over one or more other HDAC isoforms.

HDAC11 selectivity over the other HDAC isoforms in cell-based assays can be determined using methods known in the art. In another aspect, the selective HDAC11 inhibitor can be at about 10-fold to about 3000-fold more selective over one or more other HDAC isoforms. In an aspect, the selective HDAC11 inhibitor can be at about 20-fold to about 3000-fold more selective over one or more other HDAC isoforms. In an aspect, the selective HDAC11 inhibitor can be at about 50-fold to about 3000-fold more selective over one or more other HDAC isoforms. In an aspect, the selective HDAC11 inhibitor can be at about 100-fold to about 3000-fold more selective over one or more other HDAC isoforms. In an aspect, the selective HDAC11 inhibitor can be at about 500-fold to about 3000-fold more selective over one or more other HDAC isoforms.

In an aspect of a disclosed method, about 50 nM to about 25 UM of a disclosed HDAC11 inhibitor can polarize resting or M0 macrophages into a M2 phenotype or an anti-inflammatory phenotype.

In an aspect, a therapeutically effective amount of an HDAC11 inhibitor can comprise from about 50 nM to about 25 UM of an HDAC11-inhibitor. In an aspect, a therapeutically effective amount of an HDAC11-inhibitor can comprise an amount that can elicits and/or can provoke one or more measurable anti-inflammatory responses.

In an aspect, a disclosed method of treating a subject can further comprise administering to the subject HDAC11-activated M2 polarized macrophages, a composition comprising HDAC11-activated M2 polarized macrophages, or a pharmaceutical formulation comprising HDAC11-activated M2 polarized macrophages. In an aspect, a disclosed method of treating a subject can further comprise generating HDAC11-activated M2 polarized macrophages. In an aspect, generating HDAC11-activated M2 polarized macrophages can comprise any method disclosed herein and discussed supra. In an aspect, generating HDAC11-activated M2 polarized macrophages can be an ex vivo process. In an aspect, HDAC11-activated macrophages can be any disclosed HDAC11-activated M2 polarized macrophages.

In an aspect of a disclosed method of treating a subject, a disclosed subject in need thereof can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, a disclosed subject can have one or more foot ulcers. In an aspect, a disclosed subject can have diabetes. In an aspect, a disclosed subject can be the recipient of one or more solid organ transplants. In an aspect, a disclosed subject can have irritable bowel disease (IBD). In an aspect, a disclosed subject can have one or more inflammatory diseases. In an aspect, a disclosed subject can have one or more skin inflammatory diseases. In an aspect, a disclosed subject can have suffered one or more burns. In an aspect, a disclosed subject can have incurred an ischemic injury. In an aspect, a disclosed ischemic injury can affect the subject's brain and/or the subject's heart. In an aspect, a disclosed subject can have suffered one or more myocardial infarctions. In an aspect, a disclosed subject can have suffered one or more transient ischemic attacks.

In an aspect, a disclosed subject can have an autoinflammatory disease (AIF). In an aspect, a disclosed autoinflammatory disease can comprise familial Mediterranean fever (FMF), hyper IgD syndrome (HIDS), tumor necrosis factor receptor associated autoinflammatory syndrome (TRAPS), cryopyrin associated periodic syndromes (CAPS), or Blau syndrome and pyogenic sterile arthritis pyoderma gangrenosum and acne syndrome (PAPA).

In an aspect of a disclosed method of treating a subject, a disclosed subject can have an autoimmune disease (AD). In an aspect, a disclosed autoimmune disease or disorder can comprise *Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, *Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), *Autoimmune hyperlipidemia, *Autoimmune hypophysitis, *Autoimmune inner ear disease (AIED), *Autoimmune lymphoproliferative syndrome (ALPS), *Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, *Autoimmune retinopathy (AIR), *Autoimmune sudden sensorineural hearing loss (SNHL), Balo disease, Behçet's disease, *Birdshot chorioretinopathy/birdshot uveitis, Bullous pemphigoid, *Castleman disease, Celiac disease, *Chagas disease, Chronic inflammatory demyelinating polyneuropathy (CIDP), *Chronic urticaria (CU), Churg-Strauss syndrome/cosinophilic granulomatosis with polyangiitis (EGPA), Cogan's syndrome, Cold agglutinin disease, CREST syndrome|limited cutaneous systemic sclerosis, Crohn's disease (CD), *Cronkhite-Canada syndrome (CSS), *Cryptogenic organizing pneumonia (COP), Dermatitis herpetiformis, Dermatomyositis, Devic's disease/neuromyelitis optica (NMO), Diabetes, type 1, Discoid lupus, *Dressler's syndrome/postmyocardial infarction/postpericardiotomy syndrome, *Eczema/Atopic Dermatitis, *Endometriosis, *Eosinophilic esophagitis, Eosinophilic fasciitis, *Erythema nodosum, *Essential mixed cryoglobulinemia, Evans syndrome, *Fibrosing alveolitis/Idiopathic pulmonary fibrosis (IPF), *Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, *Hidradenitis suppurativa, Hurst's disease/acute hemorrhagic leukoencephalitis (AHLE), *Hypogammaglobulinemia, IgA nephropathy/Berger's disease, Immune-mediated necrotizing myopathy (IMNM), Immune thrombocytopenia (ITP)/autoimmune thrombocytopenia purpura/autoimmune thrombocytopenia, *Inclusion body myositis, *IgG4-related sclerosing disease (ISD), *Interstitial cystitis, Juvenile idiopathic arthritis/Adult-onset Still's disease, Juvenile polymyositis|Juvenile dermatomyositis|juvenile myositis, *Kawasaki disease, Lambert-Eaton myasthenic syndrome (LEMS), *Leukocytoclastic vasculitis, *Lichen planus, *Lichen sclerosus, *Ligneous conjunctivitis, Linear IgA disease (LAD)|linear IgA bullous dermatosis (LABD), Lupus nephritis, *Lyme disease/chronic Lyme disease/post-treatment Lyme disease syndrome (PTLDS), *Lymphocytic colitis/microscopic colitis, Ménière's disease, *Microscopic polyangiitis (MPA)/ANCA-associated vasculitis, Mixed connective tissue disease (MCTD), *Mooren's ulcer, *Mucha-Habermann disease, *Multifocal motor neuropathy, Multiple sclerosis (MS), *Myalgic encephalomyelitis (ME)/Chronic fatigue syndrome (CFS), Myasthenia gravis (MG), *Narcolepsy, Ocular cicatricial pemphigoid, *Opsoclonus-myoclonus syndrome (OMS), Palindromic rheumatism, Parancoplastic cerebellar degeneration, Parancoplastic pemphigus, *Parry-Romberg syndrome (PRS)/Hemifacial atrophy (HFA)/Progressive facial hemiatrophy, Paroxysmal nocturnal hemoglobinuria (PNH), *Peripheral uveitis/pars planitis, *PANS/PANDAS, Parsonage-Turner syndrome, Pemphigus gestationis/herpes gestationis, Pemphigus foliaceus, Pemphigus vulgaris, *Pernicious anemia, POEMS syndrome, Polyarteritis nodosa, Polymyalgia rheumatica, Polymyositis, *Postural orthostatic tachycardia syndrome (POTS), Primary biliary cirrhosis (PBC)/primary biliary cholangitis, *Primary sclerosing cholangitis (PSC), Psoriasis, Palmoplantar Pustulosis, Psoriatic arthritis, *Pulmonary fibrosis, idiopathic (IPF), Pure red cell aplasia (PRCA), *Pyoderma gangrenosum, Raynaud's syndrome/phenomenon, Reactive arthritis/Reiter's syndrome, *Reflex sympathetic dystrophy syndrome (RSD)/Complex regional pain syndrome (CRPS), Relapsing polychondritis, *Restless leg syndrome (RLS)/Willis-Ekbom disease, Rheumatic fever, Rheumatoid arthritis, Sarcoidosis, Schmidt syndrome/autoimmune polyendocrine syndrome type II, *Scleritis, Scleroderma, *Serpiginous choroidopathy, Sjögren's syndrome, *Stiff person syndrome (SPS), Small fiber sensory neuropathy, Systemic lupus crythematosus (SLE), *Subacute bacterial endocarditis (SBE), *Susac syndrome, *Sydenham's chorea, *Sympathetic ophthalmia, *Takayasu's arteritis (vasculitis), Testicular autoimmunity (vasculitis, orchitis), *Tolosa-Hunt syndrome, *Transverse myelitis (TM), *Tubulointerstitial nephritis uveitis syndrome (TINU), Ulcerative colitis (UC), Undifferentiated connective tissue disease (UCTD), *Uveitis|anterior/intermediate/posterior, *Vasculitis, Vitiligo, *Vogt-Koyanagi-Harada syndrome (VKH), or any combination thereof.

In an aspect of a disclosed method of treating a subject, a disclosed subject can be male or female. In an aspect, a subject can be an adult, a teenager, an adolescent, a child, a toddler, a baby, or an infant. In an aspect, a subject can have received treatment for one or more disclosed AIFs or ADs. In an aspect of a disclosed method of treating a subject, a subject can be treatment-naïve.

In an aspect, a therapeutically effective amount of an HDAC11 inhibitor can comprise from about 50 nM to about 25 M of an HDAC11-inhibitor. In an aspect, a therapeutically effective amount of an HDAC11-inhibitor can comprise an amount that can elicits and/or can provoke one or more measurable anti-inflammatory responses.

In an aspect, one or more steps of a disclosed method of treating a subject can be performed ex vivo. In an aspect, a therapeutically effective amount of an HDAC11 inhibitor can comprise from about 50 nM to about 25 μM of an HDAC11-inhibitor. In an aspect, a therapeutically effective amount of an HDAC11-inhibitor can comprise an amount that can elicits and/or can provoke one or more measurable anti-inflammatory responses.

In an aspect, administering a therapeutically effective amount of one or more HDAC11 inhibitors or a composition comprising a therapeutically effective amount of one or more HDAC11 inhibitors can comprise systemic administration or local administration. In an aspect, administering can comprise intravenous administration of HDAC11-activated M2 macrophages or a composition comprising HDAC11-activated M2 macrophages. In an aspect, administering can comprise systemic administration or local administration of HDAC11-activated M2 macrophages or a composition comprising HDAC11-activated M2 macrophages.

In an aspect, local administration can comprise delivery to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect, local delivery or local administration can refer to delivering or administering one or more HDAC11 inhibitors or a composition comprising a one or more HDAC11 inhibitors directly to a target site within a subject. For example, one or more HDAC11 inhibitors or a composition comprising one or more HDAC11 inhibitors can be locally delivered by direct injection into an affected body system (such as one, for example, having inflammation).

In an aspect, local delivery or local administration can refer to delivering or administering HDAC11-activated M2 macrophages or a composition comprising HDAC11-activated M2 macrophages directly to a target site within a subject. For example, HDAC11-activated M2 macrophages or a composition comprising HDAC11-activated M2 macrophages can be locally delivered by direct injection into an affected body system (such as one, for example, having inflammation). Local delivery or local administration can also include topical applications or localized injection techniques such as intramuscular, subcutaneous, and/or intradermal injection of (i) one or more HDAC11 inhibitors or a composition comprising one or more HDAC11 inhibitors and/or (ii) HDAC11-activated M2 macrophages or a composition comprising HDAC11-activated M2 macrophages.

In an aspect, local administration of (i) one or more HDAC11 inhibitors or a composition comprising a therapeutically effective amount of one or more HDAC11 inhibitors and/or (ii) HDAC11-activated M2 macrophages or a composition comprising HDAC11-activated M2 macrophages can comprise delivery to one or more body systems in need thereof. In an aspect, local administration or local delivery of (i) one or more HDAC11 inhibitors or a composition comprising one or more HDAC11 inhibitors and/or (ii) HDAC11-activated M2 macrophages or a composition comprising HDAC11-activated M2 macrophages can comprise a transdermal administration, administration via implant, administration via suppository, implantation via a biodegradable polymer or matrix, implantation via a biodegradable hydrogel, or any combination thereof.

In an aspect of a disclosed method of treating a subject, administering (i) one or more HDAC11 inhibitors or a composition comprising one or more HDAC11 inhibitors and/or r (ii) HDAC11-activated M2 macrophages or a composition comprising HDAC11-activated M2 macrophages can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, (i) one or more HDAC11 inhibitors or a composition comprising one or more HDAC11 inhibitors and/or (ii) HDAC11-activated M2 macrophages or a composition comprising HDAC11-activated M2 macrophages can be administered via multiple routes of administration. In an aspect of a disclosed method of treating a subject, following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, inflammation in one or more of the subject's body systems can be diminished and/or decreased. In an aspect of a disclosed method of treating a subject, the decrease in inflammation in one or more of the subject's body systems can be measured objectively and/or subjectively.

In an aspect of a disclosed method of treating a subject, following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, the pathology of the subject's disease or disorder can be diminished and/or decreased. In an aspect of a disclosed method of treating a subject, wherein, following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, the subject's symptoms can be diminished and/or decreased.

In an aspect of a disclosed method of treating a subject, wherein, following administration of (i) one or more HDAC11 inhibitors or a composition comprising one or more HDAC11 inhibitors or (ii) HDAC11-activated M2 macrophages or a composition comprising HDAC11-activated M2 macrophages to the subject, the subject's quality of life can be improved and/or enhanced. In an aspect, quality of life can be measured objectivity and/or subjectively.

In an aspect of a disclosed method of treating a subject, wherein, following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, the one or more transplanted organs can be not rejected.

In an aspect of a disclosed method of treating a subject, wherein, following administration of (i) one or more HDAC11 inhibitors or a composition comprising one or more HDAC11 inhibitors or (ii) HDAC11-activated M2 macrophages or a composition comprising HDAC11-activated M2 macrophages to the subject, the one or more transplanted organs can be spared from rejection.

In an aspect of a disclosed method of treating a subject, wherein, following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, the healing of the subject's one or more chronic wounds can be improved and/or enhanced. In an aspect of a disclosed method of treating a subject, wherein improving and/or enhancing healing of one or more chronic wounds can comprise improving angiogenesis, innervation, cellular migration, or any combination thereof. In an aspect of a disclosed method of treating a subject, wherein following administration of (i) one or more HDAC11 inhibitors or a composition comprising one or more HDAC11 inhibitors and/or (ii) HDAC11-activated M2 macrophages or a composition comprising HDAC11-activated M2 macrophages to the subject, one or more of the subject's body systems can experience and/or show signs of cellular regeneration and/or cellular repair.

In an aspect of a disclosed method of treating a subject, wherein the one or more body systems experiencing and/or showing signs of regeneration and/repair can comprise the cardiovascular system. In an aspect of a disclosed method of treating a subject, wherein previously ischemic and/or infarcted and/or languishing areas of the cardiovascular system can show signs of regeneration and/or repair. In an aspect of a disclosed method of treating a subject, the one or more body systems experiencing and/or showing signs of regeneration and/repair can comprise the central nervous system and/or peripheral nervous system. In an aspect of a disclosed method of treating a subject, wherein previously ischemic and/or infarcted/or languishing areas of the central nervous system and/or peripheral nervous system can show signs of regeneration and/or repair. In an aspect of a disclosed method of treating a subject, wherein the one or more body systems experiencing and/or showing signs of regeneration and/repair can comprise the integumentary system. In an aspect of a disclosed method of treating a subject, wherein the one or more body systems experiencing and/or showing signs of regeneration and/repair can comprise the digestive system. In an aspect, a disclosed method of treating a subject can further comprise diminishing and/or decreasing inflammation in one or more of the subject's body systems. Body systems are disclosed supra.

In an aspect, a decrease in inflammation in one or more of the subject's body systems can be measured objectively and/or subjectively. In an aspect, a disclosed method of treating a subject can further comprise diminishing and/or decreasing the pathology of the subject's disease or disorder. In an aspect, a disclosed method of treating a subject can further comprise diminishing and/or decreasing the subject's symptoms. In an aspect, a disclosed method of treating a subject can further comprise improving and/or enhancing the subject's quality of life. In an aspect, quality of life can be measured objectivity and/or subjectively. In an aspect, a disclosed method of treating a subject can further comprise preventing the rejection of one or more transplanted organs in the subject. In an aspect, a disclosed method of treating a subject can further comprise minimizing the risk of rejection of one or more transplanted organs in the subject. In an aspect, a disclosed method of treating a subject can further comprise improving and/or enhancing the healing of one or more chronic wounds in the subject. In an aspect, a disclosed method of treating a subject can further comprise improving and/or enhancing healing of one or more chronic wounds. In an aspect, a disclosed method of treating a subject can further comprise improving and/or enhancing angiogenesis, innervation, cellular migration, or any combination thereof in and/or around one or more chronic wounds in the subject.

In an aspect, a disclosed method of treating a subject can further comprise improving and/or enhancing cellular regeneration and/or cellular repair or one or more of the subject's affected body systems. In an aspect, a disclosed method of treating a subject can further comprise improving and/or enhancing cellular regeneration and/or cellular repair in the subject's central nervous system and/or peripheral nervous system. In an aspect, a disclosed method of treating a subject can further comprise improving and/or enhancing cellular regeneration and/or cellular repair in the subject's integumentary system. In an aspect, a disclosed method of treating a subject can further comprise improving and/or enhancing cellular regeneration and/or cellular repair in the subject's digestive system.

In an aspect, a disclosed method of treating a subject can further comprise repeating one or more times one or more steps of a disclosed method. In an aspect of a disclosed method of treating a subject, a disclosed obtaining step can be repeated, a disclosed isolating step can be repeated, a disclosed contacting step can be repeated, a disclosed activating step can be repeated, a disclosed polarizing step can be repeated, a disclosed incubating step can be repeated, a disclosed administering step can be repeated, or any combination thereof. In an aspect, a disclosed method of treating a subject can further comprise repeating an obtaining step, repeating an isolating step, repeating a contacting step, repeating an activating step, repeating a polarizing step, repeating an incubating step, repeating an administering step, or repeating one or more time any combination of steps thereof.

In an aspect of a disclosed method of treating a subject, a disclosed improvement and/or enhancement (such as, for example, in the subject's quality of life and/or movement and/or would healing and/or angiogenesis, etc.) can comprise a 10%, 20%, 30%, 40%, 50%, 60%, 70%, 80%, 90%, 100%, or any amount of improvement and/or enhancement when compared to a control subject (such as, for example, a subject that has not received (i) one or more HDAC11 inhibitors or a composition comprising one or more HDAC11 inhibitors and/or (ii) HDAC11-activated M2 macrophages or a composition comprising HDAC11-activated M2 macrophages). In an aspect, a disclosed improvement and/or enhancement (such as, for example, in the subject's quality of life and/or movement and/or would healing and/or angiogenesis, etc.) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not received (i) one or more HDAC11 inhibitors or a composition comprising one or more HDAC11 inhibitors and/or (ii) HDAC11-activated M2 macrophages or a composition comprising HDAC11-activated M2 macrophages).

In an aspect of a disclosed method of treating a subject, a disclosed diminishment (such as, for example, in the subject's pain and/or wound size and/or restrictions) can comprise a 10%, 20%, 30%, 40%, 50%, 60%, 70%, 80%, 90%, 100%, or any amount of a decrease when compared to a control subject (such as, for example, a subject that has not received (i) one or more HDAC11 inhibitors or a composition comprising one or more HDAC11 inhibitors and/or (ii) HDAC11-activated M2 macrophages or a composition comprising HDAC11-activated M2 macrophages). In an aspect, a disclosed diminishment (such as, for example, in the subject's pain and/or wound size and/or restrictions) can comprise a 10-20%, 20-30%, 30-40%, 40-50%, 50-60%, 60-70%, 70-80%, 80-90%, or 90-100% or any amount of a decrease when compared to a control subject (such as a subject that has not received (i) one or more HDAC11 inhibitors or a composition comprising one or more HDAC11 inhibitors and/or (ii) HDAC11-activated M2 macrophages or a composition comprising HDAC11-activated M2 macrophages).

In an aspect, a disclosed method of treating a subject can further comprise administering to a disclosed subject one or more therapeutic agents and/or active agents. In an aspect, one or more disclosed therapeutic agents and/or active agents can comprise an immune modulator. In an aspect, a disclosed immune modulator can refer to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions.

In an aspect of a disclosed method of treating a subject, administering can comprise intravenous administration of one or more therapeutic agents and/or active agents. In an aspect, administering can comprise systemic administration or local administration of one or more therapeutic agents and/or active agents.

In an aspect of a disclosed method of treating a subject, local delivery or local administration can refer to delivering or administering one or more therapeutic agents and/or active agents directly to a target site within a subject. For example, the one or more therapeutic agents and/or active agents can be locally delivered by direct injection into an affected body system (such as one, for example, having inflammation). Local delivery or local administration can also include topical applications or localized injection techniques such as intramuscular, subcutaneous, and/or intradermal injection of one or more therapeutic agents and/or active agents. In an aspect, local administration of one or more therapeutic agents and/or active agents can comprise delivery to one or more body systems in need thereof. In an aspect, local administration or local delivery of one or more therapeutic agents and/or active agents can comprise a transdermal administration, administration via implant, administration via suppository, implantation via a biodegradable polymer or matrix, implantation via a biodegradable hydrogel, or any combination thereof. A skilled clinician can determine the best route of administration for a subject at a given time.

In an aspect of a disclosed method of treating a subject, one or more HDAC11 inhibitors or a composition comprising one or more HDAC11 inhibitors can be administered to the subject prior to, concurrent with, or after administration of one or more therapeutic agents and/or active agents. In an aspect of a disclosed method of treating a subject, one or more HDAC11 inhibitors or a composition comprising one or more HDAC11 inhibitors can be administered one or more times and the one or more therapeutic agents and/or active agents can be administered one or more times. In an aspect of a disclosed method of treating a subject, one or more HDAC11 inhibitors or a composition comprising one or more HDAC11 inhibitors can be administered one time and the one or more therapeutic agents and/or active agents can be administered one or more times. Dosing schedules can be determined by skilled person in the art. In an aspect of a disclosed method of treating a subject, the subject's physiology and/or functionality can be restored to normal or near normal levels (e.g., with little or no inflammation or little or no signs and/or symptoms of inflammation). In an aspect of a disclosed method of treating a subject, the disclosed subject's behavior and/or physiology can be modulated.

In an aspect, a therapeutically effective amount of HDAC11-activated M2 macrophages or a composition comprising HDAC11-activated M2 macrophages can comprise a range of macrophages activated by from about 50 nM to about 25 μM of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, a disclosed method of treating a subject can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method of treating a subject can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect of a disclosed method of treating a subject, modifying the treating step can comprise changing the amount of (i) one or more HDAC11 inhibitors or a composition comprising one or more HDAC11 inhibitors and/or (ii) HDAC11-activated M2 macrophages or a composition comprising HDAC11-activated M2 macrophages administered to the subject, changing the frequency of administration of (i) one or more HDAC11 inhibitors or a composition comprising one or more HDAC11 inhibitors and/or (ii) HDAC11-activated M2 macrophages or a composition comprising HDAC11-activated M2 macrophages, changing the duration of administration of (i) one or more HDAC11 inhibitors or a composition comprising one or more HDAC11 inhibitors and/or (ii) HDAC11-activated M2 macrophages or a composition comprising HDAC11-activated M2 macrophages, changing the route of administration of (i) one or more HDAC11 inhibitors or a composition comprising one or more HDAC11 inhibitors and/or (ii) HDAC11-activated M2 macrophages or a composition comprising HDAC11-activated M2 macrophages, or any combination thereof. In an aspect of a disclosed method of treating a subject, modifying the administering step can comprise changing the amount of (i) one or more HDAC11 inhibitors or a composition comprising one or more HDAC11 inhibitors and/or (ii) HDAC11-activated M2 macrophages or a composition comprising HDAC11-activated M2 macrophages administered to the subject, changing the frequency of administration of (i) one or more HDAC11 inhibitors or a composition comprising one or more HDAC11 inhibitors and/or (ii) HDAC11-activated M2 macrophages or a composition comprising HDAC11-activated M2 macrophages, changing the duration of administration of (i) one or more HDAC11 inhibitors or a composition comprising one or more HDAC11 inhibitors and/or (ii) HDAC11-activated M2 macrophages or a composition comprising HDAC11-activated M2 macrophages, changing the route of administration of (i) one or more HDAC11 inhibitors or a composition comprising one or more HDAC11 inhibitors and/or (ii) HDAC11-activated M2 macrophages or a composition comprising HDAC11-activated M2 macrophages, or any combination thereof.

In an aspect, a disclosed method of treating a subject can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method of treating a subject, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer (i) one or more HDAC11 inhibitors or a composition comprising one or more HDAC11 inhibitors and/or (ii) HDAC11-activated M2 macrophages or a composition comprising HDAC11-activated M2 macrophages. In an aspect, metabolic and/or physiologic data can inform the clinician when make subsequent treatment decisions.

### 4. Methods of Providing Adaptive Cell Therapy

Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages. Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, wherein following the administering step, inflammation in one or more of the subject's body systems is decreased and/or diminished.

Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, thereby decreasing and/or diminishing inflammation in one or more of the subject's body systems. Disclosed herein is a method of providing adaptive cell therapy to a subject, the method comprising administering to a subject in need thereof a therapeutically effective amount of HDAC11-activated M2 macrophages or a composition comprising HDAC11-activated M2 macrophages, and decreasing and/or diminishing inflammation in one or more of the subject's body systems.

In an aspect, a disclosed method of providing adaptive cell therapy to a subject can further comprise generating HDAC11-activated and M2 polarized macrophages. In an aspect, generating HDAC11-activated and M2 polarized macrophages can be an ex vivo process. In an aspect, HDAC11-activated macrophages can be any disclosed HDAC11-activated and M2 polarized macrophages. In an aspect, a disclosed method of providing adaptive cell therapy to a subject can further comprise activating M0 macrophages or naïve macrophages.

In an aspect, activating M0 macrophages or naïve macrophages can comprise contacting a population of naïve macrophages with one or more HDAC11 inhibitors. In an aspect of a disclosed method of providing adaptive cell therapy to a subject, following the contacting of the M0 macrophages or naïve macrophages with HDAC11 inhibitor, the HDAC11-activated macrophages are then polarized towards an anti-inflammatory phenotype. In an aspect of a disclosed method of providing adaptive cell therapy to a subject, following the contacting of the M0 macrophages or naïve macrophages with HDAC11 inhibitor, the HDAC11-activated macrophages are then polarized towards a M2 phenotype.

In an aspect, a disclosed method of providing adaptive cell therapy to a subject can further comprise contacting a population of polarized macrophages with an HDAC11 inhibitor. In an aspect of a disclosed method of providing adaptive cell therapy to a subject following contacting a population of polarized macrophages with an HDAC11 inhibitor, the anti-inflammatory phenotype in enhanced and/or improved. In an aspect of a disclosed method of providing adaptive cell therapy to a subject, following contacting a population of polarized macrophages with an HDAC11 inhibitor, wherein the macrophages are polarized towards an enhanced and/or improved M2 phenotype. In an aspect of a disclosed method of providing adaptive cell therapy to a subject, contacting a population of M0 or naïve macrophages with one or more HDAC11 inhibitors can enhance and/or improve the M2 phenotype of the macrophages upon polarization. In an aspect of a disclosed method of providing adaptive cell therapy to a subject, contacting a population of polarized macrophages with one or more HDAC11 inhibitors can enhance and/or improve the M2 phenotype of the polarized macrophages.

In an aspect of a disclosed method of providing adaptive cell therapy to a subject, contacting a population of M0 or naïve macrophages with one or more HDAC11 inhibitors can enhance and/or improve the anti-inflammatory phenotype of the macrophages upon polarization. In an aspect of a disclosed method of providing adaptive cell therapy to a subject, contacting a population of polarized macrophages with one or more HDAC11 inhibitors can enhance and/or improve the anti-inflammatory phenotype of the polarized macrophages.

In an aspect, HDAC11-activated macrophages or HDAC11 polarized macrophage can refer to a M0 or naïve macrophage that has been treated ex vivo with an HDAC11 inhibitor (such as, for example a disclosed HDAC11 inhibitor or disclosed selective HDAC11 inhibitor). In an aspect, disclosed HDAC11-activated macrophages are first treated ex vivo with a disclosed HDAC11 inhibitor and then treated ex vivo with a disclosed macrophage polarizing agent. In an aspect, disclosed HDAC11 polarized macrophages are first treated ex vivo with a macrophage polarizing agent and then treated ex vivo with a disclosed HDAC11 inhibitor.

In an aspect of a disclosed method of providing adaptive cell therapy to a subject, wherein contacting a population of naïve macrophages with an HDAC11 inhibitor comprises a pre-determined amount of time. For example, in an aspect, contacting a population of naïve macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of naïve macrophages with an HDAC11 inhibitor can comprise more than 24 hours.

In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, contacting a population of polarized macrophages with an HDAC11 inhibitor can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of polarized macrophages with an HDAC11 inhibitor can comprise more than 24 hours.

In an aspect of a disclosed method, contacting a population of naïve macrophages with an HDAC11 inhibitor can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between.

In an aspect, naïve macrophages can be contacted with an HDAC11 inhibitor one or more times, and then polarized to M2 macrophages. In an aspect, polarized macrophages can be contacted with an HDAC11 inhibitor one or more times.

In an aspect of a disclosed method of providing adaptive cell therapy to a subject, a disclosed subject in need thereof can have one or more chronic wounds. In an aspect, a disclosed chronic wound can comprise infected and/or traumatic wounds, arterial ulcer, venous ulcers, pressure ulcers, diabetic ulcers, ischemic ulcers, or any combination thereof. In an aspect, a disclosed subject can have one or more foot ulcers. In an aspect, a disclosed subject can have diabetes. In an aspect, a disclosed subject can be the recipient of one or more solid organ transplants. In an aspect, a disclosed subject can have irritable bowel disease (IBD). In an aspect, a disclosed subject can have one or more inflammatory diseases. In an aspect, a disclosed subject can have one or more skin inflammatory diseases. In an aspect, a disclosed subject can have suffered one or more burns. In an aspect, a disclosed subject can have incurred an ischemic injury. In an aspect, a disclosed ischemic injury can affect the subject's brain and/or the subject's heart. In an aspect, a disclosed subject can have suffered one or more myocardial infarctions. In an aspect, a disclosed subject can have suffered one or more transient ischemic attacks.

In an aspect, a disclosed subject can have an autoinflammatory disease (AIF). In an aspect, a disclosed autoinflammatory disease can comprise familial Mediterranean fever (FMF), hyper IgD syndrome (HIDS), tumor necrosis factor receptor associated autoinflammatory syndrome (TRAPS), cryopyrin associated periodic syndromes (CAPS), or Blau syndrome and pyogenic sterile arthritis pyoderma gangrenosum and acne syndrome (PAPA).

In an aspect of a disclosed method of providing adaptive cell therapy to a subject, a disclosed subject can have an autoimmune disease (AD). In an aspect, a disclosed autoimmune disease or disorder can comprise *Acromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, *Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis acute disseminated encephalomyelitis (ADEM), Autoimmune gastritis, Autoimmune hemolytic anemia (AIHA), Autoimmune hepatitis (AIH), *Autoimmune hyperlipidemia, *Autoimmune hypophysitis, *Autoimmune inner ear disease (AIED), *Autoimmune lymphoproliferative syndrome (ALPS), *Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, *Autoimmune retinopathy (AIR), *Autoimmune sudden sensorineural hearing loss (SNHL), Balo disease, Behçet's disease, *Birdshot chorioretinopathy/birdshot uveitis, Bullous pemphigoid, *Castleman disease, Celiac disease, *Chagas disease, Chronic inflammatory demyelinating polyneuropathy (CIDP), *Chronic urticaria (CU), Churg-Strauss syndrome/cosinophilic granulomatosis with polyangiitis (EGPA), Cogan's syndrome, Cold agglutinin disease, CREST syndrome|limited cutaneous systemic sclerosis, Crohn's disease (CD), *Cronkhite-Canada syndrome (CSS), *Cryptogenic organizing pneumonia (COP), Dermatitis herpetiformis, Dermatomyositis, Devic's disease/neuromyelitis optica (NMO), Diabetes, type 1, Discoid lupus, *Dressler's syndrome/postmyocardial infarction/postpericardiotomy syndrome, *Eczema/Atopic Dermatitis, *Endometriosis, *Eosinophilic esophagitis, Eosinophilic fasciitis, *Erythema nodosum, *Essential mixed cryoglobulinemia, Evans syndrome, *Fibrosing alveolitis/Idiopathic pulmonary fibrosis (IPF), *Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, *Hidradenitis suppurativa, Hurst's disease/acute hemorrhagic leukoencephalitis (AHLE), *Hypogammaglobulinemia, IgA nephropathy/Berger's disease, Immune-mediated/autoimmune necrotizing myopathy (IMNM), Immune thrombocytopenia (ITP) thrombocytopenia purpura/autoimmune thrombocytopenia, *Inclusion body myositis, *IgG4-related sclerosing disease (ISD), *Interstitial cystitis, Juvenile idiopathic arthritis/Adult-onset Still's disease, Juvenile polymyositis|Juvenile dermatomyositis|juvenile myositis, *Kawasaki disease, Lambert-Eaton myasthenic syndrome (LEMS), *Leukocytoclastic vasculitis, *Lichen planus, *Lichen sclerosus, *Ligneous conjunctivitis, Linear IgA disease (LAD)|linear IgA bullous dermatosis (LABD), Lupus nephritis, *Lyme disease/chronic Lyme disease/post-treatment Lyme disease syndrome (PTLDS), *Lymphocytic colitis/microscopic colitis, Ménière's disease, *Microscopic polyangiitis (MPA)/ANCA-associated vasculitis, Mixed connective tissue disease (MCTD), *Mooren's ulcer, *Mucha-Habermann disease, *Multifocal motor neuropathy, Multiple sclerosis (MS), *Myalgic encephalomyelitis (ME)/Chronic fatigue syndrome (CFS), Myasthenia gravis (MG), *Narcolepsy, Ocular cicatricial pemphigoid, *Opsoclonus-myoclonus syndrome (OMS), Palindromic rheumatism, Parancoplastic cerebellar degeneration, Parancoplastic pemphigus, *Parry-Romberg syndrome (PRS)/Hemifacial atrophy (HFA)/Progressive facial hemiatrophy, Paroxysmal nocturnal hemoglobinuria (PNH), *Peripheral uveitis/pars planitis, *PANS/PANDAS, Parsonage-Turner syndrome, Pemphigus gestationis/herpes gestationis, Pemphigus foliaceus, Pemphigus vulgaris, *Pernicious anemia, POEMS syndrome, Polyarteritis nodosa, Polymyalgia rheumatica, Polymyositis, *Postural orthostatic tachycardia syndrome (POTS), Primary biliary cirrhosis (PBC)/primary biliary cholangitis, *Primary sclerosing cholangitis (PSC), Psoriasis, Palmoplantar Pustulosis, Psoriatic arthritis, *Pulmonary fibrosis, idiopathic (IPF), Pure red cell aplasia (PRCA), *Pyoderma gangrenosum, Raynaud's syndrome/phenomenon, Reactive arthritis/Reiter's syndrome, *Reflex sympathetic dystrophy syndrome (RSD)/Complex regional pain syndrome (CRPS), Relapsing polychondritis, *Restless leg syndrome (RLS)/Willis-Ekbom disease, Rheumatic fever, Rheumatoid arthritis, Sarcoidosis, Schmidt syndrome/autoimmune polyendocrine syndrome type II, *Scleritis, Scleroderma, *Serpiginous choroidopathy, Sjögren's syndrome, *Stiff person syndrome (SPS), Small fiber sensory neuropathy, Systemic lupus erythematosus (SLE), *Subacute bacterial endocarditis (SBE), *Susac syndrome, *Sydenham's chorea, *Sympathetic ophthalmia, *Takayasu's arteritis (vasculitis), Testicular autoimmunity (vasculitis, orchitis), *Tolosa-Hunt syndrome, *Transverse myelitis (TM), *Tubulointerstitial nephritis uveitis syndrome (TINU), Ulcerative colitis (UC), Undifferentiated connective tissue disease (UCTD), *Uveitis|anterior/intermediate/posterior, *Vasculitis, Vitiligo, *Vogt-Koyanagi-Harada syndrome (VKH), or any combination thereof.

In an aspect of a disclosed method of providing adaptive cell therapy to a subject, a disclosed subject can be male or female. In an aspect, a subject can be an adult, a teenager, an adolescent, a child, a toddler, a baby, or an infant. In an aspect, a subject can have received treatment for one or more disclosed AIFs or ADs. In an aspect of a disclosed method of providing adaptive cell therapy to a subject, a subject can be treatment-naïve.

In an aspect, a disclosed method of providing adaptive cell therapy to a subject can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval.

In an aspect, a blood sample can be collected from a subject prior to treatment, during treatment, after treatment, or any combination thereof. In an aspect, a blood sample can be collected from a subject at any time deemed medically and/or clinically appropriate by the skilled clinician. In an aspect, a disclosed method of providing adaptive cell therapy to a subject can further comprise isolating monocytes from peripheral blood monocular cells in the subject's blood sample. In an aspect, a disclosed method of providing adaptive cell therapy to a subject can further comprise isolating bone marrow derived monocytes from the subject's blood sample. In an aspect, a disclosed method of providing adaptive cell therapy to a subject can further comprise isolating monocytes from the subject's blood sample. In an aspect, a disclosed method of providing adaptive cell therapy to a subject can further comprise isolating naïve macrophages (M0) from the subject's blood sample.

In an aspect, a disclosed method of providing adaptive cell therapy to a subject can further comprise subjecting a disclosed blood sample to centrifugation. In an aspect, a disclosed method can further comprise separating the blood sample into its component parts using, for example, centrifugation, apheresis, or any technique to the skilled person. In an aspect, a disclosed separating step can comprise generating a layer of clear fluid, a layer of red fluid, and a thin layer in between the clear fluid layer and the red fluid layer. In an aspect, a disclosed red layer can comprise red blood cells. In an aspect, a disclosed clear layer can comprise plasma. In an aspect, a disclosed thin layer in between the red layer and the clear layer can comprise the buffy coat. In an aspect, a disclosed buffy coat can comprise white blood cells and platelets. In an aspect, a disclosed method can further comprise isolating peripheral blood mononuclear cells (PMBCs) from the buffy coat. In an aspect, PMBCs can comprise lymphocytes and/or monocytes. In an aspect, macrophages can be derived from monocytes. In an aspect, isolating monocytes can be done by any method and/or technique known to the skilled person. For example, in an aspect, a conical blood filter and the buffy coat having approximately 10 mL and 70 mL respectively, can be used. In an aspect, a disclosed sample can be collected in a tube, e.g., a 50 mL tube. In an aspect, if using a conical blood filter, then dilute the sample by adding PBS, for example, at room temperature. In an aspect, a disclosed method can add 10 mL of Ficoll-Hypaque-1077 (or Lymphoprep) to a disclosed centrifuge tube at room temperature. In an aspect, a disclosed method can comprise carefully layering the blood (e.g., 3 mL) onto the Ficoll-Hypaque-1077. In an aspect, blood can stay in the upper layer and the Ficoll-Hypaque 1077 in the lowest one. In an aspect, a disclosed method can comprise centrifuging at 400×g for about 30 min at room temperature. In an aspect of a disclosed method, the centrifuge can be set for 0 (zero) acceleration and 0 (zero) break. In an aspect, after centrifugation, 3 layers can be visible. In an aspect, an upper layer can comprise plasma and the Ficoll-Hypaque. In an aspect, an opaque interface can comprise the mononuclear cells. In an aspect, a lower lever can comprise the red blood cells. In an aspect of a disclosed method, a Pasteur pipette can be used to aspirate the upper layer until close to the opaque phase comprising the mononuclear cells. In an aspect, the upper layer can be discarded. In an aspect of a disclosed method, the disclosed opaque interface can be transferred to a tube (e.g., 50 mL). In an aspect, a tube can be filled with cold PBS. In an aspect of a disclosed method can comprise centrifuged at 250×g for about 10 min at 4° C. In an aspect, a disclosed centrifuge can be at full acceleration and break 5. In an aspect of the disclosed method, at the end of the first washing, the supernatant can appear turbid due to presence of platelets. In an aspect of a disclosed method, the supernatant can be aspirated and the pellet can be resuspended with cold PBS. In an aspect of a disclosed method, centrifuging can be at 250×g for about 10 min at 4° C. In an aspect of a disclosed method, washing steps can be repeated 3-4 times or can be repeated until a clear supernatant is obtained. In an aspect of a disclosed method, monocytes can be isolated by negative selection and/or cell adhesion. Both techniques are known to the skilled person in the art. In an aspect, a disclosed method of providing adaptive cell therapy to a subject can further comprise isolating resting or M0 macrophages from the buffy coat.

In an aspect, a disclosed method of providing adaptive cell therapy to a subject can further comprise differentiating monocytes into resting or M0 macrophages. In an aspect of a disclosed method of providing adaptive cell therapy to a subject, disclosed macrophages can be resting or M0 macrophages. In an aspect, a disclosed method of providing adaptive cell therapy to a subject can further comprise polarizing the resting or M0 macrophages into a M2 phenotype or anti-inflammatory phenotype. In an aspect, a disclosed method of providing adaptive cell therapy to a subject can further comprise polarizing the resting or M0 macrophages into an alternatively activated macrophage phenotype. In an aspect, a disclosed method of providing adaptive cell therapy to a subject can further comprise polarizing the resting or M0 macrophages into a M2 phenotype or anti-inflammatory phenotype comprises contacting the resting or M0 macrophages with IL-4, IL-10, IL-13, TGFβ-1, PGE2, or any combination thereof. In an aspect, a disclosed method of providing adaptive cell therapy to a subject can further comprise, resting or M0 macrophages can be polarized into a M2 phenotype or anti-inflammatory phenotype.

In an aspect, a disclosed method of providing adaptive cell therapy to a subject can further comprise, resting or M0 macrophages can be polarized into an alternatively activated macrophage phenotype. In an aspect, a disclosed method of providing adaptive cell therapy to a subject can further comprise, resting or M0 macrophages can be polarized into a M2 phenotype or anti-inflammatory phenotype comprises contacting the resting or M0 macrophages with IL-4, IL-10, IL-13, TGFβ-1, PGE2, or any combination thereof.

In an aspect, a disclosed method of providing adaptive cell therapy to a subject can further comprise, polarizing resting or M0 macrophages into a M2 phenotype or an anti-inflammatory phenotype can comprise about a 1-day to about a 10-day incubation. In an aspect, a disclosed method of providing adaptive cell therapy to a subject can further comprise, polarizing resting or M0 macrophages into a M2 phenotype or an anti-inflammatory phenotype can comprise about a 3-day to about a 7-day incubation. In an aspect, a disclosed method of providing adaptive cell therapy to a subject can further comprise, polarizing the resting or M0 macrophages into a M2 phenotype or an anti-inflammatory phenotype can comprise about a 1-day incubation, about a 2-day incubation, about a 3-day incubation, about a 4-day incubation, about a 5-day incubation, about a 6-day incubation, about a 7-day incubation, about a 8-day incubation, about a 9-day incubation, about a 10-day incubation, or more than a 10-day incubation. In an aspect, a disclosed method of providing adaptive cell therapy to a subject can further comprise, wherein following the contacting of the resting or M0 macrophages with a disclosed HDAC11 inhibitor, polarizing macrophages can be incubated from about 1 day to about 10 days, or from about 3 days to about 7 days, or for about 1 day, about 2 days, about 3 days, about 4 days, about 5 days, about 6 days, about 7 days, about 8 days, about 9 days, about 10 days, or more than 10 days.

In an aspect, one or more steps of a disclosed method of providing adaptive cell therapy to a subject can be performed ex vivo. In an aspect, a therapeutically effective amount of an HDAC11 inhibitor can comprise from about 50 nM to about 25 μM of an HDAC11-inhibitor. In an aspect, a therapeutically effective amount of an HDAC11-inhibitor can comprise an amount that can elicits and/or can provoke one or more measurable anti-inflammatory responses.

In an aspect, a disclosed method of providing adaptive cell therapy can comprise one or more disclosed HDAC11 inhibitors or a composition comprising one or more disclosed HDAC11 inhibitors. In an aspect, the one or more disclosed HDAC11 inhibitors or a composition comprising one or more disclosed HDAC11 inhibitors can be administered one or more times.

In an aspect of a disclosed method of providing adaptive cell therapy to a subject, a disclosed HDAC11 inhibitor can comprise elevenostat (JB3-22), garcinol, SIS7, SIS17, FT895, romidepsin, quisinostat, or any combination thereof. In an aspect of a disclosed method of providing adaptive cell therapy to a subject, a disclosed HDAC11 inhibitor can comprise any commercially available HDAC11 inhibitor or a combination of commercially available HDAC11 inhibitors. In an aspect, an HDAC11 inhibitor can comprise a selective HDAC11 inhibitor or HDAC11 selective inhibitor and can refer to a compound that preferentially inhibits histone deacetylase 11 over one or more other histone deacetylase isoforms, e.g., HDAC1, HDAC2, HDAC3, HDAC4, HDAC5, HDAC6, HDAC7, HDAC8, HDAC9, and/or HDAC10 in a cell-based in vitro assay. For example, a compound having a HDAC11 IC_{SO}=5 nM and a HDAC1 IC_{SO} of 500 nM is a selective HDAC11 inhibitor that is 100-fold more selective over HDAC1; a compound having a HDAC11 IC_{SO}=5 nM, a HDAC1 IC_{SO}=500 nM, and a HDAC3 IC_{SO}=50 nM is a selective HDAC11 inhibitor that is 100-fold more selective over HDAC1 and 10-fold more selective over HDAC3; and so on. In one aspect, a selective HDAC11 inhibitor can preferentially inhibit HDAC11 over HDAC1, HDAC2, HDAC3, HDAC4, HDAC5, HDAC6, HDAC7, HDAC8, HDAC9, and/or HDAC10.

In an aspect, the selective HDAC11 inhibitor can preferentially inhibit HDAC11 over one or more other HDAC isoforms. In an aspect, the selective HDAC11 inhibitor can be at least about 5-fold more selective over one or more other HDAC isoforms. In an aspect, the selective HDAC11 inhibitor can be at least about 10-fold more selective over one or more other HDAC isoforms. In an aspect, the selective HDAC11 inhibitor can be at least about 15-fold more selective over one or more other HDAC isoforms. In aspect, the selective HDAC11 inhibitor can be at least about 20-fold more selective over one or more other HDAC isoforms. In an aspect, the selective HDAC11 inhibitor can be at least about 30-fold more selective over one or more other HDAC isoforms. In an aspect, the selective HDAC11 inhibitor can be at least about 40-fold more selective over one or more other HDAC isoforms. In an aspect, the selective HDAC11 inhibitor can be at least about 50-fold more selective over one or more other HDAC isoforms. In an aspect, the selective HDAC6 inhibitor can be at least about 100-fold more selective over one or more other HDAC isoforms. In another aspect, the selective HDAC11 inhibitor can be at least about 150-fold more selective over one or more other HDAC isoforms. In an aspect, the selective HDAC11 inhibitor can be at least about 200-fold more selective over one or more other HDAC isoforms. In an aspect, the selective HDAC11 inhibitor can be at least about 250-fold more selective over one or more other HDAC isoforms. In an aspect, the selective HDAC11 inhibitor can be at least about 500-fold more selective over one or more other HDAC isoforms. In an aspect, the selective HDAC11 inhibitor can be at least about 750-fold more selective over one or more other HDAC isoforms. In an aspect, the selective HDAC11 inhibitor can be at least about 1000-fold more selective over one or more other HDAC isoforms. In an aspect, the selective HDAC11 inhibitor can be at least about 2000-fold more selective over one or more other HDAC isoforms. In an aspect, the selective HDAC11 inhibitor can be at least about 3000-fold more selective over one or more other HDAC isoforms. HDAC11 selectivity over the other HDAC isoforms in cell-based assays can be determined using methods known in the art. In another aspect, the selective HDAC11 inhibitor can be at about 10-fold to about 3000-fold more selective over one or more other HDAC isoforms. In an aspect, the selective HDAC11 inhibitor can be at about 20-fold to about 3000-fold more selective over one or more other HDAC isoforms. In an aspect, the selective HDAC11 inhibitor can be at about 50-fold to about 3000-fold more selective over one or more other HDAC isoforms. In an aspect, the selective HDAC11 inhibitor can be at about 100-fold to about 3000-fold more selective over one or more other HDAC isoforms. In an aspect, the selective HDAC11 inhibitor can be at about 500-fold to about 3000-fold more selective over one or more other HDAC isoforms.

In an aspect of a disclosed method, about 50 nM to about 25 μM of a disclosed HDAC11 inhibitor can polarize resting or M0 macrophages into a M2 phenotype or an anti-inflammatory phenotype.

In an aspect of a disclosed method of providing adaptive cell therapy to a subject, contacting the resting or M0 macrophages with one or more HDAC11 inhibitors can occur prior to polarizing the resting or M0 macrophages into the M2 phenotype or anti-inflammatory phenotype. In an aspect of a disclosed method of providing adaptive cell therapy to a subject, contacting the resting or M0 macrophages with one or more HDAC11 inhibitors can occur after polarizing the resting or M0 macrophages into the M2 phenotype or anti-inflammatory phenotype. In an aspect of a disclosed method of providing adaptive cell therapy to a subject, contacting the resting or M0 macrophages with one or more HDAC11 inhibitors can occur during the polarizing the resting or M0 macrophages into the M2 phenotype or anti-inflammatory phenotype.

In an aspect, a disclosed method of providing adaptive cell therapy to a subject can further comprise confirming the resulting macrophages demonstrate the M2 phenotype or anti-inflammatory phenotype. In an aspect, confirming the M2 phenotype or anti-inflammatory phenotype can comprise detecting a change in the expression level of one or more relevant genes and/or relevant proteins. In an aspect of a disclosed method of providing adaptive cell therapy to a subject, detecting a change in the expression level can comprise detecting an increase in the expression level of one or more relevant genes and/or relevant proteins. In an aspect, detecting a change in expression level can comprise detecting a decrease in the expression level of one or more relevant genes and/or relevant proteins. In an aspect of a disclosed method of providing adaptive cell therapy to a subject, one or more disclosed relevant genes and/or relevant proteins can comprise IL-10, TGF-β, Fizz1, Arg1, Mrc1 (CD206), or any combination thereof. In an aspect, detecting a change in the expression level can comprise detecting an increase in the expression level of IL-10, TGF-β, Fizz1, Arg1, Mrc1 (CD206), or any combination thereof.

In an aspect, a disclosed increase can comprise a 10%, 20%, 30%, 40%, 50%, 60%, 70%, 80%, 90%, 100%, or any amount of an increase when compared to a control subject (such as, for example, a subject that has not received HDAC11-activated M2 macrophages or a composition comprising HDAC11-activated M2 macrophages). In an aspect, a disclosed increase can comprise a 10-20%, 20-30%, 30-40%, 40-50%, 50-60%, 60-70%, 70-80%, 80-90%, or 90-100% or any amount of an increase when compared to a control subject (such as a subject that has not received HDAC11-activated M2 macrophages or a composition comprising HDAC11-activated M2 macrophages).

In an aspect, a disclosed decrease reduction can comprise a 10%, 20%, 30%, 40%, 50%, 60%, 70%, 80%, 90%, 100%, or any amount of a decrease when compared to a control subject (such as, for example, a subject that has not received HDAC11-activated M2 macrophages or a composition comprising HDAC11-activated M2 macrophages). In an aspect, a disclosed decrease can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated M2 macrophages or a composition comprising HDAC11-activated M2 macrophages).

In an aspect of a disclosed method of providing adaptive cell therapy to a subject, polarized M2 macrophages can be cryopreserved. In an aspect, cryopreserved polarized M2 macrophages can be thawed. In an aspect, thawing cryopreserved polarized M2 macrophages can be done by any technique known to the skilled person in the art. In an aspect, thawed polarized M2 macrophages can be administered to a subject. In an aspect of a disclosed method of providing adaptive cell therapy to a subject, resting or M0 macrophages can be cryopreserved prior to polarization and/or activation. In an aspect, cryopreserved resting or M0 macrophages can be thawed and then can be polarized and/or activated. In an aspect, thawing cryopreserved resting or M0 macrophages can be done by any technique known to the skilled person in the art. In an aspect, thawed resting or M0 macrophages can be polarized and/or activated and can then be administered to a subject.

In an aspect, a disclosed method of providing adaptive cell therapy to a subject can further comprise administering to the subject the HDAC11-activated M2 macrophages or a composition comprising HDAC11-activated M2 macrophages. In an aspect, administering can comprise intravenous administration of HDAC11-activated M2 macrophages or a composition comprising HDAC11-activated M2 macrophages. In an aspect, administering can comprise systemic administration or local administration of HDAC11-activated M2 macrophages or a composition comprising HDAC11-activated M2 macrophages.

In an aspect, local administration can comprise delivery to one or more of the subject's body systems having inflammation. In an aspect, the subject's one or more body systems having inflammation can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect, local delivery or local administration can refer to delivering or administering HDAC11-activated M2 macrophages or a composition comprising HDAC11-activated M2 macrophages directly to a target site within a subject. For example, HDAC11-activated M2 macrophages or a composition comprising HDAC11-activated M2 macrophages can be locally delivered by direct injection into an affected body system (such as one, for example, having inflammation). Local delivery or local administration can also include topical applications or localized injection techniques such as intramuscular, subcutaneous, and/or intradermal injection of HDAC11-activated M2 macrophages or a composition comprising HDAC11-activated M2 macrophages. In an aspect, local administration of HDAC11-activated M2 macrophages or a composition comprising HDAC11-activated M2 macrophages can comprise delivery to one or more body systems in need thereof. In an aspect, local administration or local delivery HDAC11-activated M2 macrophages or a composition comprising HDAC11-activated M2 macrophages can comprise a transdermal administration, administration via implant, administration via suppository, implantation via a biodegradable polymer or matrix, implantation via a biodegradable hydrogel, or any combination thereof.

In an aspect of a disclosed method of providing adaptive cell therapy to a subject, administering HDAC11-activated M2 macrophages or a composition comprising HDAC11-activated M2 macrophages can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, HDAC11-activated M2 macrophages or a composition comprising HDAC11-activated M2 macrophages can be administered via multiple routes of administration.

In an aspect of a disclosed method of providing adaptive cell therapy to a subject, following administration of HDAC11-activated M2 macrophages or a composition comprising HDAC11-activated M2 macrophages to the subject, inflammation in one or more of the subject's body systems can be diminished and/or decreased. In an aspect of a disclosed method of providing adaptive cell therapy to a subject, the decrease in inflammation in one or more of the subject's body systems can be measured objectively and/or subjectively. In an aspect of a disclosed method of providing adaptive cell therapy to a subject, following administration of HDAC11-activated M2 macrophages or a composition comprising HDAC11-activated M2 macrophages to the subject, the pathology of the subject's disease or disorder can be diminished and/or decreased. In an aspect of a disclosed method of providing adaptive cell therapy to a subject, wherein, following administration of HDAC11-activated M2 macrophages or a composition comprising HDAC11-activated M2 macrophages to the subject, the subject's symptoms can be diminished and/or decreased. In an aspect of a disclosed method of providing adaptive cell therapy to a subject, wherein, following administration of HDAC11-activated M2 macrophages or a composition comprising HDAC11-activated M2 macrophages to the subject, the subject's quality of life can be improved and/or enhanced. In an aspect, quality of life can be measured objectivity and/or subjectively. In an aspect of a disclosed method of providing adaptive cell therapy to a subject, wherein, following administration of HDAC11-activated M2 macrophages or a composition comprising HDAC11-activated M2 macrophages to the subject, the one or more transplanted organs can be not rejected. In an aspect of a disclosed method of providing adaptive cell therapy to a subject, wherein, following administration of HDAC11-activated M2 macrophages or a composition comprising HDAC11-activated M2 macrophages to the subject, the one or more transplanted organs can be spared from rejection.

In an aspect of a disclosed method of providing adaptive cell therapy to a subject, wherein, following administration of HDAC11-activated M2 macrophages or a composition comprising HDAC11-activated M2 macrophages to the subject, the healing of the subject's one or more chronic wounds can be improved and/or enhanced. In an aspect of a disclosed method of providing adaptive cell therapy to a subject, wherein improving and/or enhancing healing of one or more chronic wounds can comprise improving angiogenesis, innervation, cellular migration, or any combination thereof.

In an aspect of a disclosed method of providing adaptive cell therapy to a subject, wherein following administration of HDAC11-activated M2 macrophages or a composition comprising HDAC11-activated M2 macrophages to the subject, one or more of the subject's body systems can experience and/or show signs of cellular regeneration and/or cellular repair. In an aspect of a disclosed method of providing adaptive cell therapy to a subject, wherein the one or more body systems experiencing and/or showing signs of regeneration and/repair can comprise the cardiovascular system. In an aspect of a disclosed method of providing adaptive cell therapy to a subject, wherein previously ischemic and/or infarcted and/or languishing areas of the cardiovascular system can show signs of regeneration and/or repair. In an aspect of a disclosed method of providing adaptive cell therapy to a subject, the one or more body systems experiencing and/or showing signs of regeneration and/repair can comprise the central nervous system and/or peripheral nervous system.

In an aspect of a disclosed method of providing adaptive cell therapy to a subject, wherein previously ischemic and/or infarcted/or languishing areas of the central nervous system and/or peripheral nervous system can show signs of regeneration and/or repair. In an aspect of a disclosed method of providing adaptive cell therapy to a subject, wherein the one or more body systems experiencing and/or showing signs of regeneration and/repair can comprise the integumentary system.

In an aspect of a disclosed method of providing adaptive cell therapy to a subject, wherein the one or more body systems experiencing and/or showing signs of regeneration and/repair can comprise the digestive system. In an aspect, a disclosed method of providing adaptive cell therapy to a subject can further comprise diminishing and/or decreasing inflammation in one or more of the subject's body systems. Body systems are disclosed supra. In an aspect, a decrease in inflammation in one or more of the subject's body systems can be measured objectively and/or subjectively. In an aspect, a disclosed method of providing adaptive cell therapy to a subject can further comprise diminishing and/or decreasing the pathology of the subject's disease or disorder. In an aspect, a disclosed method of providing adaptive cell therapy to a subject can further comprise diminishing and/or decreasing the subject's symptoms.

In an aspect, a disclosed method of providing adaptive cell therapy to a subject can further comprise improving and/or enhancing the subject's quality of life. In an aspect, quality of life can be measured objectivity and/or subjectively. In an aspect, a disclosed method of providing adaptive cell therapy to a subject can further comprise preventing the rejection of one or more transplanted organs in the subject. In an aspect, a disclosed method of providing adaptive cell therapy to a subject can further comprise minimizing the risk of rejection of one or more transplanted organs in the subject.

In an aspect, a disclosed method of providing adaptive cell therapy to a subject can further comprise and/or enhancing the healing of one or more chronic wounds in the subject. In an aspect, a disclosed method of providing adaptive cell therapy to a subject can further comprise and/or enhancing healing of one or more chronic wounds. In an aspect, a disclosed method of providing adaptive cell therapy to a subject can further comprise improving and/or enhancing angiogenesis, innervation, cellular migration, or any combination thereof in and/or around one or more chronic wounds in the subject. In an aspect, a disclosed method of providing adaptive cell therapy to a subject can further comprise improving and/or enhancing cellular regeneration and/or cellular repair or one or more of the subject's affected body systems. In an aspect, a disclosed method of providing adaptive cell therapy to a subject can further comprise improving and/or enhancing cellular regeneration and/or cellular repair in the subject's central nervous system and/or peripheral nervous system. In an aspect, a disclosed method of providing adaptive cell therapy to a subject can further comprise improving and/or enhancing cellular regeneration and/or cellular repair in the subject's integumentary system. In an aspect, a disclosed method of providing adaptive cell therapy to a subject can further comprise improving and/or enhancing cellular regeneration and/or cellular repair in the subject's digestive system.

In an aspect, a disclosed method of providing adaptive cell therapy to a subject can further comprise repeating one or more times one or more steps of a disclosed method. In an aspect of a disclosed method of providing adaptive cell therapy to a subject a disclosed obtaining step can be repeated, a disclosed isolating step can be repeated, a disclosed contacting step can be repeated, a disclosed activating step can be repeated, a disclosed polarizing step can be repeated, a disclosed incubating step can be repeated, a disclosed administering step can be repeated, or any combination thereof. In an aspect, a disclosed method of providing adaptive cell therapy to a subject can further comprise repeating an obtaining step, repeating an isolating step, repeating a contacting step, repeating an activating step, repeating a polarizing step, repeating an incubating step, repeating an administering step, or repeating one or more time any combination of steps thereof.

In an aspect of a disclosed method of providing adaptive cell therapy to a subject, a disclosed improvement and/or enhancement (such as, for example, in the subject's quality of life and/or movement and/or would healing and/or angiogenesis, etc.) can comprise a 10%, 20%, 30%, 40%, 50%, 60%, 70%, 80%, 90%, 100%, or any amount of improvement and/or enhancement when compared to a control subject (such as, for example, a subject that has not received HDAC11-activated M2 macrophages or a composition comprising HDAC11-activated M2 macrophages). In an aspect, a disclosed improvement and/or enhancement (such as, for example, in the subject's quality of life and/or movement and/or would healing and/or angiogenesis, etc.) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not HDAC11-activated M2 macrophages or a composition comprising HDAC11-activated M2 macrophages).

In an aspect of a disclosed method of providing adaptive cell therapy to a subject, a disclosed diminishment (such as, for example, in the subject's pain and/or wound size and/or restrictions) can comprise a 10%, 20%, 30%, 40%, 50%, 60%, 70%, 80%, 90%, 100%, or any amount of a decrease when compared to a control subject (such as, for example, a subject that has not received HDAC11-activated M2 macrophages or a composition comprising HDAC11-activated M2 macrophages). In an aspect, a disclosed diminishment (such as, for example, in the subject's pain and/or wound size and/or restrictions) can comprise a 10-20%, 20-30%, 30-40%, 40-50%, 50-60%, 60-70%, 70-80%, 80-90%, or 90-100% or any amount of a decrease when compared to a control subject (such as a subject that has not received HDAC11-activated M2 macrophages or a composition comprising HDAC11-activated M2 macrophages).

In an aspect, a disclosed method of providing adaptive cell therapy to a subject can further comprise administering to a disclosed subject one or more therapeutic agents and/or active agents. In an aspect, one or more disclosed therapeutic agents and/or active agents can comprise an immune modulator. In an aspect, a disclosed immune modulator can refer to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions.

In an aspect of a disclosed method of providing adaptive cell therapy to a subject, administering can comprise intravenous administration of one or more therapeutic agents and/or active agents. In an aspect, administering can comprise systemic administration or local administration of one or more therapeutic agents and/or active agents.

In an aspect of a disclosed method of providing adaptive cell therapy to a subject, local delivery or local administration can refer to delivering or administering one or more therapeutic agents and/or active agents directly to a target site within a subject. For example, the one or more therapeutic agents and/or active agents can be locally delivered by direct injection into an affected body system (such as one, for example, having inflammation). Local delivery or local administration can also include topical applications or localized injection techniques such as intramuscular, subcutaneous, and/or intradermal injection of one or more therapeutic agents and/or active agents. In an aspect, local administration of one or more therapeutic agents and/or active agents can comprise delivery to one or more body systems in need thereof. In an aspect, local administration or local delivery of one or more therapeutic agents and/or active agents can comprise a transdermal administration, administration via implant, administration via suppository, implantation via a biodegradable polymer or matrix, implantation via a biodegradable hydrogel, or any combination thereof. A skilled clinician can determine the best route of administration for a subject at a given time.

In an aspect of a disclosed method of providing adaptive cell therapy to a subject, HDAC11-activated M2 macrophages or a composition comprising HDAC11-activated M2 macrophages can be administered to the subject prior to, concurrent with, or after administration of one or more therapeutic agents and/or active agents. In an aspect, HDAC11-activated M2 macrophages or a composition comprising HDAC11-activated M2 macrophages can be administered one or more times and the one or more therapeutic agents and/or active agents can be administered one or more times. In an aspect of a disclosed method of providing adaptive cell therapy to a subject, HDAC11-activated M2 macrophages or a composition comprising HDAC11-activated M2 macrophages can be administered one time and the one or more therapeutic agents and/or active agents can be administered one or more times. Dosing schedules can be determined by skilled person in the art.

In an aspect of a disclosed method of providing adaptive cell therapy to a subject, the subject's physiology and/or functionality can be restored to normal or near normal levels (e.g., with little or no inflammation or little or no signs and/or symptoms of inflammation). In an aspect of a disclosed method of providing adaptive cell therapy to a subject, the disclosed subject's behavior and/or physiology can be modulated.

In an aspect, a therapeutically effective amount of HDAC11-activated M2 macrophages or a composition comprising HDAC11-activated M2 macrophages can comprise a range of macrophages activated by from about 50 nM to about 25 M of an HDAC11-inhibitor. In an aspect, a therapeutically effective amount of HDAC11-activated M2 macrophages or a composition comprising HDAC11-activated M2 macrophages can comprise a range of macrophages that elicit and/or provoke one or more measurable anti-inflammatory responses.

In an aspect, a disclosed method of providing adaptive cell therapy to a subject can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinician can decide to change an aspect of the subject's treatment, and/or change the subject's diagnosis or prognosis.

In an aspect, a disclosed method of providing adaptive cell therapy to a subject can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect of a disclosed method of providing adaptive cell therapy to a subject, modifying the treating step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof.

In an aspect of a disclosed method of providing adaptive cell therapy to a subject, modifying the administering step can comprise changing the amount of HDAC11-activated M2 macrophages or a composition comprising HDAC11-activated M2 macrophages administered to the subject, changing the frequency of administration of HDAC11-activated M2 macrophages or a composition comprising HDAC11-activated M2 macrophages, changing the duration of administration of HDAC11-activated M2 macrophages or a composition comprising HDAC11-activated M2 macrophages, changing the route of administration of HDAC11-activated M2 macrophages or a composition comprising HDAC11-activated M2 macrophages, or any combination thereof.

In an aspect, a disclosed method of providing adaptive cell therapy to a subject can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method of providing adaptive cell therapy to a subject, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer HDAC11-activated M2 macrophages or a composition comprising HDAC11-activated M2 macrophages. In an aspect, metabolic and/or physiologic data can inform the clinician when make subsequent treatment decisions.

Disclosed herein is a method of providing adaptive cell therapy, the method comprising isolating M0 or naïve macrophages from a subject in need thereof, contacting the M0 or naïve macrophages ex vivo with a one or more HDAC11 inhibitors to produce HDAC11-activated macrophage, polarizing the HDAC11-activated macrophages to an anti-inflammatory phenotype, and administering to the subject the HDAC11-activated and polarized macrophages.

Disclosed herein is a method of providing adaptive cell therapy, the method comprising isolating M0 or naïve macrophages from a subject in need thereof, polarizing the M0 or naïve macrophages to an anti-inflammatory phenotype ex vivo, contacting the polarized macrophages with a one or more HDAC11 inhibitors to produce HDAC11-activated macrophage, and administering to the subject the HDAC11-activated and polarized macrophages.

### K. Miscellaneous

Disclosed herein is adoptive cell therapy with M2 macrophages treated ex-vivo with HDAC11 inhibitors to enhance chronic wound healing. Disclosed herein is adoptive cell therapy with M2 macrophages treated ex-vivo with HDAC11 inhibitors to enhance healing such as foot ulcers and other chronic wounds in diabetic patients. Disclosed herein is topical application of HDAC11 inhibitors to chronic wounds to enhance wound healing and subside skin inflammatory diseases. Disclosed herein is adoptive cell therapy with M2 macrophages treated ex-vivo with HDAC11 inhibitors to prevent transplant rejection in skin and other organs. Disclosed herein is adoptive cell therapy with M2 macrophages treated ex-vivo with HDAC11 inhibitors to facilitate heart regeneration and repair after ischemic injury. Disclosed herein is treating inflammatory diseases such as Systemic Lupus Erythematosus (SLE), irritable bowel disease (IBD) with HDAC11 treated M2 macrophages. Disclosed herein is adoptive cell therapy with M2 macrophages treated ex-vivo with HDAC11 inhibitors to treat burn injuries to enhance repair. Disclosed herein are autoimmune disorders associated with uncontrolled inflammation. Disclosed herein is systemic treatment with HDAC11 inhibitor as API for all the above-mentioned inflammation associate applications which can affect macrophage function towards anti-inflammatory phenotype.

### EXAMPLES

The Examples that follow are illustrative of specific aspects of the invention, and various uses thereof. They set forth for explanatory purposes only and are not to be taken as limiting the invention.

### Example 1

### HDAC11 Regulated IL10 Gene Expression in Macrophages

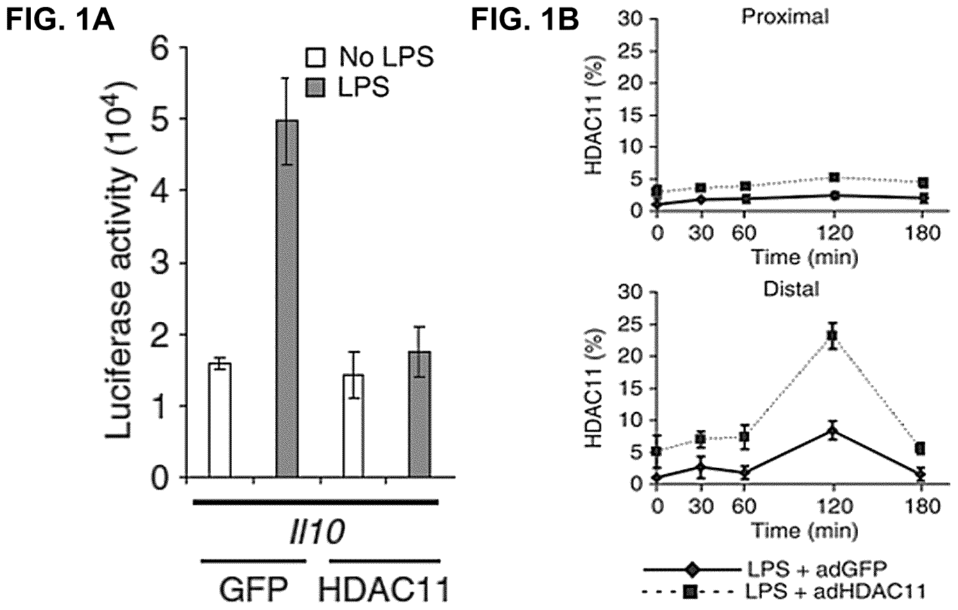

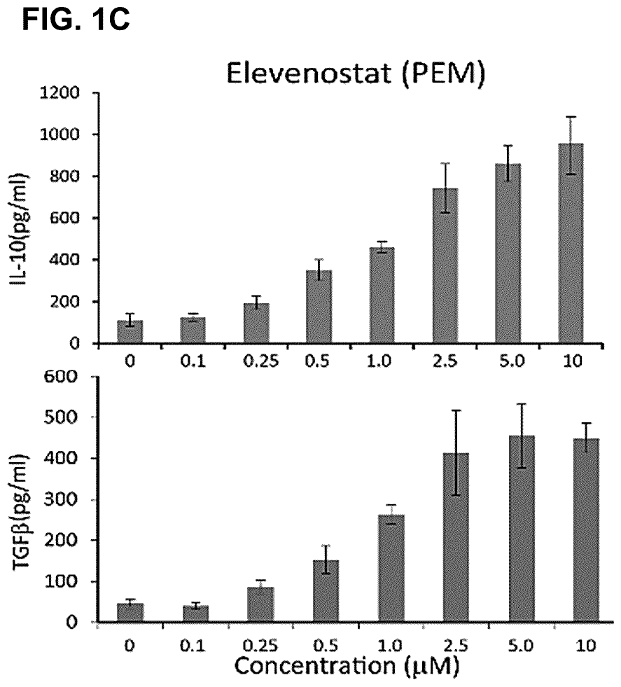

The work described herein demonstrate-d the immunosuppressive function of HDAC11 inhibition in macrophages. Upon treatment with M-CSF, bone marrow-derived monocytes (BMDM) differentiate into naïve (M0) macrophages. In the case of peritoneal exudate macrophages (PEMs), naïve M0 macrophages are isolated from the peritoneal cavity. These M0 macrophages are pre-treated with HDAC11 inhibitor followed by polarization to M2 macrophages with IL4 and IL13 cytokines or other cytokine mix including but not limited to IL10 and TGF beta. Here, co-transfection of RAW264.7 murine macrophages with plasmids expressing cither GFP (control) or HDAC11 and luciferase reporter plasmid driven by the distal IL10 gene promoter indicated that HDAC11 suppressed IL10 reporter plasmid as evidenced by the luciferase activity (FIG. 1A). A chromatin immunoprecipitation assay further confirmed that HDAC11 binded preferentially to the distal promoter region of IL10, and negatively regulated its gene expression (FIG. 1B). Inhibition or genetic knockdown of HDAC11 relieved the repression and resulted in IL10 gene expression (Villagra A, et al. (2009) Nat Immunol. 10:92-100). Therefore, peritoneal exudated macrophages (PEMs) were treated with the HDAC11 specific inhibitor, elevenostat, which increased the expression of IL-10 and TGFβ cytokines in a dose-dependent manner (FIG. 1C). This confirmed that the HDAC11 inhibitor was highly specific, and that it could recapitulate the effect of HDAC11 genetic knock down.

### Example 2

### HDAC11 Inhibition Augmented M2 Phenotype

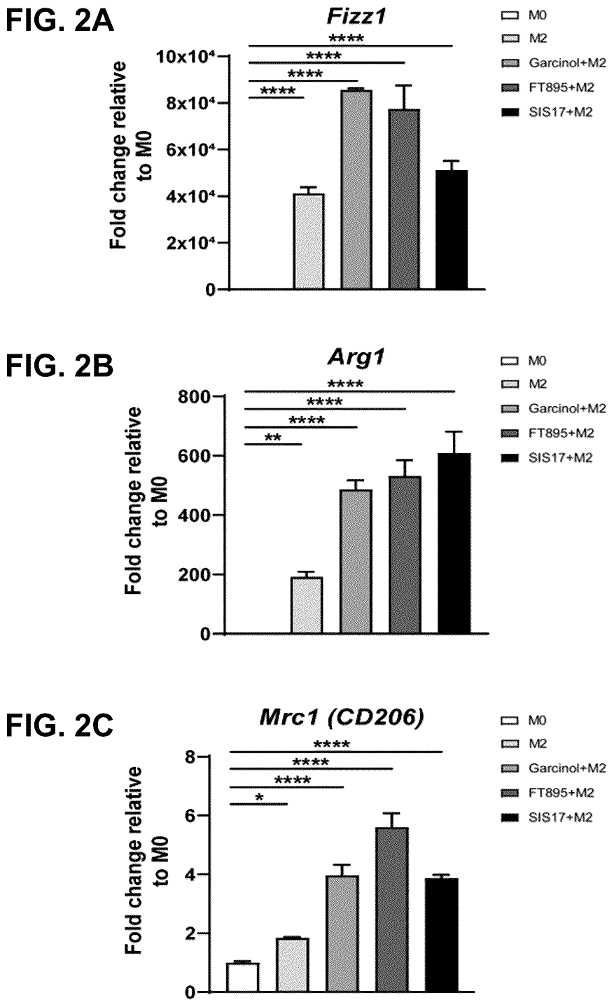

Next, HDAC11 inhibitors were evaluated for their ability to modulate the function of macrophages. SIS17 is known to inhibit the demyristoylation activity of HDAC11 without inhibiting other HDACs (Son S I, et al. (2019) ACS Chemical Biology. 14 (7): 1393-1397). Another HDAC11 inhibitor, FT895 is a potent and selective HDAC11 inhibitor with excellent intracellular activity and pharmacokinetic profile (Martin M W, et al. (2018) Bioorg Med Chem Lett. 28 (12): 2143-2147). Garcinol, a natural product is another known HDACIII with anti-oxidant and anti-inflammatory properties (Son S I, et al. (2020) ACS Chemical Biology. 15 (11): 2866-2871). These commercially available HDAC11 inhibitors were tested on macrophages in-vitro to identify their macrophage modulatory properties. Next, BMDMs were isolated and polarized towards M2 phenotype with HDAC11 inhibitor pre-treatment overnight. Investigation into the expression of fibrosis marker Fizz/revealed that polarization of naïve (M0) macrophages towards M2 phenotype significantly increased Fizz/expression. Pre-treatment with the HDAC11 inhibitors Garcinol, SIS17, and FT895 further enhanced the expression of Fizz1 (FIG. 2B), indicating that HDAC11 inhibition in macrophages strongly favored M2 phenotype. Finally, analysis of Arg1 and Mrc1 M2 macrophage markers also increased the expression of Arg1 and Mrc1 after pre-treatment with SIS17 or did not affect in the case of FT895 (FIG. 2B and FIG. 2C). This data shows the role of HDAC11 in regulating M2 phenotype of macrophages. Thus far, these data indicate that pharmacological inhibition of HDAC11 or genetic knockout of HDAC11 favored an anti-inflammatory and wound healing M2 phenotype.

### Summary of Experiments

As described herein, exposure to bacterial lipopolysaccharide (LPS) induces an inflammatory response in macrophages. This inflammatory response elicited an anti-inflammatory response, which was evident by the activation of IL10 promoter driven luciferase reporter expression as shown in FIG. 1A. However, treatment with an HDAC11 inhibitor significantly suppressed the IL10 reporter activity, indicating that HDAC11 was involved in the suppression of IL10 gene. This was further validated with chromatin immunoprecipitation assays in FIG. 1B in which HDAC11 predominantly bound to the distal promoter region of IL10 gene.

At a protein level, the inhibition of HDAC11 with elevenostat was demonstrated in PEMs, and there was a dose dependent increase in the anti-inflammatory cytokine IL-10 and Tgf-β as determined by ELISA assay. Furthermore, treatment of bone marrow derived macrophages with different HDAC11 inhibitors consistently increased the expression of M2 markers such as Fizz1, Arg1, and Mrc1 in M2 polarized and anti-inflammatory phenotype. These data comprehensively demonstrate that HDAC11 plays a significant role in controlling anti-inflammatory responses in macrophages. Therefore, HDAC11 inhibitor treated M2 macrophages as a cell therapy and or topical HDAC11 inhibitor application are excellent to treat inflammatory diseases.

The disclosed approach to use HDAC11 inhibitors to treat inflammation associated diseases ranging from chronic wounds nonhealing wounds to autoinflammatory diseases is unique and novel because class and isoform specific histone deacetylase inhibitors have less toxicity and high selectivity to HDAC11 inhibition compared to the pan-HDAC inhibitors. The experiments discussed herein demonstrated that isolated macrophages can now be reprogrammed outside the body (ex-vivo) and that these isolated macrophages can be polarized towards anti-inflammatory wound healing M2 phenotype. Thus, administration of these M2 macrophages provides an anti-inflammatory effect that can be applied to most inflammation-associated conditions. Because macrophages are treated ex-vivo with HDAC11 inhibitors, and HDAC11 treated M2 macrophages are used as biological agents to treat inflammatory disease conditions, concerns associated with toxicity are thereby advantageously eliminated. Also, macrophages are derived from one's own monocytes; therefore, concerns regarding graft-vs-host rejection reactions are also advantageously eliminated. Moreover, as the M2 phenotype is the default state of predominant tissue resident macrophages, cell therapy with HDAC11 treated macrophages is like likely to elicit adverse effects. Finally, the flexibility in the methods discussed herein affords lateral expansion of monocytes ex-vivo to generate macrophages for cell therapy.

1. A method of treating a subject, the method comprising:
administering to a subject in need thereof a therapeutically effective amount of HDAC11-activated M2 macrophages or a composition comprising HDAC11-activated M2 macrophages, wherein following the administering step, inflammation in one or more of the subject's body systems is decreased and/or diminished.

2. The method of claim 1, further comprising generating HDAC11-activated M2 polarized macrophages.

3. The method of claim 2, wherein generating HDAC11-activated macrophages can be an ex vivo process.

4. The method of claim 2, wherein generating HDAC11-activated macrophages comprises contacting M0 macrophages or naïve macrophages with one or more HDAC11 inhibitors.

5. The method of claim 4, further comprising polarizing the HDAC11-activated macrophages into a M2 or anti-inflammatory phenotype.

6. The method of claim 5, wherein polarizing the HDAC11-activated macrophages into a M2 or anti-inflammatory phenotype comprise contacting the HDAC11-activated macrophages with IL-4, IL-10, IL-13, TGFβ-1, PGE2, or any combination thereof.

7. The method of claim 1, further comprising administering to the subject a therapeutically effective amount of one or more HDAC11 inhibitors or one or more compositions comprising a therapeutically effective amount of one or more HDAC11 inhibitors.

8. The method of claim 7, wherein the therapeutically effective amount of an HDAC11 inhibitor comprises from about 50 nM to about 25 μM.

9. The method of claim 1, wherein the subject has one or more chronic wounds.

10. The method of claim 9, wherein the one or more chronic wounds comprise infected and/or traumatic wounds, arterial ulcers, venous ulcers, pressure ulcers, diabetic ulcers, ischemic ulcers, or any combination thereof.

11. (canceled)

12. The method of claim 1, wherein the subject has one or more inflammatory diseases.

13. The method of claim 1, wherein the subject has incurred an ischemic injury.

14. The method of claim 13, wherein the ischemic injury affects the subject's brain and/or the subject's heart.

15. (canceled)

16. The method of claim 1, wherein the subject has an autoinflammatory disease (AIF) and/or an autoimmune disease (AD).

17. The method of claim 4, wherein the one or more HDAC11 inhibitors comprise elevenostat (JB3-22), garcinol, SIS7, SIS17, FT895, romidepsin, quisinostat, or any combination thereof.

18. The method of claim 1, wherein administering comprises systemic administration and/or local administration to one or more of the subject's body systems having inflammation.

19. (canceled)

20. The method of claim 1, wherein the one or more body systems having inflammation comprise the cardiovascular system, the digestive system, the endocrine system, the lymphatic system, the muscular system, the nervous system, the reproductive system, the respiratory system, the skeletal system, the urinary system, the integumentary system, or any combination thereof.

21. The method of claim 1, wherein following the administering step, the subject's symptoms can be diminished and/or decreased, the subject's quality of life can be improved and/or enhanced, one or more of the subject's body systems can experience and/or show signs of cellular regeneration and/or cellular repair, or any combination thereof.

22. (canceled)

23. (canceled)

24. (canceled)

25. (canceled)

26. (canceled)

27. (canceled)

28. (canceled)

29. (canceled)

30. The method of claim 7, wherein the one or more HDAC11 inhibitors comprise elevenostat (JB3-22), garcinol, SIS7, SIS17, FT895, romidepsin, quisinostat, or any combination thereof.

31. The method of claim 7, wherein following the administering step, the subject's symptoms can be diminished and/or decreased, the subject's quality of life can be improved and/or enhanced, one or more of the subject's body systems can experience and/or show signs of cellular regeneration and/or cellular repair, or any combination thereof.
